SEQUENCE LISTING (9 sequences)

SEQ ID NO: 1 - DNA, 20 bp, Unknown
  sgRNA
acatttcaattatatgttaa

SEQ ID NO: 2 - DNA, 20 bp, Unknown
  sgRNA
atactagttaagcttttcct

SEQ ID NO: 3 - DNA, 20 bp, Unknown
  sgRNA
aaaccagcattgccacctgg

SEQ ID NO: 4 - DNA, 20 bp, Unknown
  sgRNA
ccaggtggcaatgctggttt

SEQ ID NO: 5 - DNA, 160 bp, Unknown
  repair template
agccccatagttggtgctcaataaatgctagtaatatttacttgtggcttactggttcctcaagattccttaaaatctgatggcatcagaagagactatctcactgttatcatgacatggacatcccgtgcatgcctgtatttgaacacttgtctcattg

SEQ ID NO: 6 - DNA, 19 bp, Unknown
  forward primer
  misc_feature (10)..(10): N = biotinylated bridge linker
cgcgatatcntatctgact

SEQ ID NO: 7 - DNA, 19 bp, Unknown
  reverse primer
gtcagataagatatcgcgt

SEQ ID NO: 8 - DNA, 20 bp, Unknown
  forward primer
acgcgatatcttatctgact

SEQ ID NO: 9 - DNA, 20 bp, Unknown
  reverse primer
agtcagataagatatcgcgt

(19) United States
(12) Patent Application Publication
(10) Pub. No.: US 2019/0005191 A1
(43) Pub. Date: Jan. 3, 2019

(54) CHROMOSOME NEIGHBORHOOD STRUCTURES AND METHODS RELATING THERETO

(71) Applicant: WHITEHEAD INSTITUTE FOR BIOMEDICAL RESEARCH, Cambridge, MA (US)

(72) Inventors: Denes Hnisz, Cambridge, MA (US); Richard A. Young, Boston, MA (US); Diego R. Borges-Rivera, Belmont, MA (US); Abraham S. Weintraub, Somerville, MA (US); Xiong Ji, Cambridge, MA (US); Daniel B. Dadon, Cambridge, MA (US); Zi Peng Fan, Waltham, MA (US); Tong Ihn Lee, Somerville, MA (US)

(21) Appl. No.: 15/744,685

(22) PCT Filed: Jul. 14, 2016
(86) PCT No.: PCT/US16/42367

§ 371 Date: Jan. 12, 2018

Related U.S. Application Data
(60) Provisional application No. 62/192,561, filed on Jul. 14, 2015.
(60) Provisional application No. 62/192,559, filed on Jul. 14, 2015.
(60) Provisional application No. 62/252,393, filed on Nov. 6, 2015.

(51) Int. Cl. G06F 19/20; C12Q 1/6886

(52) U.S. Cl. CPC G06F 19/20; C12Q 1/6886

(57) ABSTRACT

Work described herein reveals 3D regulatory landscapes of hESCs representative of early human development. This work also demonstrates that cohesin-associated CTCF loops, and the cohesin-associate enhancer-promoter loops within them, dominate the organization of TADs. The CTCF-CTCF loops form a chromosomal scaffold of insulated neighborhoods that are largely preserved in vertebrates, and enhancer-promoter interactions occur within these neighborhoods. Genes are regulated in the context of conserved insulated neighborhood structures. Loss of neighborhood structures occurs frequently in cancer cells, and proto-oncogenes can be activated by genetic alterations that disrupt specific 3D chromosome structures.

### RELATED APPLICATIONS

This application claims the benefit of U.S. Provisional Application No. 62/192,559 and U.S. Provisional Application No. 62/192,561, both filed on Jul. 14, 2015, and U.S. Provisional Application No. 62/252,393, filed on Nov. 6, 2015. The entire teachings of the above applications are incorporated herein by reference.

### GOVERNMENT SUPPORT

This invention was made with government support under Grant Nos. HG002668 and CA109901 awarded by the National Institutes of Health. The government has certain rights in the invention.

### BACKGROUND OF THE INVENTION

The mammalian genome is organized in a 3D topology that is thought to contribute to the regulation of gene expression, in part by creating constraints that produce regions of active and repressed transcription. Regulatory elements and genes are thought to be physically and functionally connected within conserved chromosome structures called Topologically Associating Domains (TADs), but the mechanisms that generate and maintain this 3D regulatory landscape are not yet understood.

Tumor cell gene expression programs are typically driven by somatic mutations that alter the coding sequence or cause overexpression of proto-oncogenes (D. Stehelin et al., Nature, 11 Mar. 1976, 260:170), and identifying such mutations in patient genomes is a major goal of cancer genomics. Somatic mutations that cause dysregulation of proto-oncogenes frequently involve alterations that bring transcriptional enhancers into proximity with these genes. In normal cells, transcriptional enhancers interact with their target genes through the formation of DNA loops (D. Carter, et al., Nat Genet, December 2002, 32:623). Two types of chromosome structures have been implicated in constraining the regulatory activity of enhancers to specific genes: (TADs) and insulated neighborhoods. TADs are megabase-sized chromosome domains, and insulated neighborhoods are DNA loops within TADs that are formed by interactions between two DNA sites bound by the chromosome structure regulators CTCF and cohesin (J. M. Dowen, Cell, 9 Oct. 2014, 159:374). A better understanding of the 3D regulatory landscape, including identification of enhancers, insulators and cohcsin-associated chromatin interactions, the role of insulated neighborhoods, and the impact of disruption of neighborhoods will facilitate the diagnosis and treatment of a wide array of diseases.

### SUMMARY OF THE INVENTION

Work described herein reveals 3D regulatory landscapes of hESCs representative of early human development. This work also demonstrates that cohesin-associated CTCF loops, and the cohesin-associate enhancer-promoter loops within them, dominate the organization of TADs. The CTCF-CTCF loops form a chromosomal scaffold of insulated neighborhoods that are largely preserved in vertebrates, and enhancer-promoter interactions occur within these neighborhoods. Genes are regulated in the context of conserved insulated neighborhood structures. Loss of neighborhood structures occurs frequently in cancer cells, and proto-oncogenes can be activated by genetic alterations that disrupt specific 3D chromosome structures.

In some aspects, the invention provides a method of identifying one or more differences in a regulatory pathway between two cells comprising obtaining expression data for at least one enhancer from each cell from an insulated neighborhood conserved between the two cells and comparing said expression data to identify differential activity of said enhancer on at least one target gene.

In some embodiments, the cells are embryonic stem cells. In some embodiments, the cells are iPS cells, and in further embodiments, one cell is naïve and one cell is primed. In some embodiments, one cell is a more differentiated cell type than the other cell.

In some aspects, the invention provides a method for identifying a Topologically Associating Domain (TAD) comprising identifying TAD boundaries utilizing ChIA-PET data and identifying a TAD between two TAD boundaries.

In some embodiments, the ChIA-PET data is cohesin ChIA-PET data. In some embodiments the ChIA-PET data is processed using a Hidden Markov algorithm.

In some aspects, the invention provides a method of inhibiting activation of a proto-oncogene by an enhancer, wherein one of the proto-oncogene or enhancer is located within an insulated neighborhood, comprising stabilizing the boundary of said insulated neighborhood such that disruption of the neighborhood is reduced, thereby inhibiting interaction of the enhancer with the proto-oncogene.

In some embodiments, the proto-oncogene is located within an insulated neighborhood. In some embodiments, the enhancer is located within an insulated neighborhood. In some embodiments, the enhancer and the proto-oncogene are each located within an insulated neighborhood, and wherein said insulated neighborhoods are different from one another.

In some aspects, the invention provides a method of identifying a super-enhancer in a 3D regulatory landscape of a cell comprising examining all enhancer activity within an insulated neighborhood, and stitching all enhancers located within the insulated neighborhood together to form a super-enhancer.

In some embodiments, the super-enhancer is identified by performing chromatin immunoprecipitation high throughput sequencing (ChTP-Seq). In some embodiments, the enhancers are located within a predetermined distance of each other (e.g., within 12.5 kb of each other). In some embodiments, the method further comprises identifying a gene associated with the super-enhancer. In some embodiments, the associated gene is identified by proximity to the super-enhancer. In some embodiments, the associated gene is a proto-oncogene. In some embodiments, the associated gene is located within an insulated neighborhood different from the insulated neighborhood in which the super-enhancer is located.

In some aspects, the invention provides a method of identifying a super-enhancer in a 3D regulatory landscape of a cell comprising identifying genomic regions of DNA within the cell enriched for H3K27ac signal, stitching the enriched regions together if within 12.5 kb of each other, ranking stitched regions by H3K27ac signal, and identifying a ranked stitched regions as a super-enhancer if the ranked stitched region falls above a threshold at which two classes of enhancers are separable.

In some aspects, the invention provides a method of identifying a disruption in an insulated neighborhood boundary comprising identifying a proto-oncogene of interest, identifying an insulated neighborhood within which the proto-oncogene is located, and examining the proto-oncogene neighborhood for disruptions in a proto-oncogene neighborhood boundary.

In some embodiments, an enhancer (e.g., a super-enhancer) is located outside the proto-oncogene neighborhood. In some embodiments, the enhancer is located within an insulated neighborhood that is different from the proto-oncogene neighborhood. In some embodiments, the method further comprises identifying activation of a proto-oncogene located within the proto-oncogene neighborhood by an enhancer located outside the proto-oncogene neighborhood. In some embodiments, the disruption in the proto-oncogene neighborhood boundary is a mutation or a microdeletion in a CTCF-CTCF loop anchor region. In some embodiments, the disruption is a deletion, and the proto-oncogene neighborhood boundary overlaps the deletion by at least one 1 bp.

In some aspects, the invention provides a method of identifying a disruption in an insulated neighborhood boundary comprising identifying at least one proto-oncogene of interest, identifying candidate neighborhoods comprised of CTCF-CTCF loops wherein the transcription start site of the at least one proto-oncogene is located within the neighborhood, examining the proto-oncogene neighborhoods for microdeletions or other mutations, and determining if any identical microdeletions overlap proto-oncogene neighborhood boundaries.

In some aspects, the invention provides a method of screening for cancer, comprising identifying a proto-oncogene of interest, wherein the proto-oncogene is located within an insulated neighborhood, examining the proto-oncogene insulated neighborhood for disruptions in a boundary of the proto-oncogene insulated neighborhood, and measuring expression of the proto-oncogene, wherein elevated levels of the proto-oncogene indicated a likelihood of cancer.

In some aspects, the invention provides a method of treating a cancer involving an activated proto-oncogene, comprising administering to a patient in need of such treatment an effective amount of an agent that repairs a deletion or other disruption in an insulated neighborhood boundary, wherein the activated proto-oncogene is located within the insulated neighborhood, thereby decreasing expression of the proto-oncogene such that the cancer is treated.

In some aspects, the invention provides a method of identifying an agent that stabilizes an insulated neighborhood, wherein the insulated neighborhood has a disrupted boundary, comprising transfecting a cell with a super-enhancer and the insulated neighborhood under conditions suitable for the super-enhancer to drive high levels of expression of a proto-oncogene that is associated with the super-enhancer and is located within the insulated neighborhood, contacting the cell with a test agent, and measuring the level of expression of the proto-oncogene, wherein decreased expression of the proto-oncogene in the presence of the test agent indicates that the test agent is an agent that stabilizes an insulated neighborhood.

In some embodiments, the agent disrupts the super-enhancer associated with the proto-oncogene. In some embodiments, the super-enhancer is located outside the insulated neighborhood. In some embodiments, the agent repairs a disruption in the disrupted insulated neighborhood boundary. In some embodiments, expression of the proto-oncogene is measured at least in part by measuring the level of a gene product encoded by the proto-oncogene or by measuring activity of a gene product encoded by the proto-oncogene. In some embodiments, the gene product is mRNA or polypeptide encoded by the gene.

In some aspects, the invention provides a method of identifying an agent that disrupts a super-enhancer associated with a proto-oncogene comprising transfecting a cell with a super-enhancer and an associated proto-oncogene under conditions suitable for the super-enhancer to drive high levels of expression of the proto-oncogene, wherein the proto-oncogene is located within an insulated neighborhood, contacting the cell with a test agent, and measuring the level of expression of the proto-oncogene, wherein decreased expression of the proto-oncogene in the presence of the test agent indicates that the test agent is an agent that disrupts the super-enhancer associated with the proto-oncogene.

In some aspects, the invention provides a method of identifying a screening agent that identifies a disruption in an insulated neighborhood boundary, comprising transfecting a cell with a super-enhancer and an associated proto-oncogene, wherein the proto-oncogene is located within an insulated neighborhood, contacting the cell with a screening agent, and measuring the level of expression of the screening agent, wherein increased expression of the screening agent indicates that the proto-oncogene is activated.

In some aspects, the invention provides a method of screening an individual for a pre-disposition of cancer comprising identifying a proto-oncogene located within an insulated neighborhood, and determining if a boundary of the insulated neighborhood includes a disruption, wherein a disruption in the insulated neighborhood boundary indicates an increased risk of cancer.

In some embodiments, the method further includes identifying if an enhancer is located within the vicinity of the insulated neighborhood. In some embodiments, the disruption in the insulated neighborhood boundary is a deletion in a CTCF loop binding site.

In some aspects, the invention provides a method of identifying a candidate target for treating a cancer, comprising detecting a disrupted boundary of an insulated neighborhood that contains one or more proto-oncogenes in genomic DNA derived from the cancer, and identifying an enhancer located outside of and in proximity to the insulated neighborhood, thereby identifying the proto-oncogene and the enhancer as candidate targets for treating the cancer.

In some embodiments, the enhancer is a super-enhancer. In some embodiments, the proto-oncogene is not expressed when the insulated neighborhood boundary is not disrupted. In some embodiments, the method further comprises measuring expression of the proto-oncogene in a sample comprising cancer cells derived from the cancer, wherein higher expression of the proto-oncogene in cancer cells as compared to normal cells indicates that the proto-oncogene or the enhancer is a target for treating the cancer. In some embodiments, the method further comprises identifying an agent that inhibits activity of the enhancer. In some embodiments, the method further comprises identifying an agent that inhibits expression of the proto-oncogene or inhibits activity of a gene product of the proto-oncogene. In some embodiments, the method further comprises contacting a cancer cell having a disruption in the insulated neighborhood boundary with an agent that inhibits activity of the enhancer, inhibits expression of the proto-oncogene, or inhibits activity of a gene product of the proto-oncogene. In some embodiments, the method further comprises administering to a subject in need of treatment for a cancer comprising cells that have a disruption in the insulated neighborhood boundary, an agent that inhibits activity of the enhancer, inhibits expression of the proto-oncogene, or inhibits activity of a gene product of the proto-oncogene.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D illustrate components of the three dimensional (3D) regulatory landscape. FIG. 1A (left panel) shows enhancers, insulators and insulated neighborhoods. Enhancers are occupied by transcription factors, mediator and cohesin, and their associated nucleosomes are acetylated at histone H3 on lysine 27. Candidate insulators are occupied by CTCF and cohcsin. FIG. 1A (right panel) shows a model of insulated neighborhoods formed by cohesin-associated CTCF-CTCF interactions, within which enhancers loop to promoters of target genes. FIG. 1B is a heatmap representation of ChIP-seq data for H3K27ac, MED1, OCT4, CTCF and H3K27me3 at SMC1-occupied regions in naive (left panel) and primed (right panel) hESCs. Read density is displayed within a 10 kb window and color scale intensities are shown in rpm/bp. Cohesin occupies three classes of sites: enhancer-promoter sites, polycomb-occupied sites, and CTCF-occupied sites. FIG. 1C shows cohesin (SMC1) ChIA-PET data analysis at the MYCN locus in naive hESCs. The algorithm used to identify paired-end tags (PETs) is described herein. PETs and interactions involving enhancers and promoters within the window are displayed at each step in the analysis pipeline: unique PETs, PET peaks and high-confidence interactions supported by at least 3 independent PETs and with a FDR of 0.01. Binding profiles for CTCF, SMC1 and H3K27ac are displayed at the bottom. FIG. 1D shows high-confidence cohesin-associated interaction maps in naive (left panel) and primed (right panel) hESCs. CTCF binding sites, enhancers and promoters involved in cohesin-mediated interactions are indicated as circles, and the size of circles correspond to the number of sites. The interactions between two regions are indicated as gray lines, and the size of lines correspond to the number of interactions.

FIGS. 2A-2E demonstrate that CTCF-CTCF/cohesin loops underlie much of TAD structure. FIG. 2A represents a heatmap of cohesin-associated CTCF-CTCF loops showing that these loops in naive hESCs are largely preserved in primed hESCs. The color bar indicates normalized ChIA-PET signal per loop. The 12,987 CTCF-CTCF/cohesin loops in naive hESCs were ranked by size and shown when present in primed hESCs. FIG. 2B presents CTCF motif orientation analysis of CTCF-CTCF loops. The percentage of each type of CTCF motif orientation is shown in a bar graph. FIG. 2C represents a TAD heat map of interaction frequencies and CTCF-CTCF loops. Normalized Hi-C interaction frequencies in H1 hESCs are displayed in a two-dimensional heat map (Dixon et al., 2015) with the TADs indicated as black bars. CTCF-CTCF/cohesin loops are indicated as blue lines (naive) and red lines (primed). A correlation analysis between Hi-C interaction frequency (H1 hESCs) and CTCF-CTCF/cohesin loops in naive and primed hESCs is displayed to the right in a boxplot; randomly generated TADs were used as the background control. FIG. 2D illustrates that CTCF-CTCF loops span many TADs identified using Hi-C data in HI hESCs. Chromosome 6 is displayed as a circos plot in both naive and primed hESCs, with zoomed in regions below. CTCF-CTCF loops (≥1 PETs) are indicated as blue arcs (naive) and red arcs (primed). The bar graphs show percentages of TADs spanned by CTCF-CTCF loops when various confidence thresholds (1, 2, ≥3 PETs) were used. As the background control, we used random shuffling of TAD locations (100 iterations). FIG. 2E shows Cohesin ChIA-PET data can be used to discover TADs. A comparison of TADs derived with the same algorithm from Hi-C data (Dixon et al., 2015) and cohesin ChIA-PET data for a portion of chromosome 12 (left panel). A global analysis indicates that TADs derived with the cohesin ChIA-PET data have boundaries that occur near those of TADs derived boundaries derived from Hi-C data in H1 hESCs (right panel).

FIGS. 3A-3D show putative insulated neighborhoods in hESCs. FIG. 3A shows a model of insulated neighborhood. FIG. 3B illustrates enhancer-promoter interactions occur predominantly within CTCF-CTCF loops in hESCs. The color bar indicates normalized high confidence interactions per loop. FIG. 3C demonstrates that CTCF-CTCF loops tend to be preserved in syntenic regions of human and mouse ESCs. Heatmaps of Hi-C interaction frequencies in H1 hESCs (upper panel) or mESCs (lower panel) are displayed to illustrate a syntenic region (human chr12: 91760000-94960000, mouse chr10: 94080000-96800000). FIG. 3D shows multiple insulated neighborhoods in mESCs whose CTCF boundaries were previously shown to be necessary for insulator function are preserved in human ESCs. The scissor-marked regions were deleted by CRISPR/Cas9 editing in mESCs, which caused local mis-regulation of gene expression (Dowen et al., 2014).

FIGS. 4A-4F shows 3D regulatory structures of TADs containing key pluripotency genes. In particular, FIGS. 4A-4F illustrate models of 3D structure for TADs containing SMAD3, HMGB3, TBX3, LEFTY1, KLF4 and NANOG, respectively, in naive hESCs. For each TAD, Hi-C interaction data (Dixon et al., 2015) is shown together with cohcsin-associated loop data for TAD-spanning CTCF loops, insulated neighborhood-spanning CTCF loops, enhancer-enhancer loops and enhancer-promoter loops. A subset of CTCF-CTCF loops was selected for display based on a directionality index (Extended Experimental Procedures) and a subset of genes present in these loops is shown for simplicity.

FIGS. 5A-5G show that differential enhancer landscape reveals key transcription factors, chromatin regulators and miRNAs in naive and primed pluripotency. FIG. 5A shows a scatterplot comparison of H3K27ac ChIP-seq peaks used to call enhancers in naive and primed hESCs. FIG. 5B shows a scatterplot comparison of super-enhancers in naive and primed hESCs. FIG. 5C shows the distribution of differential H3K27ac ChIP-seq signal density across the super-enhancer regions of naive and primed hESCs. Genes encoding key transcription factors, chromatin regulators, and miRNAs associated with super-enhancers are listed. FIG. 5D shows the 3D regulatory structure of a TAD containing TBX3 in both naive and primed hESCs with Hi-C and cohesin ChIA-PET data as described in FIG. 4. The naive and primed cells share TAD and insulated neighborhood structure, but a super-enhancer is present and loops to the TBX3 promoter only in naive cells. FIG. 5E shows the 3D regulatory structure of a TAD containing OTX2 in both naive and primed hESCs with Hi-C and cohesin ChIA-PET data as described in FIG. 4. The naive and primed cells share TAD and insulated neighborhood structure, but multiple super-enhancers are present and there is evidence for looping to the OTX2 promoter only in primed cells. FIG. 5F shows CTCF binding to the TAD and insulated neighborhood (IN) anchor sites is preserved in a broad spectrum of human cell types in the domain containing TBX3. FIG. 5G shows CTCF binding to the TAD and insulated neighborhood (IN) anchor sites is preserved in a broad spectrum of human cell types in the domain containing OTX2.

FIGS. 6A-6E depict conservation of CTCF sites used in loop anchors in hESCs and disease-associated variation. FIG. 6A shows DNA sequence in anchor regions of CTCF-CTCF loops in hESCs is far more conserved in primates than DNA sequence in hESC regions bound by CTCF that do not serve as loop anchors. FIG. 6B shows CTCF DNA sequence motif in anchor regions of CTCF-CTCF loops in hESCs is far more conserved in primates than CTCF DNA sequence motif in hESC regions bound by CTCF that do not serve as loop anchors. The CTCF sequence motif at sites used to anchor DNA loops in hESCs is far more conserved in primates than that motif at sites that do not serve as loop anchors in hESCs. FIG. 6C shows a CTCF-CTCF loop containing the PAX3 gene in human and ChIP-seq gene tracks showing conserved binding of CTCF at this locus in Orangutan, Chimpanzee and Tamarin genomes (Schwalie et al., 2013). FIG. 6D illustrates a catalog of SNPs linked to phenotypic traits and diseases in genome-wide association studies (GWAS) and SNP association with enhancer and CTCF anchor regions in hESCs. (Left) Pie chart showing percentage of SNPs associated with the highlighted classes of traits and diseases. (Middle Left) Distribution of trait-associated SNPs in coding and noncoding regions of the genome. (Middle Right) Location of all noncoding trait-associated SNPs relative to all enhancers identified in 86 human cell and tissue samples. x axis reflects binned distances of each SNP to the nearest enhancer. SNPs located within enhancers are assigned to the 0 bin. (Right) Location of all noncoding trait-associated SNPs relative to CTCF binding sites in loop anchor regions. FIG. 6E shows cancer mutations in transcription factor motifs at hESC CTCF-CTCF loop anchors.

FIGS. 7A-7F represent a three dimensional (3D) regulatory landscape of the T-ALL genome. FIG. 7A depicts models of the mechanisms activating proto-oncogenes in human cancer. FIG. 7B depicts a Circos plot of TADs in hESCs (H1) and CTCF binding sites, H3K27Ac binding sites, and cohesin ChIA-PET interactions in Jurkat cells on Chr21q. FIG. 7C shows a Hi-C interaction map and TADs defined using the Hi-C interaction frequency in hESC (H1), and cohcsin ChIA-PET interactions, CTCF ChIP-Seq and binding peaks, H3K27Ac (enhancer mark) ChIP-seq and binding peaks, RNA-Seq in Jurkat cells at the CD3D locus. Binding peaks are denoted as bars above binding profiles. FIG. 7D shows a summary of types of interactions in the Jurkat ChIA-PET data. FIG. 7E is a heat map of the density of ChIA-PET interactions around the 15,339 CTCF-CTCF interactions. The CTCF-CTCF interactions were length normalized. FIG. 7F shows ChIA-PET interactions at the RUNX1 locus. A subset of the cohesin ChIA-PET interactions is displayed above the binding profiles of CTCF, cohesin (SMC1) and H3K27Ac.

FIGS. 8A-8C represent active oncogenes and silent proto-oncogenes in isolated neighborhoods. FIG. 8A shows a list of genes implicated in T-ALL pathogenesis (T-ALL Pathogenesis Genes). Colored boxes indicate whether a gene is located within a neighborhood, expressed (per RNA-Seq) and associated with a super-enhancer. FIG. 8B shows an insulated neighborhood at the active TAL1 locus. The cohesin ChIA-PET interactions are displayed above the binding profiles of CTCF, cohesin (SMC1) H3K27Ac, and RNA-Seq track. A model of the insulated neighborhood surrounding the locus is shown on the right. FIG. 8C shows an insulated neighborhood at the silent LMO2 locus.

FIGS. 9A-9H depict a disruption of insulated neighborhood boundaries linked to proto-oncogene. FIG. 9A depicts and insulated neighborhood at the TAL1 locus in Jurkat T-ALL cells. A subset of cohesin ChIA-PET interactions is displayed above the ChIP-Seq binding profiles of CTCF and cohesin (SMC1). Patient deletions described in J. Zhang et al., Nature Genet., June 2013, 45:602 are shown as bars below the gene models. The deletion on the bottom indicates the recurrently deleted region identified in C. G. Mullighan et al., Nature, 12 Apr. 2007, 446:758. FIG. 9B shows ChIP-Seq binding profiles of CTCF, H3K27Ac, p300 and CBP, and RNA-Seq at the TAL1 locus in HEK-293T cells. The region deleted using a CRTSPR/Cas9-based approach is highlighted in a grey box. FIG. 9C shows a qRT-PCR analysis of TAL1 expression in wild type HEK-293T cells (wt), and in cells where the neighborhood boundary highlighted on (D) was deleted. Data is from two independent biological replicates; P<0.01 between wt and boundary-deleted cells (two-tailed t-test). FIG. 9D is a schematic model of the neighborhood organization and perturbation at the TAL1 locus. (E=enhancer). FIG. 9E depicts an insulated neighborhood at the LMO2 locus in Jurkat T-ALL cells. A subset of cohesin ChIA-PET interactions is displayed above the ChIP-Seq binding profiles of CTCF and cohesin (SMC1). Patient deletions described in J. Zhang et al., Nature Genet., June 2013, 45:602 are shown as bars below the gene models. The short deletion on the bottom indicates the recurrently deleted region identified in C. G. Mullighan et al., Nature, 12 Apr. 2007, 446:758. FIG. 9F is a ChIP-Seq binding profile of CTCF and H3K27Ac, p300 and CBP, and RNA-Seq at the LMO2 locus in HEK-293T cells. The region deleted by a CRISPR/Cas9-based approach is highlighted in a grey box. FIG. 9G is a qRT-PCR analysis of LMO2 expression in wild type HEK-293T cells (wt), and in cells where the neighborhood boundary highlighted on (H) was deleted. Data is from two independent biological replicates; P<0.05 between wt and boundary-deleted cells (two-tailed t-test). FIG. 9H is a schematic model of the neighborhood organization and perturbation at the LMO2 locus.

FIGS. 10A-10C refer to microdeletions that disrupt neighborhood boundaries in many cancers. FIG. 10A is an example of a “proto-oncogene neighborhood” at the NOTCH1 locus. CTCF ChIP-Seq and Cohesin ChIA-PET interactions in Jurkat (T-ALL), GM12878 (lymphoblastoid) and K562 (CML) cells are displayed. FIG. 10B represents proto-oncogene neighborhoods whose boundary is overlapped by at least one deletion in the COSMIC database. The bar chart depicts the number of cancer types in which the deletions occur. FIG. 10C shows examples of chromosomal deletions overlapping proto-oncogene neighborhood boundaries at six loci. Proto-oncogenes are highlighted in red. The chromosomal deletions are denoted as a red bar below the gene models.

FIGS. 11A-11E show human ESCs, expression analysis and ChIA-PET data. FIG. 11A show phase and fluorescence images of primed hESCs (endogenous OCT4-2A-GFP) and emerging naive colonies induced by treating these primed hESCs with 5i/L/A medium for 10 days. 40× magnification. FIG. 11B shows cross-species hierarchical clustering of expression datasets from naive and primed pluripotent cells in both mouse and human highlights the similarity of our datasets to the existing datasets for these cell states in human and mouse samples. FIG. 11C depict a comparison between the transcriptomes of naive and primed hESCs reveals common and differentially expressed genes. FIG. 11D illustrates a correlation analysis for two replicates of cohesin ChIA-PET dataset were displayed by scatter plot. FIG. 11E depict a percentage of cohesin ChIA-PET interactions that overlap in replicates in naive and primed hESCs.

FIGS. 12A-12C illustrate that cohesin-mediated interactions are largely responsible for the organization of TADs. FIG. 12A shows a saturation analysis for the cohesin ChIA-PET datasets in naive (left panel) and primed (right panel) hESCs. FIG. 12B shows that CTCF-CTCF loops span many TADs identified using Hi-C data in mESCs. Chromosome 2 is displayed as a circos plot in mESCs, with a zoomed in region below. CTCF-CTCF loops (≥1 PETs) are indicated as purple arcs. The bar graphs show percentages of TADs spanned by CTCF-CTCF loops when various confidence thresholds (1, 2, ≥3 PETs) were used. As a background control, we used random shuffling of TAD locations (100 iterations). FIG. 12C illustrates that Cohcsin ChIA-PET data can be used to discover TADs in mESCs. A comparison of TADs derived with the same algorithm from Hi-C data in mESCs (Dixon et al., 2012) and cohesin ChIA-PET data (mESCs) for a portion of chromosome 6.

FIG. 13 shows cohesin ChIA-PET interactions. In particular, FIG. 13 is a heatmap showing that cohesin ChIA-PET interactions occur predominantly within CTCF-CTCF loops. The color bar indicates normalized high confidence interactions per loop.

FIGS. 14A-14D show 3D structures of TADs containing key pluripotency genes in naive and primed hESCs. In particular, FIGS. 14A-14D represents models of 3D structure for TADs containing NANOG, PRDM14, SOX2 and OCT4, respectively, in naive and primed hESCs. For each TAD, Hi-C interaction data (Dixon et al., 2015) is shown together with cohesin-associated loop data for TAD-spanning CTCF loops and insulated neighborhood spanning CTCF loops. A subset of CTCF-CTCF loops was selected for display based on a directionality index (Extended Experimental Procedures) and a subset of genes present in these loops is shown for simplicity.

FIGS. 15A-15D show that differential regulated genes occur in 3D regulatory structures of TADs in naive and primed hESCs. In particular, FIGS. 15A-15D represent models of 3D structure for TADs containing KLF4, HMGB3, DUSP6 and TCF4, respectively, in naive and primed hESCs. For each TAD, Hi-C interaction data (Dixon et al., 2015) is shown together with cohesin associated loop data for TAD-spanning CTCF loops, insulated neighborhood spanning CTCF loops, enhancer-enhancer loops and enhancer-promoter loops. A subset of CTCF-CTCF loops was selected for display based on a directionality index and a subset of genes present in these loops is shown for simplicity.

FIGS. 16A-16B show conservation of hESC CTCF loop anchors. FIG. 16A shows that the DNA sequence in anchor regions of CTCF-CTCF loops in hESCs is far more conserved in vertebrates than DNA sequence in hESC regions bound by CTCF that do not serve as loop anchors. FIG. 16B shows that CTCF DNA sequence motif in anchor regions of CTCF-CTCF loops in hESCs is far more conserved in vertebrates than CTCF DNA sequence motif in hESC regions bound by CTCF that do not serve as loop anchors. The CTCF sequence motif at sites used to anchor DNA loops in hESCs is far more conserved in vertebrates than that motif at sites that do not serve as loop anchors in hESCs.

FIGS. 17A-17D are directed to cohesin ChIA-PET processing and analysis data. FIG. 17A shows a model of the hierarchical organization of chromosome structures. FIG. 17B is a heatmap representation of ChIP-seq data for SMC1 (cohesin), MYB, RUNX1, GATA3, TALL RNAPII, H3K27Ac and CTCF at 44,094 SMC1-bound sites in Jurkat cells. The regions are centered on the summit of the binding peak, and read density is displayed within a 10 kb window. Color scale intensities are shown below the heatmaps in rpm/bp. The majority of cohesin-bound sites are co-bound by CTCF or H3K27Ac-marked enhancers in Jurkat cells. FIG. 17C is cohesin ChIA-PET data analysis at the RUNX1 locus. The algorithm used to identify paired-end tags (PETs) is described in detail in the Materials and Methods section. PETs and interactions involving enhancers, promoters and CTCF-bound sites within the window are displayed at each step in the analysis pipeline: unique PETs, PET peaks, interactions between PET peaks supported by at least three independent PETs and with a false positive likelihood of <1% (see Materials and Methods). FIG. 17D is a summary of the major classes of interactions identified in the cohesin ChIA-PET data Enhancers, promoters, and CTCF sites where interactions occur are displayed as blue circles, and the size of the circle is proportional to the number of regions. The interactions between two sites are displayed as gray lines, and the thickness of the gray line is proportional to the number of interactions. Note that in this analysis the CTCF sites displayed include only the non-enhancer, non-promoter CTCF sites.

FIGS. 18A-18F present data for cohesin ChIA-PET interactions. FIG. 18A is a scatter plot shows the number of uniquely mapped PETs per 40 kb bins of the genome in each dataset replicate. Each replicate was normalized to the total number of uniquely mapped PETs detected in that dataset. The ChIA-PET replicate datasets display high correlation. FIG. 18B is a bar graph showing the percentage of interactions from one replicate of the SMC1 ChIA-PET that are supported by at least one unique PET in the other replicate. FIG. 18C is saturation analysis of the merged ChIA-PET dataset. Subsampling of various fractions of PETs within the merged ChIA-PET dataset was performed, and the number of unique genomic positions of intrachromosomal PETs beyond the self-ligation distance cutoff of 5 kb was plotted. The solid line depicting the single exponential fitting of the data suggests ˜50% saturation. The top dashed line indicates the estimated 100% saturation. FIG. 18D is a pie chart showing the percentage of intrachromosomal and interchromosomal interactions in the merged ChIA-PET dataset. FIG. 18E is a pie chart showing the percentage of interactions that cross or do not cross TAD boundaries (defined in H1 human ESCs). FIG. 18F is the percentage of CTCF-CTCF interactions that show the motif orientation (purple arrow) indicated on the left. The CTCF binding motif is also displayed. The orientation of CTCF motifs at pairs of CTCF sites connected by cohesin ChIA-PET interaction is mostly convergent.

FIGS. 19A and 19B illustrate examples of active oncogenes and silent proto-oncogenes that occur in insulated neighborhoods in T-ALL. FIG. 19A shows examples of insulated neighborhoods containing active oncogenes at the GATA3, MYB and LMO1 loci in Jurkat cells. The cohesin ChIA-PET interactions are displayed above the binding profiles of CTCF, SMC1 (cohesin), H3K27Ac, and RNA-Seq track. Gene models are displayed below the binding profiles. FIG. 19B shows examples of insulated neighborhoods containing silent proto-oncogenes at the TLX1, OLIG2 and TLX3 loci in Jurkat cells. The cohesin ChIA-PET interactions are displayed above the binding profiles of CTCF, SMC1 (cohesin), H3K27Ac, and RNA-Seq track. Gene models are displayed below the binding profiles. On the middle panel, the T-ALL census gene is OLIG2.

FIG. 20 illustrates a distribution plot of the lengths of recurrent genomic deletions found in T-ALL genomes. Only deletions <500 kb in size are plotted.

FIGS. 21A-21F report data on the comparison of CTCF and SMC1 binding and cohesin ChIA-PET interactions in Jurkat, GM12878 and K562 cells. FIG. 21A depicts an overlap analysis of CTCF ChIP-Seq binding peaks in Jurkat, GM12878 and K562 cells. FIG. 21B shows an overlap analysis of Cohesin (SMC1 in Jurkat or RAD21 in GM12878 and K562) ChIP-Seq binding peaks in Jurkat, GM12878 and K562 cells. FIG. 21C depicts an overlap analysis of CTCF-CTCF/cohesin ChIA-PET interactions in Jurkat, GM12878 and K562 cells. FIG. 21D shows a distribution plot of the lengths of somatic genomic deletions found in the COSMIC database. Only deletions <500 kb in size are plotted. FIG. 21E indicates the percentage of deletions that overlap proto-oncogene neighborhood boundaries (left) and constitutive CTCF-CTCF loops (right) in cancer types annotated in the COSMIC database. The number of deletions that overlap a proto-oncogene neighborhood boundary or CTCF-CTCF loop and the total number of deletions annotated in the respective cancer types are highlighted at the radar circumference. Plotted on the radii is the percentage of deletions overlapping at least one proto-oncogene neighborhood or constitutive CTCF-CTCF loop of the total number of deletions annotated in the respective cancer type. FIG. 21F represents a table of references supporting the example proto-oncogenes whose neighborhood is disrupted by a deletion (displayed on FIG. 10C) being activated in the cancer types the deletion was documented in.

FIGS. 22A-22L illustrate disruption of insulated neighborhood boundaries is linked to proto-oncogene activation. FIG. 22A depicts cohesin ChIA-PET interactions, ChIP-Seq profiles of CTCF, H3K27Ac, and RNA-Seq at the NTSR1 locus in HEK-293T cells. The CTCF boundary site frequently mutated in the ICGC database is highlighted by an arrow. The region deleted using a CRISPR/Cas9-based approach is highlighted in a grey box. FIG. 22B provides qRT-PCR analysis of NTSR1 expression in wild type HEK-293T cells (wt), and in cells where the neighborhood boundary highlighted on (A) was deleted. FIG. 22C is a model of the neighborhood and perturbation at the NTSR1 locus. FIG. 22D depicts cohesin ChIA-PET interactions, ChIP-Seq profiles of CTCF, H3K27Ac, and RNA-Seq at the WNT8B locus in HEK-293T cells. The CTCF boundary site frequently mutated in the ICGC database is highlighted by an arrow. The region deleted using a CRISPR/Cas9-based approach is highlighted in a grey box. FIG. 22E provides qRT-PCR analysis of WNT8B expression in wild type HEK-293T cells (wt), and in cells where the neighborhood boundary highlighted on (D) was deleted. FIG. 22F is a model of the neighborhood and perturbation at the WNT8B locus. FIG. 22G depicts cohesin ChIA-PET interactions, ChIP-Seq profiles of CTCF, H3K27Ac, and RNA-Seq at the BNC1 locus in HEK-293T cells. The CTCF boundary site frequently mutated in the ICGC database is highlighted by an arrow. The region deleted using a CRISPR/Cas9-based approach is highlighted in a grey box. FIG. 22H provides qRT-PCR analysis of BNC1 expression in wild type HEK-293T cells (wt), and in cells where the neighborhood boundary highlighted on (G) was deleted. FIG. 22I is a model of the neighborhood and perturbation at the BNC1 locus. FIG. 22J depicts cohesin ChIA-PET interactions, ChIP-Seq profiles of CTCF, H3K27Ac, and RNA-Seq at the PLP1 locus in HEK-293T cells. The CTCF boundary site frequently mutated in the ICGC database is highlighted by an arrow. The region deleted using a CRISPR/Cas9-based approach is highlighted in a grey box. FIG. 22K provides qRT-PCR analysis of PLP1 expression in wild type HEK-293T cells (wt), and in cells where the neighborhood boundary highlighted on (J) was deleted. FIG. 22L is a model of the neighborhood and perturbation at the PLP1 locus.

The following Tables S1-S19 are submitted herewith as Appendices 1-19, respectively. The Tables referenced herein were previously submitted in U.S. Provisional Application No. 62/195,559, and are hereby incorporated by reference in their entirety.

Table S1—Summary statistics of the Jurkat SMC1 ChIA-PET data;

Table S2—SMC1 ChIA-PET interactions;

Table S3—T-ALL Pathogenesis Genes;

Table S4—Gene expression (RPKM) values in Jurkat cells;

Table S5—Deletions in T-ALL genomes overlapping insulated neighborhood boundaries;

Table S6—Constitutive neighborhoods across three cell types;

Table S7—Candidate proto-oncogenes extracted from the Cancer Gene Census;

Table S8—Proto-oncogene neighborhoods;

Table S9—Somatic deletions in cancer genomes (COSMIC) overlapping constitutive CTCF-CTCF loop boundaries;

Table S10—Somatic deletions in cancer genomes (COSMIC) overlapping proto-oncogene neighborhood boundaries;

Table S11—GEO accession IDs of the datasets used in Example 2;

Table S12—RNA-seq gene expression in naive and primed hESCs;

Table S13—SMC1 ChIA-PET peaks for hESCs;

Table S14—H3K27ac ChIP-seq peaks for hESCs;

Table S15—CTCF ChIP-seq peaks for hESCs;

Table S16—High confidence SMC1 ChIA-PET interactions for naive hESCs;

Table S17—High confidence SMC1 ChIA-PET interactions for primed hESCs;

Table S18—Differential super-enhancer associated transcription factors, chromatin regulators and miRNAs in naive and primed hESCs; and

Table S19—Cancer mutations occur in CTCF loop anchors.

The material submitted herewith in electronic (.txt) form and comprising Appendices 1-19 (Tables S1-S19, respectively) is incorporated herein by reference, specifically:

Appendix 1 (file name: S1.txt; date created: Jul. 14, 2016 and file size: 1765 bytes);

Appendix 2 (file name: S2.txt; date created: Jul. 14, 2016 and file size: 1169729 bytes);

Appendix 3 (file name: S3.txt; date created: Jul. 14, 2016 and file size: 1314 bytes);

Appendix 4 (file name: S4.txt; date created: Jul. 14, 2016 and file size: 1652641 bytes);

Appendix 5 (file name: 55.txt; date created: Jul. 14, 2016 and file size: 8379 bytes);

Appendix 6 (file name: S6.txt; date created: Jul. 14, 2016 and file size: 750491 bytes);

Appendix 7 (file name: S7.txt; date created: Jul. 14, 2016 and file size: 7271 bytes);

Appendix 8 (file name: 58.txt; date created: Jul. 14, 2016 and file size: 16218 bytes);

Appendix 9 (file name: S9.txt; date created: Jul. 14, 2016 and file size: 128354 bytes);

Appendix 10 (file name: S10.txt; date created: Jul. 14, 2016 and file size: 13540 bytes);

Appendix 11 (file name: S11.txt; date created: Jul. 14, 2016 and file size: 1607 bytes);

Appendix 12 (file name: S12.txt; date created: Jul. 14, 2016 and file size: 2726366 bytes);

Appendix 13 (file name: S13.txt; date created: Jul. 14, 2016 and file size: 5571145 bytes);

Appendix 14 (file name: S14.txt; date created: Jul. 14, 2016 and file size: 2322928 bytes);

Appendix 15 (file name: S15.txt; date created: Jul. 14, 2016 and file size: 2272070 bytes);

Appendix 16 (file name: S16.txt; date created: Jul. 14, 2016 and file size: 4237674 bytes);

Appendix 17 (file name: S17.txt; date created: Jul. 14, 2016 and file size: 5694412 bytes);

Appendix 18 (file name: S18.txt; date created: Jul. 14, 2016 and file size: 575884 bytes); and

Appendix 19 (file name: S19.txt; date created: Jul. 14, 2016 and file size: 1204141 bytes).

### DETAILED DESCRIPTION OF THE INVENTION

The gene expression programs that establish and maintain specific cell states in humans are controlled by regulatory proteins that bind specific genomic elements (Heinz et al., 2015; Levine et al., 2014; Plank and Dean, 2014; Shlyueva et al., 2014; Smallwood and Ren, 2013; Smith and Shilatifard, 2014; Spitz and Furlong, 2012) Enhancer elements, first described over 30 years ago (Banerji et al., 1981; Benoist and Chambon, 1981; Gruss et al., 1981), are bound by transcription factors and can loop long distances to contact and regulate specific genes. There are approximately 1 million enhancers that have been identified in the human genome (Dunham et al., 2012; Thurman et al., 2012), and the constraints that cause them to operate only on their specific target genes are not well understood. Insulator elements are bound by CTCF and prevent enhancers from operating across insulator boundaries (Bell et al., 1999; Cai and Levine, 1995; Geyer and Corces, 1992) and recent studies suggest such boundaries function in the context of 3D chromosome structures (DeMare et al., 2013; Dixon et al., 2012; Dowen et al., 2014; Handoko et al., 2011; Heidari et al., 2014; Kieffer-Kwon et al., 2013; Nora et al., 2012; Phillips-Cremins et al., 2013; Rao et al., 2014; Sanyal et al., 2012). Thus the global arrangement of enhancers and insulators provides regulatory and structural features that enable the control of each cell's gene expression program.

The mammalian genome is organized in a 3D topology that is thought to contribute to the regulation of gene expression, in part by creating constraints that produce regions of active and repressed transcription (Bickmore, 2013; de Graaf and van Steensel, 2013; de Laat and Duboule, 2013; Gibcus and Dekker, 2012; Gorkin et al., 2014; Pombo and Dillon, 2015). Recent evidence indicates that both active and repressed compartments of chromosomes are partitioned into megabase-size topologically associating domains (TADs) (Dixon et al., 2012; Lieberman-Aiden et al., 2009; Nora et al., 2012). TADs are regions of chromosomes that show evidence of relatively high DNA interaction frequencies based on Hi-C chromosome conformation capture data and are characterized by boundaries that delimit the range of local intra-chromosomal interactions. TADs appear to provide structural constraints that limit the ability of regulatory elements such as enhancers to contact and function at specific target genes within TADs. TADs are largely maintained through development, as TAD boundaries tend to be similar among various cell types, which suggests that TADs are a fundamental unit of chromatin-mediated gene regulation in all cells (Dixon et al., 2015; Dixon et al., 2012; Phillips-Cremins et al., 2013). The chromosome-structuring proteins CTCF and cohesin have been shown to be important for the integrity of TAD boundaries and substructures (Dowen et al., 2014; Lupianez et al., 2015; Narendra et al., 2015; Phillips-Cremins et al., 2013; Seitan et al., 2013; Zuin et al., 2014).

CTCF and cohesin are essential for early embryogenesis, ubiquitously expressed and retained on their interphase chromatin sites in mitotic chromatin and are thus thought to play important roles in epigenetic inheritance (Dorsett and Merkenschlager, 2013; Gomez-Diaz and Corees, 2014; Jeppsson et al., 2014; Merkenschlager and Odom, 2013; Remeseiro and Losada, 2013). CTCF is an 11 zinc-finger protein that binds CCTC motifs and can form homodimers, enabling two distal DNA-bound CTCF molecules to loop DNA. Cohesin is loaded at enhancer-promoter loops and occupies these sites and CTCF sites (Dowen et al., 2013; Dowen et al., 2014; Hadjur et al., 2009; Kagey et al., 2010; Nativio et al., 2009; Parelho et al., 2008; Rubio et al., 2008; Schmidt et al., 2012; Sofueva et al., 2013; Wendt et al., 2008). Cohesin forms a large ring capable of encircling two DNA molecules and is thought to facilitate establishment and/or maintenance of enhancer-promoter loops and CTCF-CTCF loops. An emerging model suggests that cohesin-associated CTCF-CTCF loops occur within TADs and that enhancers generally interact with genes that occur within these loops (DeMare et al., 2013; Dixon et al., 2015; Dowen et al., 2014; Handoko et al., 2011; Heidari et al., 2014; Kieffer-Kwon et al., 2013; Li et al., 2012; Phillips-Cremins et al., 2013; Rao et al., 2014). These CTCF-CTCF loops appear to function as insulated neighborhoods for gene regulation because the loss of either of the CTCF sites that close the loop can alter gene regulation within and immediately outside the loop (Dowen et al., 2014). Insulated neighborhood structures have been described for key pluripotency genes (Dowen et al., 2014), but the extent to which these structures account for the Hi-C DNA interactions used to define TADs is not clear.

Work described herein reveals 3D regulatory landscapes of hESCs representative of early human development. This work also demonstrates that cohesin-associated CTCF loops, and the cohesin-associate enhancer-promoter loops within them, dominate the organization of TADs. The CTCF-CTCF loops form a chromosomal scaffold of insulated neighborhoods that are largely preserved in vertebrates, and enhancer-promoter interactions occur within these neighborhoods. Genes are regulated in the context of conserved insulated neighborhood structures. Loss of neighborhood structures occurs frequently in cancer cells, and proto-oncogenes can be activated by genetic alterations that disrupt specific 3D chromosome structures.

In some embodiments, TADs are organized by cohesin-associated loops (e.g., cohesin-associated CTCF-CTCF loops). A CTCF-CTCF loop forms an insulated neighborhood. In some embodiments, multiple CTCF-CTCF loops may be nested within one another. In some embodiments, a CTCF-CTCF loop may have a median length of at least 200 kb, or in some embodiments about 240 kb. In some embodiments, genes are located within the CTCF-CTCF loops.

The CTCF-CTCF loop may contain at least one gene (e.g., a target gene), or in some embodiments, may contain two to three genes. In some embodiments, CTCF-CTCF loops may be nested such that genes may be embedded within two or more independent CTCF-CTCF loops. The genes located within the CTCF-CTCF loops may be oncogenes or proto-oncogenes. In some embodiments, the CTCF-CTCF loops may include one or more of active oncogenes and silent proto-oncogenes.

In some embodiments, enhancers located outside and in proximity to the CTCF-CTCF loops may interact with the genes, for example with a target gene, located within the insulated neighborhood. In some aspects, an enhancer may be located within a CTCF-CTCF loop separate from the CTCF-CTCF loop where the target gene is located. The insulated neighborhoods may constrain interactions between regulatory elements and the genes located within the neighborhoods.

In some embodiments, a boundary of the insulated neighborhood may be disrupted. In some embodiments, the disruption may be a deletion (e.g., a microdeletion), a mutation, or some other disruption. The disruption of the insulated neighborhood boundary may affect the interaction between regulatory elements and the genes located within the neighborhoods. In some embodiments, the disruption of the insulated neighborhood boundary may play a role in the misregulation of gene expression that is inherent to a cancer state. Disruptions that overlap the isolated neighborhood boundaries may cause transcriptional activation of genes (e.g., proto-oncogenes) found within the CTCF-CTCF loops. In some embodiments, site-specific disruptions of the loop boundary CTCF site may activate the respective proto-oncogene in non-malignant cells.

In some embodiments, a method is provided of identifying one or more differences in a regulatory pathway between two cells comprising obtaining expression data for at least one enhancer from each cell from an insulated neighborhood conserved between the two cells and comparing said expression data to identify differential activity of said enhancer on at least one target gene. In some embodiments, said cells are embryonic stem cells. In some embodiments said cells are iPS cells. In some embodiments one cell is naive and one cell is primed. In some embodiments one cell is a more differentiated cell type than the other cell.

Also provided is a method for identifying a Topologically Associating Domain (TAD) comprising identifying TAD boundaries utilizing ChIA-PET data, e.g., cohesin ChIA-PET data, and identifying a TAD between two TAD boundaries.

Also provided is a method of inhibiting activation of a proto-oncogene by an enhancer, wherein one of the proto-oncogene or enhancer is located within an insulated neighborhood, comprising stabilizing the boundary of said insulated neighborhood such that disruption of the neighborhood is reduced, thereby inhibiting interaction of the enhancer with the proto-oncogene. In some embodiments the proto-oncogene is located within an insulated neighborhood. In some embodiments the enhancer is located within an insulated neighborhood. In some embodiments the enhancer and the proto-oncogene are each located within an insulated neighborhood, and wherein said insulated neighborhoods are different from one another.

Also provided is a method of identifying a super-enhancer in a 3D regulatory landscape of a cell comprising examining all enhancer activity within an insulated neighborhood, and stitching all enhancers located within the insulated neighborhood together to form a super-enhancer. In some aspects, genomic regions of DNA within the cell enriched for H3K27ac signal or Mediator signal are identified, and the enriched regions are stitched together if within 12.5 kb of each other. The stitched regions may be ranked by H3K27ac signal, and a ranked stitched regions is identified as a super-enhancer if the ranked stitched region falls above a threshold at which two classes of enhancers are separable.

As used herein, “enhancer” refers to a short region of DNA to which proteins (e.g., transcription factors) bind to enhance transcription of a gene. A super-enhancer comprises a genomic region of DNA that contains at least two enhancers. It should be appreciated that each of the at least two enhancers can be the same type of enhancer or the at least two enhancers can be different types of enhancers. Each enhancer of the at least two enhancers comprises a binding site for a cognate transcription factor that interacts with the transcriptional coactivator to stimulate transcription of the gene associated with the super-enhancer.

A super-enhancer comprises a genomic region of DNA that contains at least two enhancers, wherein the genomic region is occupied when present within a cell by more transcriptional coactivator (e.g., Mediator), more chromatin regulator (e.g., BRD4), and/or more RNA (e.g., eRNA) than the average single enhancer within the cell. In some embodiments, the genomic region of a super-enhancer is occupied when present within the cell by an order of magnitude more transcriptional coactivator, chromatin regulator, or RNA than the average single enhancer in the cell. As used herein, “order of magnitude” refers to the relative fold difference in a feature or classification of one object as compared to a feature or classification of another object (e.g., a level or an amount of transcriptional coactivator occupying a super-enhancer associated with a gene as compared to the level or the amount of transcriptional coactivator occupying the average or median enhancer associated with the gene). In some embodiments, the order of magnitude is at least 1-fold, 2-fold, 3-fold, 4-fold, 5-fold, 6-fold, 7-fold, 8-fold, 9-fold, 10-fold or more. In some embodiments, the order of magnitude is at least 2-fold (i.e., there is a 2-fold greater amount of transcriptional coactivator occupying the super-enhancer associated with a gene than the amount of transcriptional coactivator occupying the average enhancer in the gene). In some embodiments, the order of magnitude is at least 10-fold. In some embodiments, the order of magnitude is at least 15-fold. In some embodiments, the order of magnitude is at least 16-fold. It should be appreciated that any enhancer associated with a target gene can be cloned and used to form a super-enhancer.

As used herein, “transcriptional coactivator” refers to a protein or complex of proteins that interacts with transcription factors to stimulate transcription of a gene. In some embodiments, the transcriptional coactivator is Mediator. In some embodiments, the transcriptional coactivator is Med1 (Gene ID: 5469). In some embodiments, the transcriptional coactivator is a Mediator component. As used herein, “Mediator component” comprises or consists of a polypeptide whose amino acid sequence is identical to the amino acid sequence of a naturally occurring Mediator complex polypeptide. The naturally occurring Mediator complex polypeptide can be, e.g., any of the approximately 30 polypeptides found in a Mediator complex that occurs in a cell or is purified from a cell (see, e.g., Conaway et al., 2005; Kornberg, 2005; Malik and Roeder, 2005). In some embodiments a naturally occurring Mediator component is any of Med1-Med 31 or any naturally occurring Mediator polypeptide known in the art. In some embodiments, Mediator occupation of an enhancer, e.g., a super-enhancer, may be detected by detecting one or more Mediator components. It is to be understood that a Mediator inhibitor may inhibit one or more Mediator components or inhibit interaction(s) between them or inhibit interaction with a transcription factor.

Generally, super-enhancers formed by the at least two enhancers in the genomic region of DNA are of greater length than the average single enhancer. In some embodiments, the length of the genomic region that forms the super-enhancer is at least an order of magnitude greater than the average single enhancer. In some embodiments the genomic region spans between about 4 kilobases and about 500 kilobases in length. In some embodiments, the genomic region spans between about 4 kilobases and about 40 kilobases in length. It should be appreciated, however, that super-enhancers may comprise genomic regions less than 4 kilobases or greater than 40 kilobases in length, as long as the genomic region contains clusters of enhancers that can be occupied when present within a cell by extremely high levels of a transcriptional coactivator (e.g., Mediator).

Identifying super-enhancers within a cell and identifying a super-enhancer associated with a target gene can be achieved by a variety of different methods, as would be understood by a person skilled in the art. In some embodiments, the super-enhancer is identified by performing a high throughput sequencing method such as chromatin immunoprecipitation high-throughput sequencing (ChIP-Seq) or RNA-Seq. In some embodiments, the target gene associated with a super-enhancer may be identified by its proximity to the super-enhancer. In some aspects, the target gene may be a oncogene or a proto-oncogene.

In an embodiment, the gene is identified by selecting the nearest gene that meets a preselected criteria, e.g., the nearest expressed gene. In some embodiments, selection criteria can be defined based on RNA data (RNA-Seq, Gro-Seq, or microarray), or ChIP-Seq of transcription-associated signals (RNA polymerase II, H3K4me3, H3K27ac levels around the transcription start site). In an embodiment, the selection criteria comprises evaluation of transcription associated signals of H3K27ac using ChIP-Seq signal around the transcription start site of the genes to define the set of expressed genes in cells. In an embodiment, an expressed gene within a certain genomic window is selected. For example, in an embodiment a maximum distance between the super-enhancer center and the transcription start site of the regulated gene is set to evaluation of the gene.

In some embodiments, a super-enhancers presence may be identified using a probe. For example, a reaction mixture may comprise a probe that binds selectively to a super-enhancer component, e.g. binds selectively to a protein, e.g., Med1, H3K27ac, or a transcription factor, or to an eRNA. In embodiments the reaction mixture comprises a reagent capable of cross-linking, e.g., covalently cross-linking, nucleic acid, e.g., chromosomal or mitochondrial DNA, to a super-enhancer component. Exemplary super-enhancer components include a protein, e.g., Med1 or a transcription factor, or to an eRNA.

In some aspects, regions identified as being enhancer regions, were stitched together if within 12.5 kb of each other. In some aspects, the enhancer regions were pinpointed by identifying genomic regions of DNA enriched for H3K27ac signal, Mediator signal, or by identifying other potential known markers of an enhancer. Enriched regions entirely contained within +/−2 kb from a transcription start site were excluded from the stitching. Stitched regions may be ranked by the H3K27ac signal therein. ROSE identifies a point at which the two classes of enhancers are separable. Those stitched enhancers falling above this threshold may be considered super-enhancers. In some aspects, the stitching of the enhancer regions occurs linearly. In other aspects, the stitching of the enhancer regions occurs three-dimensionally. For example, all enhancer activity within a CTCF-CTCF loop may be considered when stitching regions together. Super-enhancers identified in a three-dimensional regulatory landscape may be larger than super-enhancers identified in a linear landscape.

Also provided is a method of identifying a disruption in an insulated neighborhood boundary comprising identifying at least one proto-oncogene of interest, identifying an insulated neighborhood within which the proto-oncogene is located (e.g., identifying candidate neighborhoods comprised of CTCF-CTCF loops wherein a transcription start site of the at least one proto-oncogene is located within the neighborhood), and examining the proto-oncogene neighborhood for disruptions, such as disruptions that overlap the proto-oncogene neighborhood boundary.

In some aspects, the proto-oncogene of interest is TAL1 or LMO2. In some aspects, an enhancer or super-enhancer may be located outside the insulated neighborhood within which the proto-oncogene of interest is located. The enhancer or super-enhancer may be located outside, but within proximity to the insulated neighborhood within which the proto-oncogene is located. In an embodiment, the enhancer or super-enhancer is located within an insulated neighborhood different the insulated neighborhood of the proto-oncogene.

In some aspects, a proto-oncogene of interest may be activated (e.g., become an oncogene). In one embodiment, the proto-oncogene of interest is activated by an enhancer or super-enhancer which is located outside the insulated neighborhood within which the proto-oncogene is located. In some embodiments, the activation of the proto-oncogene occurs because of a disruption in a proto-oncogene neighborhood boundary, i.e., in the absence of disruption of a neighborhood boundary the enhancer or super-enhancer does not activate the proto-oncogene. The disruption may be a deletion or a mutation in the boundary. In an embodiment, the disruption is a mutation in a CTCF-CTCF loop anchor region. In another embodiment, the disruption is a deletion or a microdeletion in a CTCF-CTCF loop anchor region. In some aspects, the disruption in the boundary may be a deletion and the proto-oncogene boundary may overlap the deletion by at least one base pair.

Also provided is a method of screening for cancer, comprising identifying a proto-oncogene of interest, wherein the proto-oncogene is located within an insulated neighborhood, examining the proto-oncogene insulated neighborhood for disruptions in a boundary of the proto-oncogene insulated neighborhood, and measuring expression of the proto-oncogene, wherein elevated levels of the proto-oncogene indicate a likelihood of cancer. In some aspects, an individual may be screened for a pre-disposition of cancer. The method for screening may comprise, identifying a proto-oncogene located within an insulated neighborhood, and determining if a boundary of the insulated neighborhood includes a disruption, wherein a disruption in the insulated neighborhood boundary indicates an increased risk of cancer.

In some embodiments, a disruption of the insulated neighborhood boundary is a deletion, mutation, or some other disruption. The disruption may occur as a deletion in a CTCF loop binding site. In some aspects, an enhancer is located within the vicinity or proximity, but outside, of the insulated neighborhood. A disruption of the insulated neighborhood boundary may be identified by determining if the enhancer has activated the proto-oncogene. In some embodiments, determining if proto-oncogenes located within insulated neighborhoods are activated via loss of a neighborhood boundary may occur by mapping insulated neighborhoods and other cis-regulatory interactions in a cancer cell genome using ChIA-PET.

As used herein, the term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythcmia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In certain aspects, the present invention relates to a method of treating a cancer involving an activated proto-oncogene, comprising administering to a patient in need of such treatment an effective amount of an agent that repairs a disruption (e.g., a deletion, mutation, or other disruption) in an insulated neighborhood boundary, wherein the activated proto-oncogene is located within the insulated neighborhood, thereby decreasing expression of the proto-oncogene such that the cancer is treated. In some aspects, a method of treating a cancer involving an activated proto-oncogene may include administering an effective amount of an agent that disrupts activation of a proto-oncogene, thereby decreasing expression of the proto-oncogene such that the cancer is treated.

Also provided is a method of identifying a candidate target for treating a cancer, comprising detecting a disrupted boundary of an insulated neighborhood that contains one or more proto-oncogenes in genomic DNA derived from the cancer, and identifying an enhancer located outside of and in proximity to the insulated neighborhood, thereby identifying the proto-oncogene and the enhancer as candidate targets for treating the cancer.

In some embodiments, the proto-oncogene is activated as a result of the disruption in the insulated neighborhood boundary. For example, a deletion in the neighborhood boundary may allow an enhancer or a super-enhancer to activate a proto-oncogene. In contrast, a proto-oncogene may not be expressed when the insulated neighborhood boundary is not disrupted. Expression of the proto-oncogene may be measured in a sample comprising cancer cells, wherein higher expression of the proto-oncogene in the cancer cells as compared to normal cells indicates that the proto-oncogene and/or the enhancer is a target for treating the cancer.

In some aspects, an agent may repair the disruption in the neighborhood boundary, thereby blocking the enhancer from activating the proto-oncogene. At least one additional agent may be administered to address the activated proto-oncogene and/or to treat a cancer. In some aspects, an agent may disrupt the activation of the proto-oncogene, and thereby decrease the expression of the proto-oncogene. In an embodiment, the agent may inhibit activity of the enhancer, inhibit expression of the proto-oncogene, and/or inhibit activity of a gene product of the proto-oncogene.

It should be appreciated that the present invention contemplates the use of any technique or any agent that is capable of disrupting the function of an enhancer or super-enhancer. Generally, disrupting the function of the super-enhancer involves contacting said super-enhancer region with an effective amount of an agent that interferes with occupancy of the super-enhancer region by a cognate transcription factor for the gene, a transcriptional coactivator, or a chromatin regulator. In some embodiments, disrupting the function of the super-enhancer can be achieved by contacting the super-enhancer region with a pause release agent. In certain embodiments, the agent interferes with a binding site on the super-enhancer for the cognate transcription factor, interferes with interaction between the cognate transcription factor and a transcriptional coactivator, inhibits the transcription coactivator, or interferes with or inhibits the chromatin regulator. In some embodiments, the agent is a bromodomain inhibitor. In some embodiments, the agent is a BRD4 inhibitor. In some embodiments, the agent is the compound JQI. In some embodiments, the agent is iBET.

Any of a wide variety of agents (also termed “compounds”) can be used to disrupt the function of the enhancer or super-enhancer, such as BET bromodomain inhibitors, P-TEFb inhibitors or compounds that interfere with binding of the cognate transcription factors to the binding sites of the super-enhancer associated with the gene (e.g, if the gene is an oncogene, such as MYC, a c-Myc inhibitor can be used to disrupt the function of a super-enhancer). An inhibitor could be any compound that, when contacted with a cell, results in decreased functional activity of a molecule or complex, e.g., transcriptional coactivator (e.g., Mediator), a chromatin regulator (e.g., BRD4), an elongation factor (e.g., P-TEFb), or cognate transcription factor (e.g., a cognate oncogenic transcription factor), in the cell. An inhibitor could act directly, e.g., by physically interacting with a molecule or complex to be inhibited, or a component thereof, or indirectly such as by interacting with a different molecule or complex required for activity of the molecule or complex to be inhibited, or by interfering with expression or localization.

It should be appreciated that the various agents described herein can be used alone, or in combination with other agents described, for example, an agent that interferes with c-Myc enhancer-driven transcription of a plurality of Myc target genes.

In some embodiments, an agent is administered in combination with a second therapeutic agent. In some embodiments, an agent is administered in combination with a cancer therapeutic agent. It should be appreciated that the combined administration of an agent of the present invention and a cancer therapeutic agent can be achieved by formulating the cancer therapeutic agent and agent in the same composition or by administering the cancer therapeutic agent and agent separately (e.g., before, after, or interspersed with doses or administration of the cancer therapeutic agent). In some embodiments, an agent of the present invention is administered to a patient undergoing conventional chemotherapy and/or radiotherapy. In some embodiments the cancer therapeutic agent is a chemotherapeutic agent. In some embodiments the cancer therapeutic agent is an immunotherapeutic agent. In some embodiments the cancer therapeutic agent is a radiotherapeutic agent.

Exemplary chemotherapeutic agents that can be administered in combination with the agents of the present invention (e.g., agents that repair disruptions in insulated neighborhoods, agents that disrupt or inhibit activation of proto-oncogenes, agents that disrupt or inhibit enhancer activity, and the like) include alkylating agents (e.g. cisplatin, carboplatin, oxaloplatin, mechlorethamine, cyclophosphamide, chorambucil, nitrosureas); anti-metabolites (e.g. methotrexate, pemetrexed, 6-mercaptopurine, dacarbazine, fludarabine, 5-fluorouracil, arabinosycytosine, capecitabine, gemcitabine, decitabine); plant alkaloids and terpenoids including vinca alkaloids (e.g. vincristine, vinblastine, vinorelbine), podophyllotoxin (e.g. etoposide, teniposide), taxanes (e.g. paclitaxel, docetaxel); topoisomerase inhibitors (e.g. notecan, topotecan, amasacrine, etoposide phosphate); antitumor antibiotics (dactinomycin, doxorubicin, epirubicin, and bleomycin); ribonucleotides reductase inhibitors; antimicrotubules agents; and retinoids. (See, e.g., Cancer: Principles and Practice of Oncology (V. T. DeVita, et al., eds., J.B. Lippincott Company, 9th ed., 2011; Brunton, L., et al. (eds.) Goodman and Gilman's The Pharmacological Basis of Therapeutics, 12th Ed., McGraw Hill, 2010).

Exemplary immunotherapeutic agents include cytokines, such as, for example interleukin-1 (IL-I), IL-2, IL-4, IL-5, IL-ß, IL-7, IL-10, IL-12, IL-15, IL-18, CSF-GM, CSF-G, IFN-γ, IFN-α, TNF, TGF-ß but not limited thereto.

In some embodiments an agent of the present invention can be linked or conjugated to a delivery vehicle, which may also contain cancer therapeutic. Suitable delivery vehicles include liposomes (Hughes et al. Cancer Res 49(22):6214-20, 1989, which is hereby incorporated by reference in its entirety), nanoparticles (Farokhzad et al. Proc Nat'l Acad Sci USA 103(16):6315-20, 2006, which is hereby incorporated by reference in its entirety), biodegradable microspheres, microparticles, and collagen minipellets. The delivery vehicle can contain any of the agents and/or compositions of the present invention, as well as chemotherapeutic, radiotherapeutic, or immunotherapeutic agents described supra.

In some embodiments an agent of the present invention can be conjugated to a liposome delivery vehicle (Sofou and Sgouros, Exp Opin Drug Deliv. 5(2):189-204, 2008, which is hereby incorporated by reference in its entirety). Liposomes are vesicles comprised of one or more concentrically ordered lipid bilayers which encapsulate an aqueous phase. Suitable liposomal delivery vehicles are apparent to those skilled in the art. Different types of liposomes can be prepared according to Bangham et al. J. Mol. Biol. 13:238-52, 1965; U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., which are hereby incorporated by reference in their entirety.

These liposomes can be produced such that they contain, in addition to the therapeutic agents of the present invention, other therapeutic agents, such as immunotherapeutic cytokines, which would then be released at the target site (e.g., Wolff et al., Biochim. Biophys. Acta. 802:259-73, 1984, which is hereby incorporated by reference in its entirety).

The present invention also contemplates a composition comprising an agent of the present invention and a pharmaceutically acceptable carrier, diluent, or excipient. Therapeutic formulations of the agents of the present invention can be prepared having the desired degree of purity with optional pharmaceutically acceptable carriers, excipients or stabilizers (REMINGTON'S PHARMACEUTICAL SCIENCES (A. Osol ed. 1980), which is hereby incorporated by reference in its entirety), in the form of lyophilized formulations or aqueous solutions. Acceptable carriers, excipients, or stabilizers are nontoxic to recipients at the dosages and concentrations employed, and include buffers such as acetate, Tris-phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG).

The active therapeutic ingredients of the pharmaceutical compositions alone or in combination with or linked to a cancer therapeutic agent or radiotherapeutic agent) can be entrapped in microcapsules prepared using coacervation techniques or by interfacial polymerization, e.g., hydroxymethylcellulose or gelatin-microcapsules and poly-(methylmethacylate) microcapsules, respectively, in colloidal drug delivery systems (e.g., liposomes, albumin microspheres, microemulsions, nano-particles and nanocapsules) or in macroemulsions. Such techniques are disclosed in REMINGTON'S PHARMACEUTICAL SCIENCES (A. Osol ed. 1980), which is hereby incorporated by reference in its entirety. In some embodiments the agents of the present invention can be conjugated to the microcapsule delivery vehicle to target the delivery of the therapeutic agent to the site of the cells exhibiting enhancer activated proto-oncogenes.

Sustained-release preparations may be prepared. Suitable examples of sustained-release preparations include semi-permeable matrices of solid hydrophobic polymers containing the antibody or polypeptide, which matrices are in the form of shaped articles, e.g., films or microcapsules. Examples of sustained-release matrices include polyesters, hydrogels (for example, poly(2-hydroxyethyl-methacrylate), or poly(vinylalcohol)), polylactides, copolymers of L-glutamic acid and .gamma. ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

In some embodiments, an agent of the present invention can be provided with an enteric coating or otherwise protected from hydrolysis or low stomach pH. The therapeutically effective compositions containing the agents of the present invention are administered to a subject, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes.

Other therapeutic regimens may be combined with the administration of the agents of the present invention. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect. In some embodiments, a composition of the present invention is administered in combination with a therapy selected from the group consisting of chemotherapy, radiotherapy, proton therapy, surgery, and combinations thereof.

The composition can include any number of additional active ingredients which can act in concert to provide a therapeutic effect, (e.g., a synergistic therapeutic effect), such as a chemotherapeutic agent, a radiotherapeutic agent, a nutritional supplement (e.g. vitamins), an antioxidant, and combinations thereof.

An “effective amount” or “effective dose” of an agent (or composition containing such agent) generally refers to the amount sufficient to achieve a desired biological and/or pharmacological effect, e.g., when contacted with a cell in vitro or administered to a subject according to a selected administration form, route, and/or schedule. As will be appreciated by those of ordinary skill in the art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or through use of multiple doses, in various embodiments. It will be understood that agents, compounds, and compositions herein may be employed in an amount effective to achieve a desired biological and/or therapeutic effect.

Also provided is a method of identifying an agent that stabilizes an insulated neighborhood, wherein the insulated neighborhood has a disrupted boundary (e.g., the boundary has a deletion, mutation, or other disruption), comprising transfecting a cell with a super-enhancer and the insulated neighborhood under conditions suitable for the super-enhancer to drive high levels of expression of a proto-oncogene that is associated with the super-enhancer and is located within the insulated neighborhood, contacting the cell with a test agent, and measuring the level of expression of the proto-oncogene, wherein decreased expression of the proto-oncogene in the presence of the test agent indicates that the test agent is an agent that stabilizes an insulated neighborhood. In some embodiments, a nucleic acid comprising the super-enhancer and/or insulated neighborhood is transfected into the cell. Methods of forming nucleic acid constructs are known to those skilled in the art. It should be understood that the nucleic acid constructs of the present invention are artificial or engineered constructs not to be confused with native genomic sequences. In an embodiment, the agent repairs a disruption in the disrupted insulated neighborhood boundary. In some aspects, expression of the proto-oncogene is measured, at least in part, by measuring the level of a gene product encoded by the proto-oncogene or by measuring activity of a gene product encoded by the proto-oncogene. The gene product may be mRNA or a polypeptide encoded by the gene.

The present invention also contemplates a method of identifying an agent that disrupts a super-enhancer associated with a proto-oncogene comprising transfecting a cell with a super-enhancer and an associated proto-oncogene under conditions suitable for the super-enhancer to drive high levels of expression of the proto-oncogene, wherein the proto-oncogene is located within an insulated neighborhood, contacting the cell with a test agent, and measuring the level of expression of the proto-oncogene, wherein decreased expression of the proto-oncogene in the presence of the test agent indicates that the test agent is an agent that disrupts the super-enhancer associated with the proto-oncogene. In some aspects, the proto-oncogene is transfected into the cell within an insulated neighborhood. In other aspects, the proto-oncogene is transfected into an insulated neighborhood already present within the cell.

Also provided is a method of identifying a screening agent that identifies a disruption in an insulated neighborhood boundary, comprising transfecting a cell with a super-enhancer and an associated proto-oncogene, wherein the proto-oncogene is located within an insulated neighborhood, contacting the cell with a screening agent, and measuring the level of expression of the screening agent, wherein increased expression of the screening agent indicates that the proto-oncogene is activated.

It should be appreciated that the various agents described herein can be used alone, or in combination with other agents, as previously discussed.

In some aspects of any screening and/or characterization methods, test agents are contacted with test cells (and optionally control cells) or used in cell-free assays at a predetermined concentration. In some embodiment the concentration is about up to 1 nM. In some embodiments the concentration is between about 1 nM and about 100 nM. In some embodiments the concentration is between about 100 nM and about 10 μM. In some embodiments the concentration is at or above 10 μM, e.g., between 10 μM and 100 μM. Following incubation for an appropriate time, optionally a predetermined time, the effect of compounds or composition on a parameter of interest in the test cells is determined by an appropriate method known to one of ordinary skill in the art, e.g., as described herein. Cells can be contacted with compounds for various periods of time. In certain embodiments cells are contacted for between 12 hours and 20 days, e.g., for between 1 and 10 days, for between 2 and 5 days, or any intervening range or particular value. Cells can be contacted transiently or continuously. If desired, the compound can be removed prior to assessing the effect on the cells.

In some aspects, the cells may be in living animal, e.g., a mammal, or may be isolated cells. Isolated cells may be primary cells, such as those recently isolated from an animal (e.g., cells that have undergone none or only a few population doublings and/or passages following isolation), or may be a cell of a cell line that is capable of prolonged proliferation in culture (e.g., for longer than 3 months) or indefinite proliferation in culture (immortalized cells). In many embodiments, a cell is a somatic cell. Somatic cells may be obtained from an individual, e.g., a human, and cultured according to standard cell culture protocols known to those of ordinary skill in the art. Cells may be obtained from surgical specimens, tissue or cell biopsies, etc. Cells may be obtained from any organ or tissue of interest. In some embodiments, cells are obtained from skin, lung, cartilage, breast, blood, blood vessel (e.g., artery or vein), fat, pancreas, liver, muscle, gastrointestinal tract, heart, bladder, kidney, urethra, prostate gland.

In some embodiments the cell is a mammalian cell. In some embodiments the cell is a human cell. In some embodiments the cell is an embryonic stem cell or embryonic stem cell-like cell. In some embodiments the cell is a muscle cell. In some embodiments the muscle cell is a myotube. In some embodiments the cell is a B cell. In some embodiments the B cell is a Pro-B cell.

In some embodiments the cell is from the brain. In some embodiments the cell is an astrocyte cell. In some embodiments the cell is from the angular gyms of the brain. In some embodiments the cell is from the anterior caudate of the brain. In some embodiments the cell is from the cingulate gyms of the brain. In some embodiments the cell is from the hippocampus of the brain. In some embodiments the cell is from the inferior temporal lobe of the brain. In some embodiments the cell is from the middle frontal lobe of the brain.

In some embodiments the cell is a naïve T cell. In some embodiments the cell is a memory T cell. In some embodiments the cell is CD4 positive. In some embodiments the cell is CD25 positive. In some embodiments the cell is CD45RA positive. In some embodiments the cell is CD45RO positive. In some embodiments the cell is IL-17 positive. In some embodiments the cell is stimulated with PMA. In some embodiments the cell is a Th cell. In some embodiments the cell is a Th17 cell. In some embodiments the cell is CD255 positive. In some embodiments the cell is CD127 positive. In some embodiments the cell is CD8 positive. In some embodiments the cell is CD34 positive.

In some embodiments the cell is from the duodenum. In some embodiments the cell is from smooth muscle tissue of the duodenum.

In some embodiments the cell is from skeletal muscle tissue. In some embodiments the cell is a myoblast cell. In some embodiments the cell is a myotube cell.

In some embodiments the cell is from the stomach. In some embodiments the cell is from smooth muscle tissue of the stomach.

In some embodiments the cell is CD3 positive. In some embodiments the cell is CD8 positive. In some embodiments the cell is CD14 positive. In some embodiments the cell is CD19 positive. In some embodiments the cell is CD20 positive. In some embodiments the cell is CD34 positive. In some embodiments the cell is CD56 positive.

In some embodiments the cell is from the colon. In some embodiments the cell is a crypt cell. In some embodiments the cell is a colon crypt cell.

In some embodiments the cell is from the intestine. In some embodiments the cell is from the large intestine. In some embodiments the intestine is from a fetus.

In some embodiments the cell is a DND41 cell. In some embodiments the cell is a GM12878 cell. In some embodiments the cell is a H1 cell. In some embodiments the cell is a H2171 cell. In some embodiments the cell is a HCC1954 cell. In some embodiments the cell is a HCT-116 cell. In some embodiments the cell is a HeLa cell. In some embodiments the cell is a HepG2 cell. In some embodiments the cell is a HMEC cell. In some embodiments the cell is a HSMM tube cell. In some embodiments the cell is a HUVEC cell. In some embodiments the cell is a IMR90 cell. In some embodiments the cell is a Jurkat cell. In some embodiments the cell is a K562 cell. In some embodiments the cell is a LNCaP cell. In some embodiments the cell is a MCF-7 cell. In some embodiments the cell is a MM1S cell. In some embodiments the cell is a NHLF cell. In some embodiments the cell is a NHDF-Ad cell. In some embodiments the cell is a RPMI-8402 cell. In some embodiments the cell is a U87 cell.

In some embodiments the cell is an osteoblast cell. In some embodiments the cell is from the pancreas. In some embodiments the cell is from a pancreatic cancer cell.

In some embodiments the cell is from adipose tissue. In some embodiments the cell is from the adrenal gland. In some embodiments the cell is from the bladder. In some embodiments the cell is from the esophagus. In some embodiments the cell is from the stomach. In some embodiments the cell is a gastric cell. In some embodiments the cell is from the left ventricle. In some embodiments the cell is from the lung. In some embodiments the cell is from a lung cancer cell. In some embodiments the cell is a fibroblast cell.

In some embodiments the cell is from the ovary. In some embodiments the cell is from the psoas muscle. In some embodiments the cell is from the right atrium. In some embodiments the cell is from the right ventricle. In some embodiments the cell is from the sigmoid colon. In some embodiments the cell is from the small intestine. In some embodiments the cell is from the spleen. In some embodiments the cell is from the thymus.

In some embodiments the cell is a VACO 9M cell. In some embodiments the cell is a VACO 400 cell. In some embodiments the cell is a VACO 503 cell.

In some embodiments the cell is from the aorta.

In some embodiments the cell is from the brain. In some embodiments the cell is a brain cancer cell.

In some embodiments the cell is from the breast. In some embodiments the cell is a breast cancer cell.

In some embodiments the cell is from the cervix. In some embodiments the cell is a cervical cancer cell.

In some embodiments the cell is from the colon. In some embodiments the cell is from a colorectal cancer cell.

In some embodiments the cell is a blood cell. In some embodiments the blood cell is a monocyte cell. In some embodiments the blood cell is a B cell. In some embodiments the blood cell is a T cell. In some embodiments the blood cell is a human embryonic stem cell. In some embodiments the blood cell is a cancerous blood cell. In some embodiments the blood cell is from a fetus.

In some embodiments the cell is from bone. In some embodiments the bone cell is an osteoblast cell.

In some embodiments the cell is from the heart. In some embodiments the cell is a mammary epithelial cell. In some embodiments the cell is a skin cell. In some embodiments the skin cell is a fibroblast cell.

In some embodiments the cell is an embryonic stem cell. In some embodiments the cell is from the umbilical vein. In some embodiments the cell from the umbilical vein is an endothelial cell.

In some embodiments the cell is from the colon. In some embodiments the cell is from the prostate. In some embodiments the cell is a prostate cancer cell.

In some embodiments the cell is from the liver. In some embodiments the cell is a liver cancer cell.

In some embodiments the cell is from the muscle. In some embodiments the muscle is from a fetus.

In some embodiments the cell is from the thymus. In some embodiments the thymus is from a fetus.

Cells may be maintained in cell culture following their isolation. In certain embodiments, the cells are passaged or allowed to double once or more following their isolation from the individual (e.g., between 2-5, 5-10, 10-20, 20-50, 50-100 times, or more) prior to their use in a method of the invention. They may be frozen and subsequently thawed prior to use. In some embodiments, the cells will have been passaged or permitted to double no more than 1, 2, 5, 10, 20, or 50 times following their isolation from the individual prior to their use in a method of the invention. Cells may be genetically modified or not genetically modified in various embodiments of the invention. Cells may be obtained from normal or diseased tissue. In some embodiments, cells are obtained from a donor, and their state or type is modified ex vivo using a method of the invention. The modified cells are administered to a recipient, e.g., for cell therapy purposes. In some embodiments, the cells are obtained from the individual to whom they are subsequently administered.

A population of isolated cells in any embodiment of the invention may be composed mainly or essentially entirely of a particular cell type or of cells in a particular state. In some embodiments, an isolated population of cells consists of at least 30%, 40%, 50%, 60%, 70%, 80%, 90%, 95%, 96%, 97%, 98%, 99%, or 100% cells of a particular type or state (i.e., the population is at least 30%, 40%, 50%, 60%, 70%, 80%, 90%, 95%, 96%, 97%, 98%, 99%, or 100% pure), e.g., as determined by expression of one or more markers or any other suitable method.

It is to be understood that the inventions disclosed herein are not limited in their application to the details set forth in the description or as exemplified. The invention encompasses other embodiments and is capable of being practiced or carried out in various ways. Also, it is to be understood that the phraseology and terminology employed herein is for the purpose of description and should not be regarded as limiting.

While certain compositions, methods and assays of the present invention have been described with specificity in accordance with certain embodiments, the following examples serve only to illustrate the methods and compositions of the invention and are not intended to limit the same. The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications and other reference materials referenced herein to describe the background of the invention and to provide additional detail regarding its practice are hereby incorporated by reference.

### EXAMPLES

### Example 1: 3D Regulatory Landscape of Human Naive and Primed Embryonic Stem Cells (ESCs)

With the recent isolation of naive human embryonic stem cells (Chan et al., 2013;

Gafni et al., 2013; Takashima et al., 2014; Theunissen et al., 2014; Ware et al., 2014), it is possible to deduce the 3D regulatory landscape of one of the earliest stages of human development. Naive ESCs represent the ground state of pluripotency and are capable of forming all differentiated cell types (De Los Angeles et al., 2012; Hackett and Surani, 2014; Martello and Smith, 2014). Primed ESCs are pluripotent, but represent a subsequent post-implantation epiblast cell state that has a developmental bias towards the ectoderm (De Los Angeles et al., 2012; Hackett and Surani, 2014; Martello and Smith, 2014). Defining the 3D regulatory landscape of these two cell states should prove to be valuable for understanding the transcriptional control of early human development.

To deduce the 3D regulatory landscape of naive and primed human embryonic stem cells, the present inventors identified and describe herein enhancers, insulators and cohesin-associated chromatin interactions. The results presented herein indicate that cohesin-associated loops are largely responsible for the organization of TADs: cohesin-associated CTCF-CTCF loops span a large fraction of TADs and form internal structures within TADs. Enhancers interact with specific genes located within the CTCF-CTCF loops, indicating that they function as insulated neighborhoods. The CTCF sites that contribute to these loops are highly conserved, and loss of these sites occurs frequently in cancer. The present inventors thus provide an initial map of the 3D regulatory landscapes of human pluripotent cells and a foundation for further studies of the relationships between chromosome structure and gene control in development and disease.

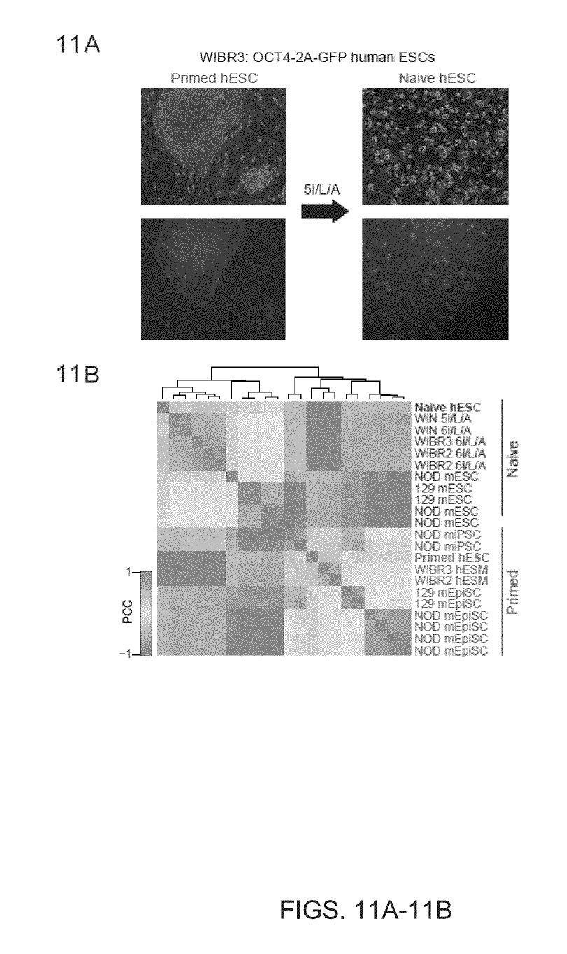

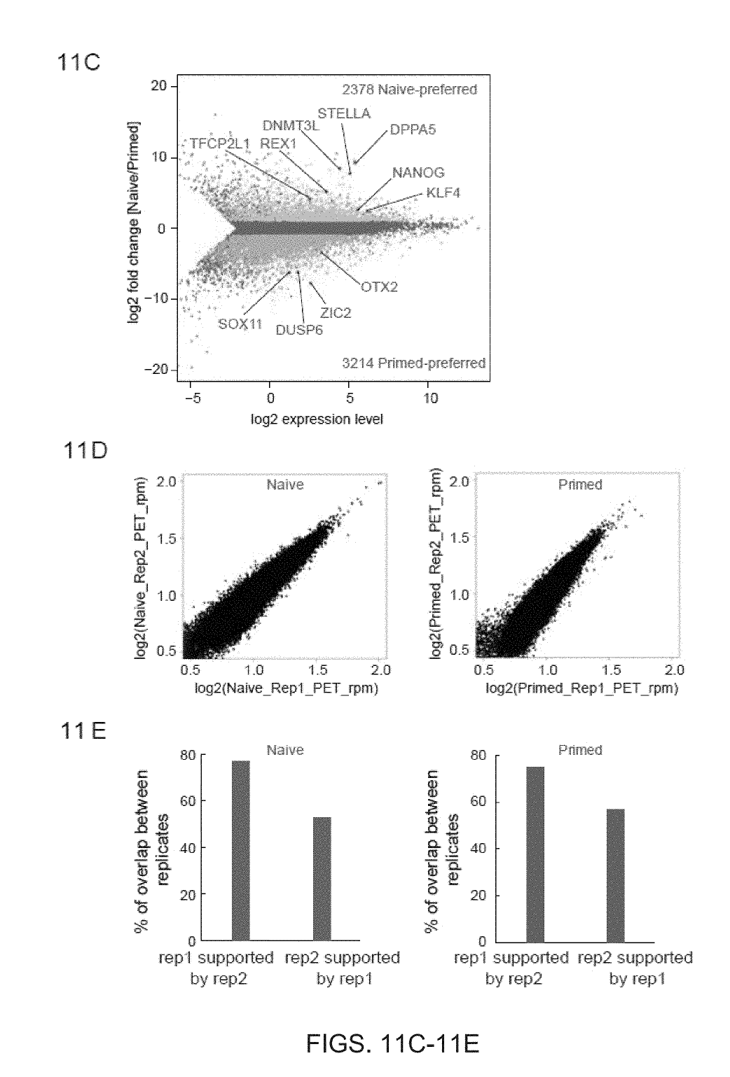

To investigate the 3D regulatory landscape of naive hESCs and the isogenic primed hESCs from which the naive cells were derived, the present inventors generated populations of both cell states and reinvestigated their morphology and gene expression programs to confirm that they maintained key features previously described for these cells reproducibly (Theunissen et al., 2014). As expected, the colonies of naive hESCs exhibited a dome-shaped morphology and the colonies of primed hESCs had a flat morphology (FIG. 11A). The gene expression programs were reinvestigated by generating high-quality RNA-seq datasets (Extended Experimental Procedures). Cross-species clustering confirmed that the naive and primed hESC gene expression datasets were highly similar to those previously established for naive and primed hESCs as well as their murine counterparts (FIG. 11B) (Theunissen et al., 2014). Further analysis of the RNA-seq data confirmed that genes previously noted as preferentially expressed in either naive or primed hESCs were indeed preferentially expressed in these RNA-seq datasets (FIG. 11C) (Takashima et al., 2014; Theunissen et al., 2014). A complete list of genes that are preferentially expressed in the naive or primed hESCs can be found in Table S12. These results confirm that the conditions used for growth and maintenance of these isogenic naive and primed human pluripotent states are reproducible (Theunissen et al., 2014).

### Enhancers, Insulators and Cohesin-Associated DNA Interactions in Human ESCs

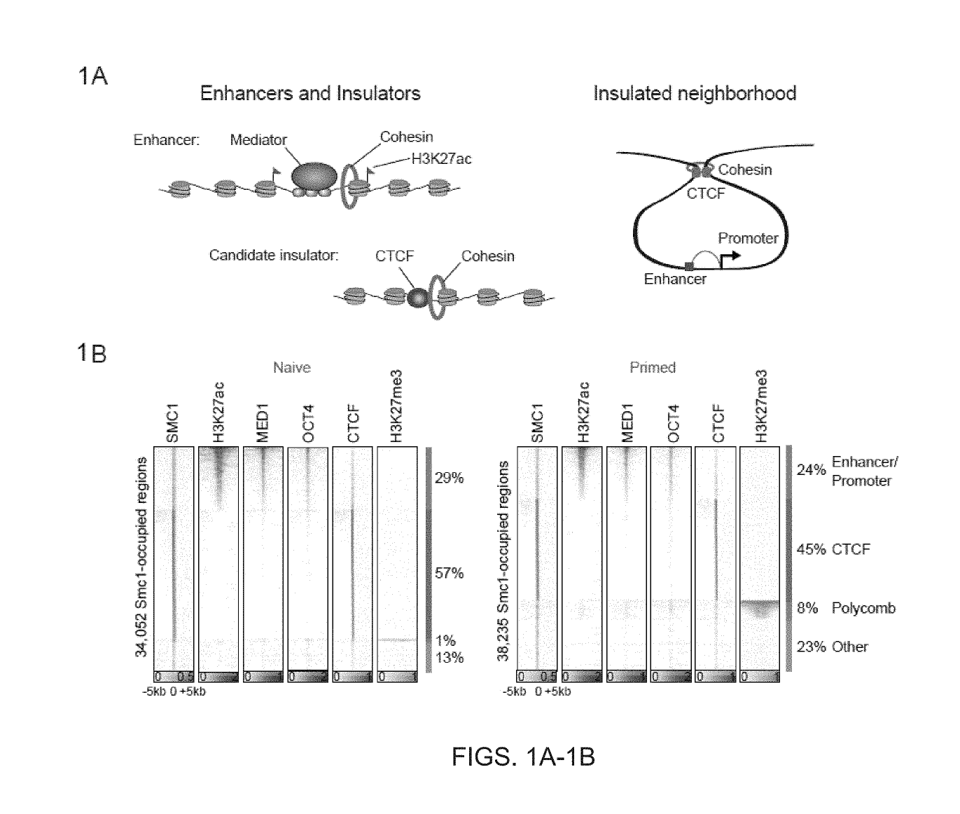

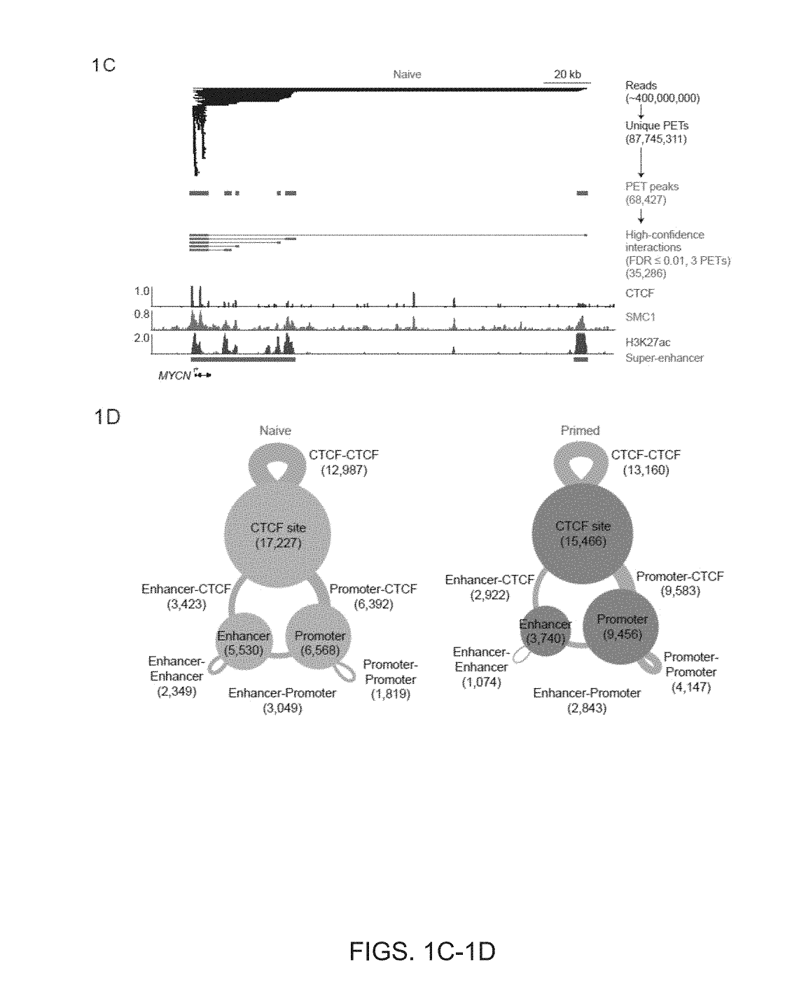

Enhancers and insulators are cis-regulatory elements that can be identified by the regulatory proteins that occupy them and by the looped structures that are formed by cohesin-associated interactions (FIG. 1A). For both naive and primed ESCs, the present inventors identified regions occupied by cohesin and then identified putative enhancers and insulators (FIG. 1B, Table S13). Enhancers were identified by generating ChIP-seq data for histone H3K27ac and confirmed with ChIP-seq data for the MED1 subunit of Mediator and the OCT4 master transcription factor (FIG. 1B, Table S14). Candidate insulators were identified by determining the genome-wide occupancy of CTCF (FIG. 1B, Table S15). The data for the naive ESCs indicates that ˜29% of cohesin-occupied sites involve active enhancers and promoters and ˜57% involve CTCF sites that are not associated with enhancers and promoters (FIG. 1B). Similar results were obtained for the primed ESCs, except there was a substantial fraction of cohesin-occupied regions that were associated with polycomb modifications (FIG. 1B), as noted previously (Theunissen et al., 2014).

To identify cohesin-associated loops, the present inventors generated ChIA-PET data for cohesin in both naive and primed hESCs. The ChIA-PET technique was used because it yields genome-wide, high-resolution (˜4 kb) interaction data coupled to the location of a specific protein, thus providing mechanistic insight into that set of chromatin interactions (DeMare et al., 2013; Dowen et al., 2014; Fullwood et al., 2009; Handoko et al., 2011; Heidari N, 2014; Kieffer-Kwon et al., 2013; Li et al., 2012; Maejima et al., 2014). Cohesin for ChIA-PET was selected because it is a relatively well-studied SMC complex that is loaded at enhancer-promoter loops and can thus identify those interactions, and can also occupy CTCF sites and thus identify those interactions as well (Dowen et al., 2013; Dowen et al., 2014; Hadjur et al., 2009; Kagey et al., 2010; Nativio et al., 2009; Parelho et al., 2008; Rubio et al., 2008; Schmidt et al., 2012; Wendt et al., 2008).

Biological replicate ChIA-PET datasets for the cohesin subunit SMC1 were generated for both the naive and primed hESCs. A total of ˜400 million reads were acquired for both naive and primed hESCs (Table S16, Table S17). The respective replicates showed a high degree of correlation (Pearson's r >0.98, FIGS. 11D-11E), so replicate data was pooled and processed as previously described (Dowen et al., 2014), with modifications described in the Extended Experimental Procedures. The naive hESC dataset contained ˜88 million unique paired-end tags (PETs) that identified 35,286 high confidence cohesin-associated intra-chromosomal interactions (Table S16), and the primed hESC dataset contained ˜125 million unique PETs that identified 46,257 high confidence cohesin-associated intra-chromosomal interactions (Table S17). The results for the MYCN locus in naive hESCs and the effects of filtering for high-confidence interactions are shown in FIG. 1C. At this locus, multiple interactions between super-enhancer constituents and the MYCN promoter are observed. A summary of the high confidence interactions identified in naive and primed hESCs is shown in FIG. 1D. These high confidence interactions were used for further analyses unless otherwise stated.

### Cohesin-Associated Loops Organize TADS

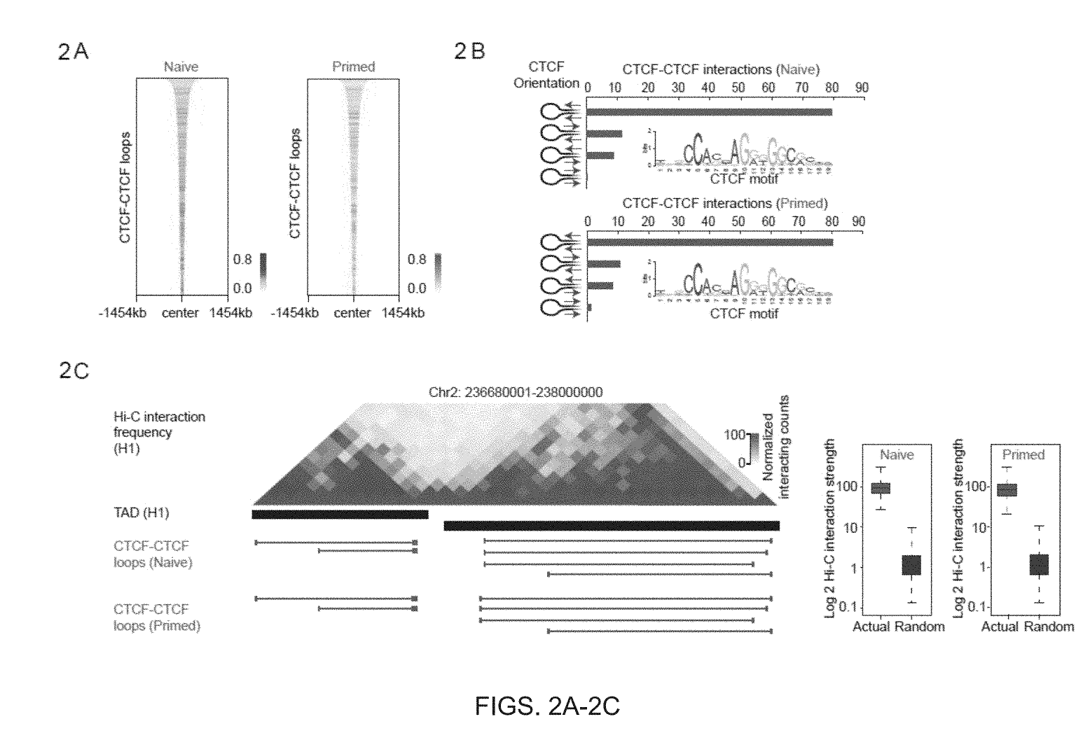

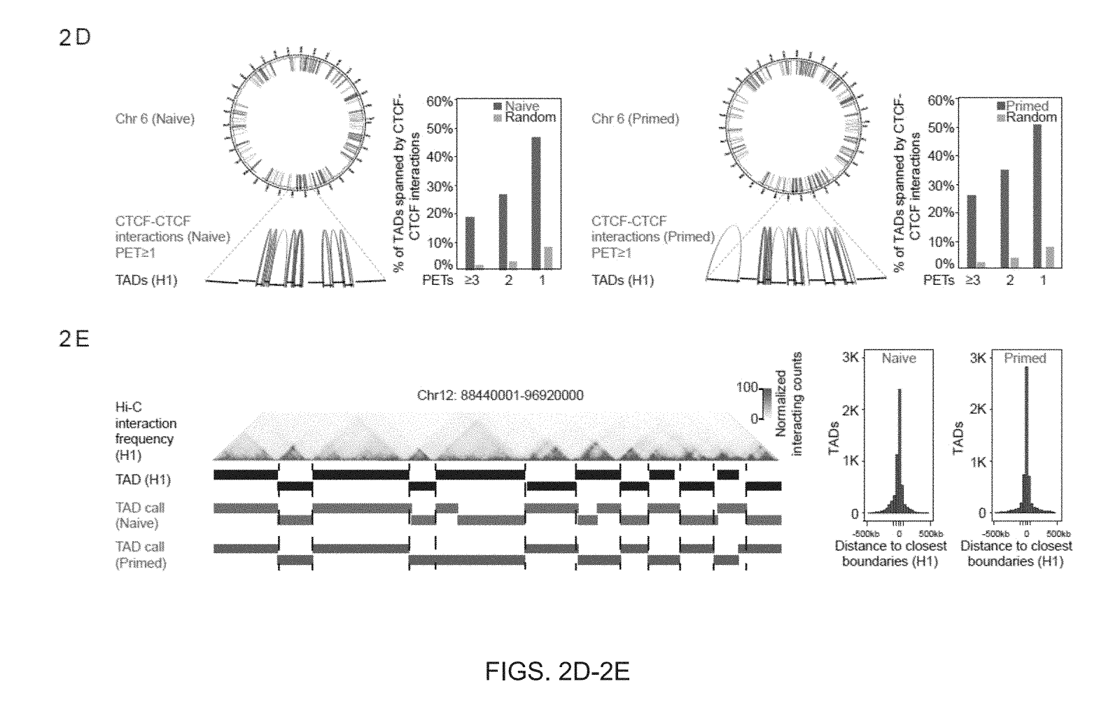

The present investigators first studied the cohesin-associated DNA loops that occur between two CTCF-bound sites in the two hESCs and found that the majority (80%) of such loops in naive hESCs were also found in the primed hESCs (FIG. 2A). There were 12,987 CTCF-CTCF loops in naive hESCs, encompassing 37% of the genome and 33% of protein-coding genes (Table S16). These CTCF-CTCF loops ranged from 4 kb to >800 kb and contained 0-24 protein-coding genes, with a median of 200 kb and 1 protein-coding gene per loop. Similar numbers were obtained for the primed human ESCs (Table S17). Previous studies have noted that when CTCF homo-dimers form DNA loops, the two CTCF sequence motifs that are bound occur in specific orientations (Filippova et al., 1996; Rao et al., 2014), and the present investigators found that the two occupied sites that contribute to CTCF loops do occur predominantly in the convergent orientation (FIG. 2B).

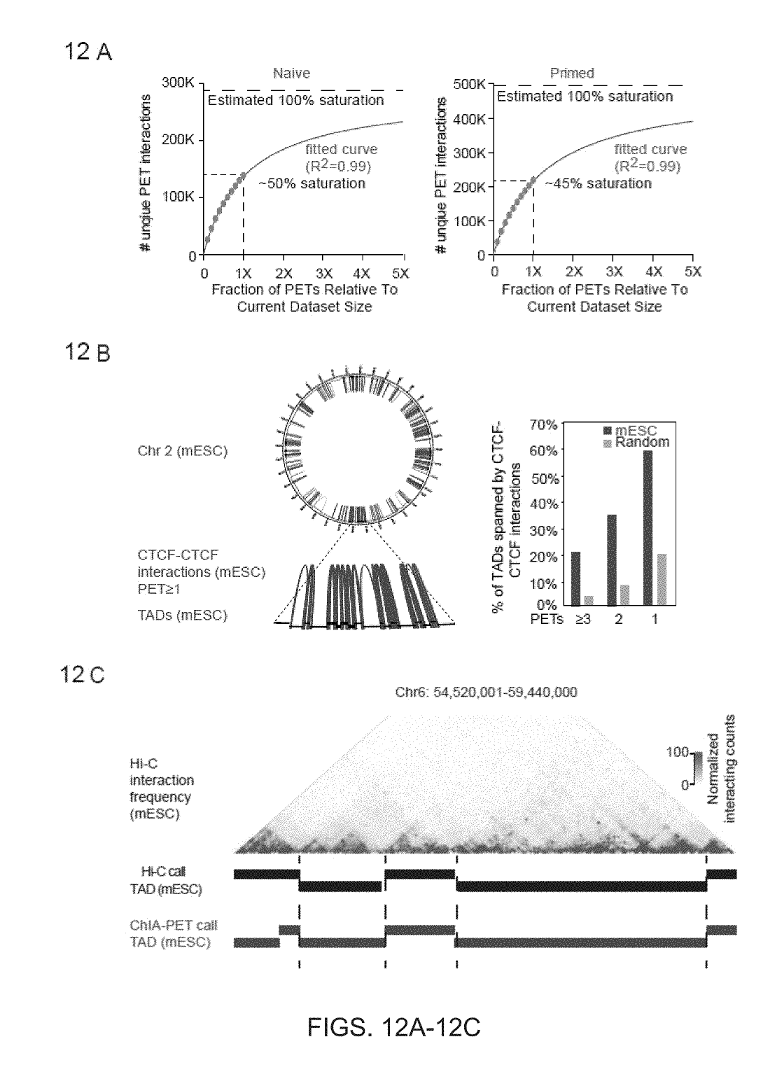

Recent studies have noted a degree of correspondence between CTCF-CTCF loops and TAD structures (Dowen et al., 2014; Rao et al., 2014). A comparison of the CTCF-CTCF loops identified here with TADs identified previously in H1 ESCs (primed hESCs) with Hi-C data (Dixon et al., 2015) revealed several especially striking features. The CTCF-CTCF loops almost always occurred within TADs and showed interactions that closely corresponded to the Hi-C interaction heatmap (FIG. 2C). Genome-wide analysis indicated that the CTCF-CTCF loops correlate with the H1 hESC Hi-C signal to a striking degree and much more than would be expected at random (FIG. 2C). We found evidence for CTCF-CTCF loops that spanned entire TADs for a large fraction of TADs (FIG. 2D). The boundaries of four TADs have been shown by FISH to be in very close physical proximity in human lymphoblastoid cells (Rao et al., 2014), and all four have TAD-spanning CTCF-CTCF loops in these hESCs. Saturation analysis indicated that the ChIA-PET datasets are approximately 50% complete (FIG. 12A), indicating that only a subset of all CTCF-CTCF loops were identified in the high confidence data, so it is possible that most TAD boundaries are defined by CTCF-CTCF loops. These results suggest that CTCF-CTCF loops make major contributions to TAD structure.

If cohesin-associated loops play a major role in TAD structure, it should be possible to reconstruct TADs, which were previously derived solely from Hi-C data, by using cohesin ChIA-PET data. The results shown in FIG. 2E confirm that the cohesin ChIA-PET data, processed using the same Hidden Markov algorithm used to process Hi-C data, can capture most TAD boundaries derived from Hi-C data in H1 hESCs. This observation led us to determine whether similar results could be obtained for murine ESC TADs using previously generated murine ESC cohesin ChIA-PET data; the results confirmed that CTCF-CTCF loops span a substantial portion of TADS (FIG. 12B) and that cohesin ChIA-PET data and Hi-C data produce similar TAD structures (FIG. 12C). These results are consistent with the idea that cohesin-associated DNA interactions provide much of the underlying structure of TADs.

### CTCF-CTCF Loops Form Insulated Neighborhoods

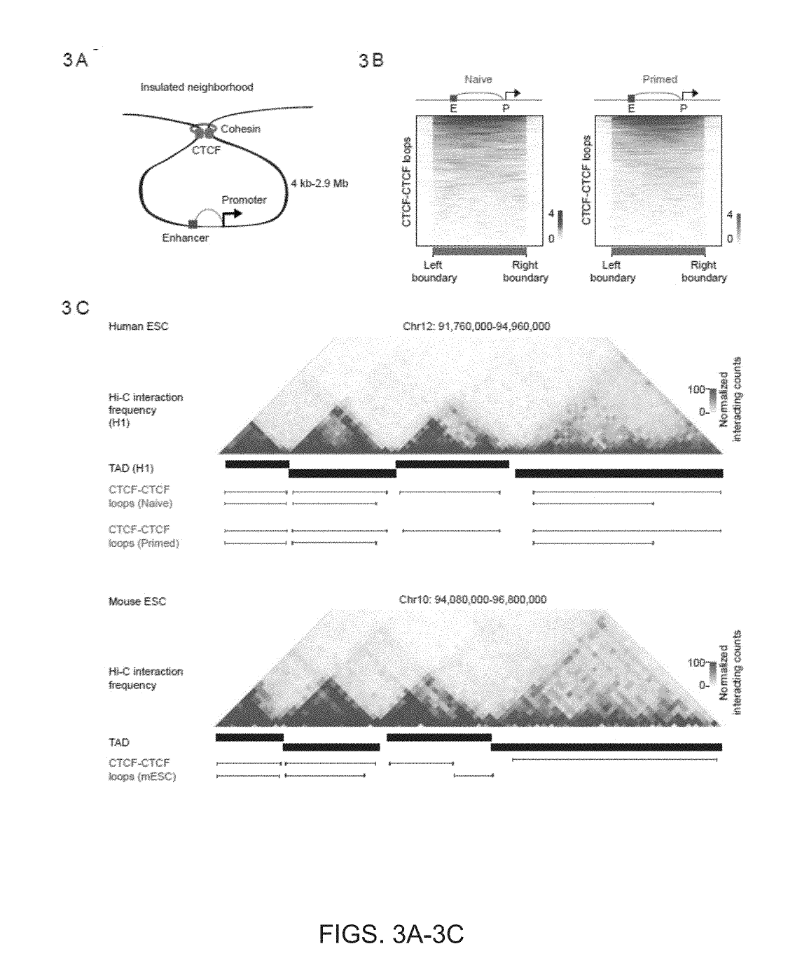

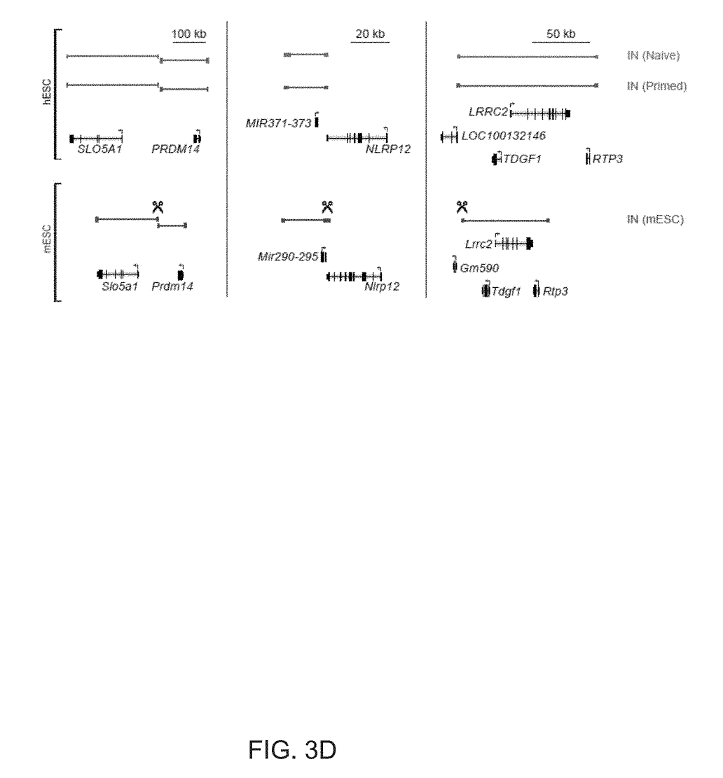

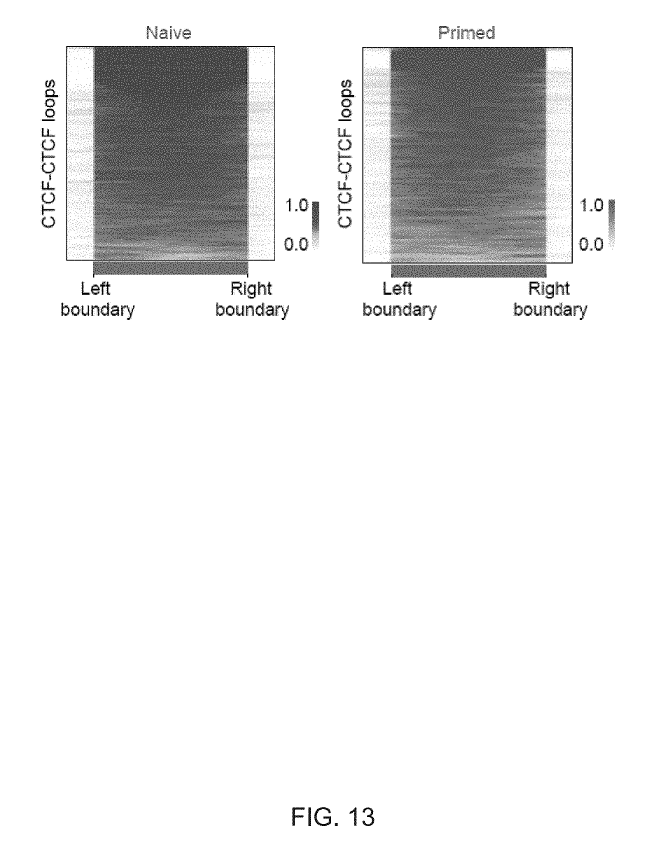

Previous studies in murine ESCs showed that CTCF-CTCF loops containing active pluripotency genes or silent polycomb-associated genes can function as insulated neighborhoods for gene control (FIG. 3A) because DNA interactions between regulatory elements and genes occur within the CTCF-CTCF loops and the loss of either of the CTCF sites alters gene regulation within and immediately outside the loop (Dowen et al., 2014). If the CTCF-CTCF loops identified in hESC function as insulated neighborhoods, we expect that most cohesin-associated interactions with an endpoint inside the loop have their other endpoint within the loop. The results in FIG. 13 confirm that interactions that originate within a CTCF-CTCF loop almost invariably end within the boundaries of the loop in naive and primed hESCs. Furthermore, the present inventors found that enhancers generally interact with a target gene within the CTCF-CTCF loops (FIG. 3B), consistent with the view that the CTCF-CTCF loops constrain enhancer-promoter interactions within the loops.

We examined CTCF-CTCF loops in syntenic regions of human and mouse chromosomes to ascertain whether they are conserved, as has been observed previously for TADs (Dixon et al., 2012). Examination of the CTCF-CTCF loop structures in murine and human ESCs revealed that they are largely preserved in these syntenic regions (FIG. 3C). We found that the CTCF boundary elements that were shown to function as insulated neighborhood boundaries in murine ESCs (Dowen et al., 2014) have counterparts in the hESCs studied here, and these CTCF-CTCF loops contain human homologues of the murine pluripotency genes (FIG. 3D). Thus, cohesin-associated CTCF-CTCF loops are largely preserved between syntenic regions of human and mouse, where conserved boundary CTCF sites have previously been shown to be essential for insulator function in the mouse.

### Models of TAD Structures

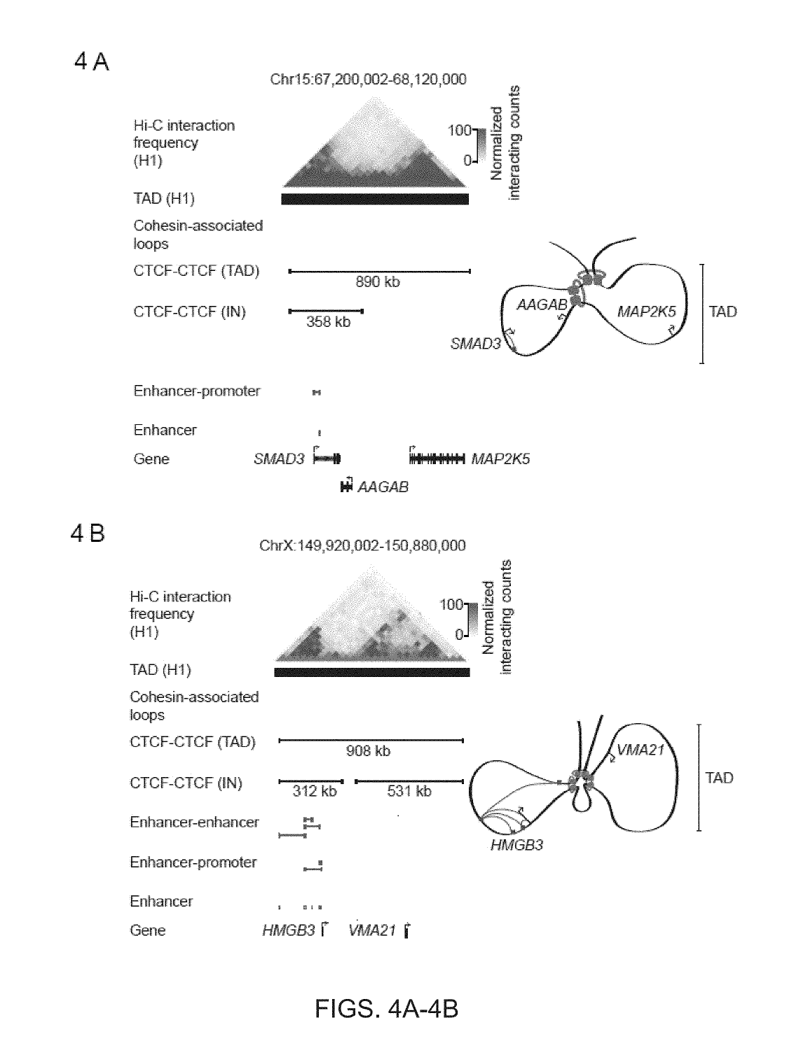

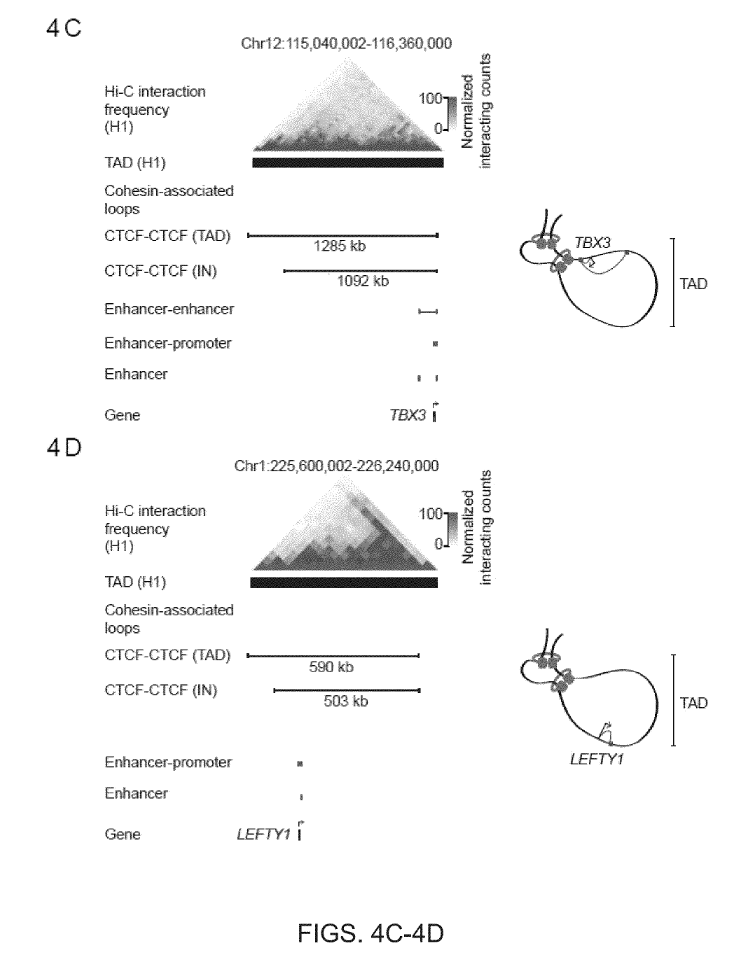

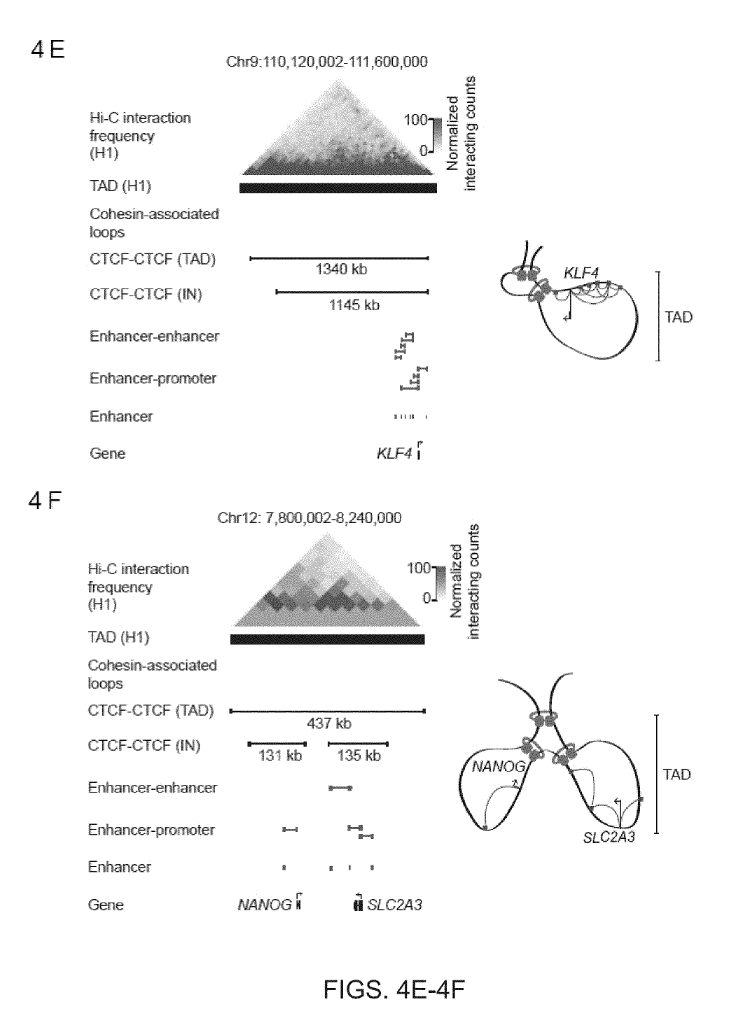

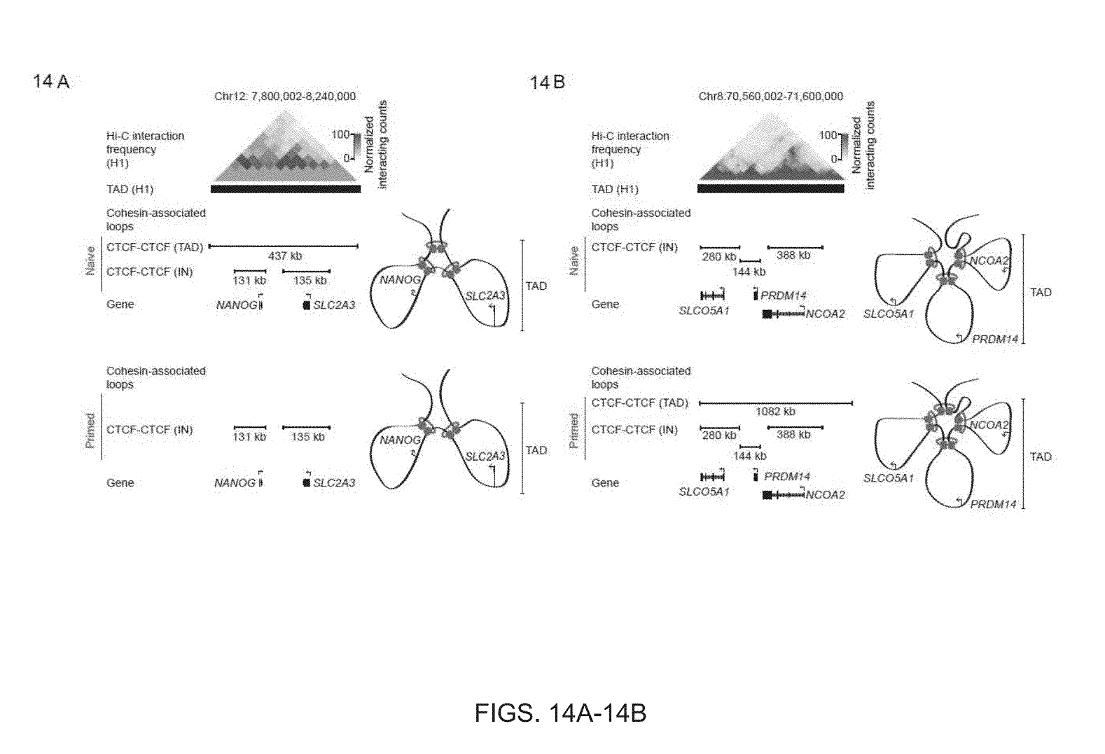

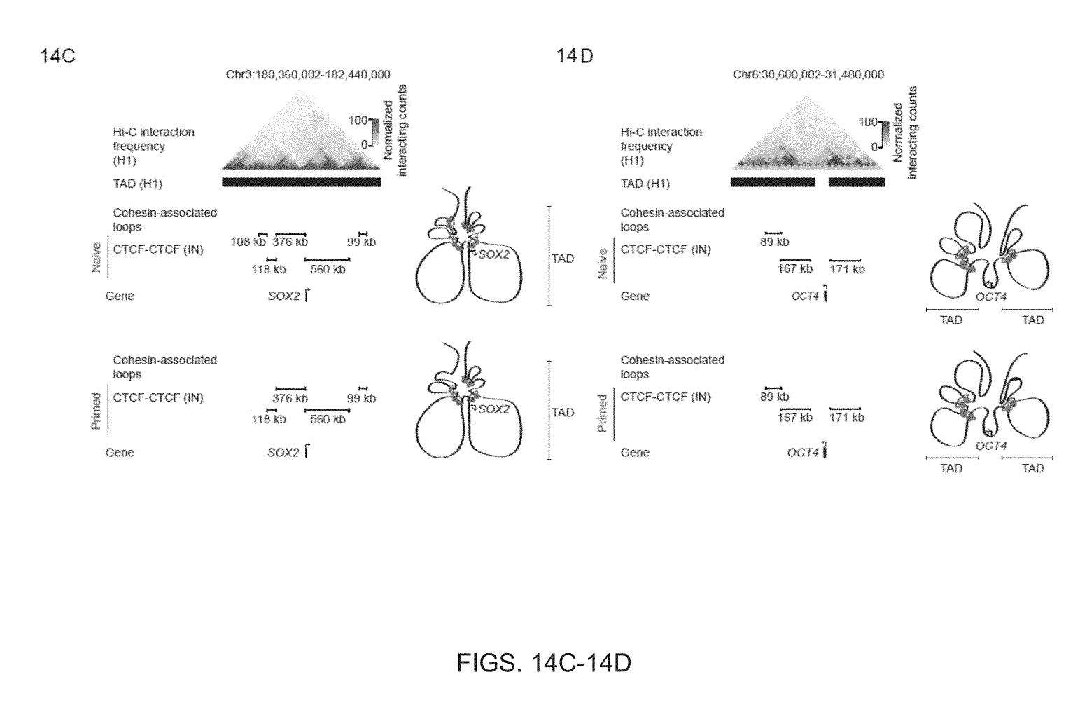

We assembled structural models of TADs based on CTCF-CTCF loops and enhancer-promoter loops and show a subset that illustrate common themes (FIG. 4; FIG. 14). CTCF-CTCF loops frequently span TADs, effectively forming one large insulated neighborhood. In some TADs, nested CTCF-CTCF interactions occur such that genes are embedded within two or more independent CTCF-CTCF loops. Cohesin-associated enhancer-promoter interactions essentially always occur within the smallest CTCF-CTCF loop formed within the TAD where the gene occurs, again consistent with the idea that the CTCF-CTCF loops have insulating properties. The CTCF-CTCF loop structures of TADs in naive and primed cells were very similar, although there were some instances where a TAD-spanning loop or an internal loop identified in one of the pluripotent cells was absent in data for the other cell (FIG. 14).

The TAD models assembled with cohesin-associated loop data likely represent the minimal set of scaffold structures, as the cohesin ChIA-PET data is not saturating and low-confidence data is not included. Nonetheless, it is useful to use this to estimate the minimal frequencies of TAD substructures due to CTCF-CTCF loops. TADs range in size from 0.2-21 Mb and contain 0-768 genes. The median number of CTCF-CTCF insulated neighborhoods that occurred within each TAD was 2 and these ranged from 4 kb to 2.9 Mb and contained 0-24 protein-coding genes. In this V1.0 map of TAD-containing insulated neighborhoods, the median neighborhood was 200 kb and contained one gene whose average size was approximately 30 kb.

Differential gene control in naive and primed hESCs

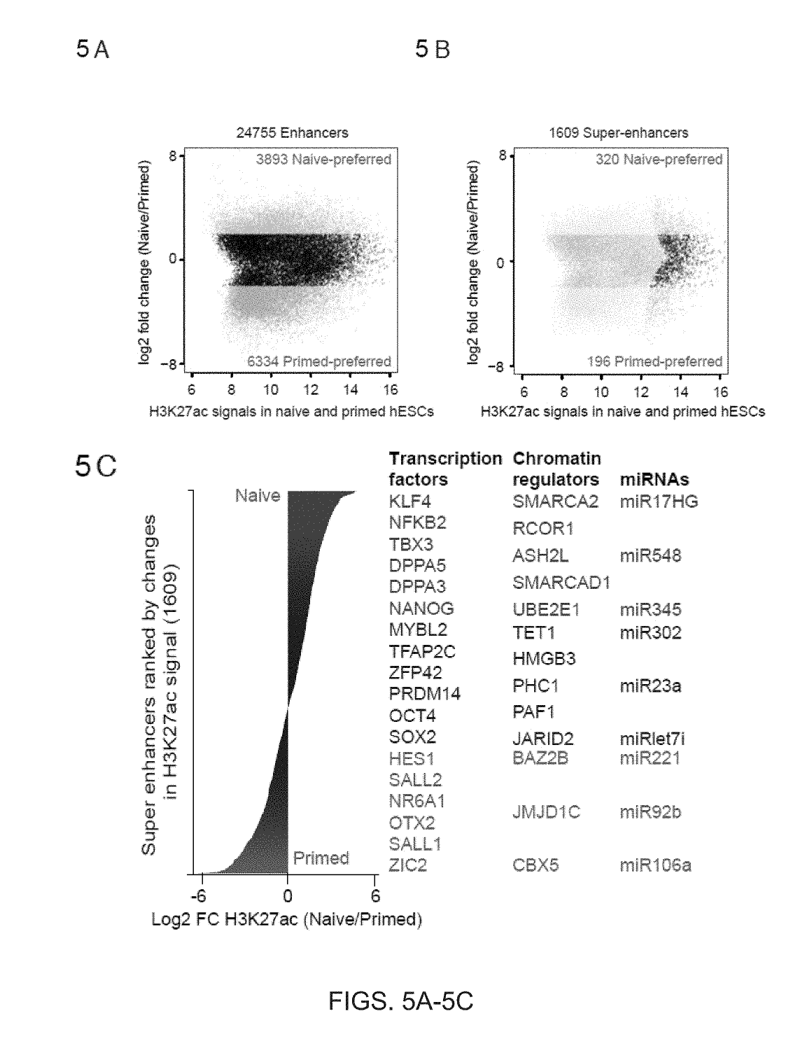

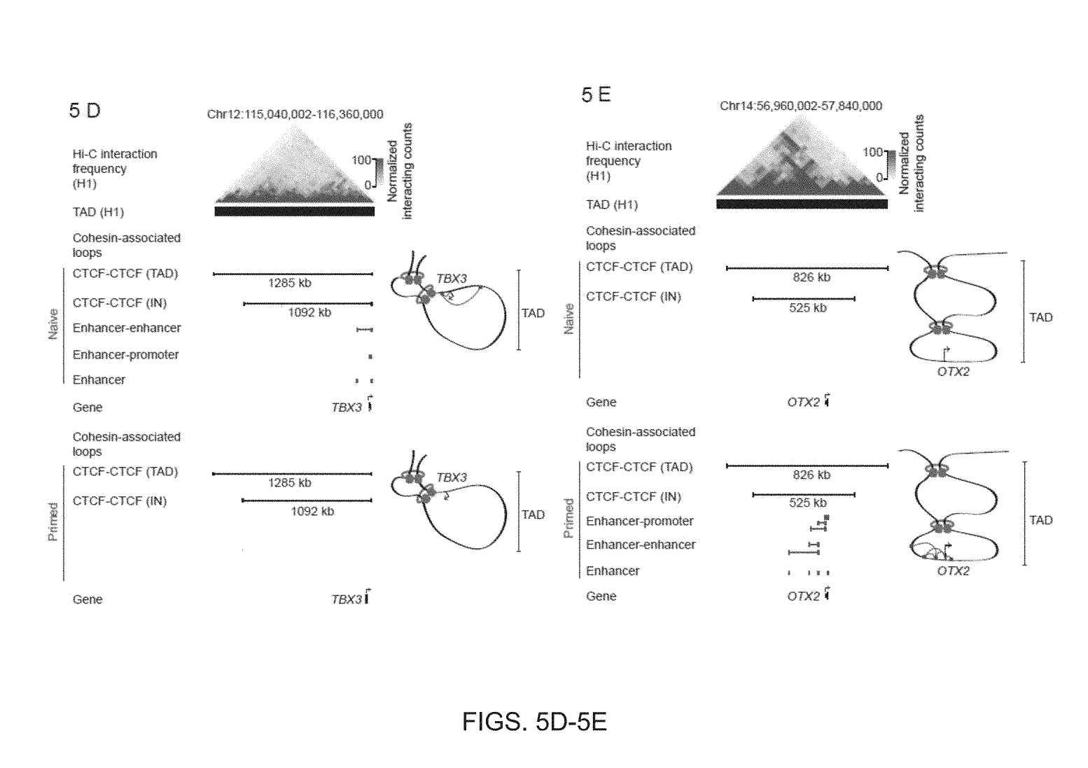

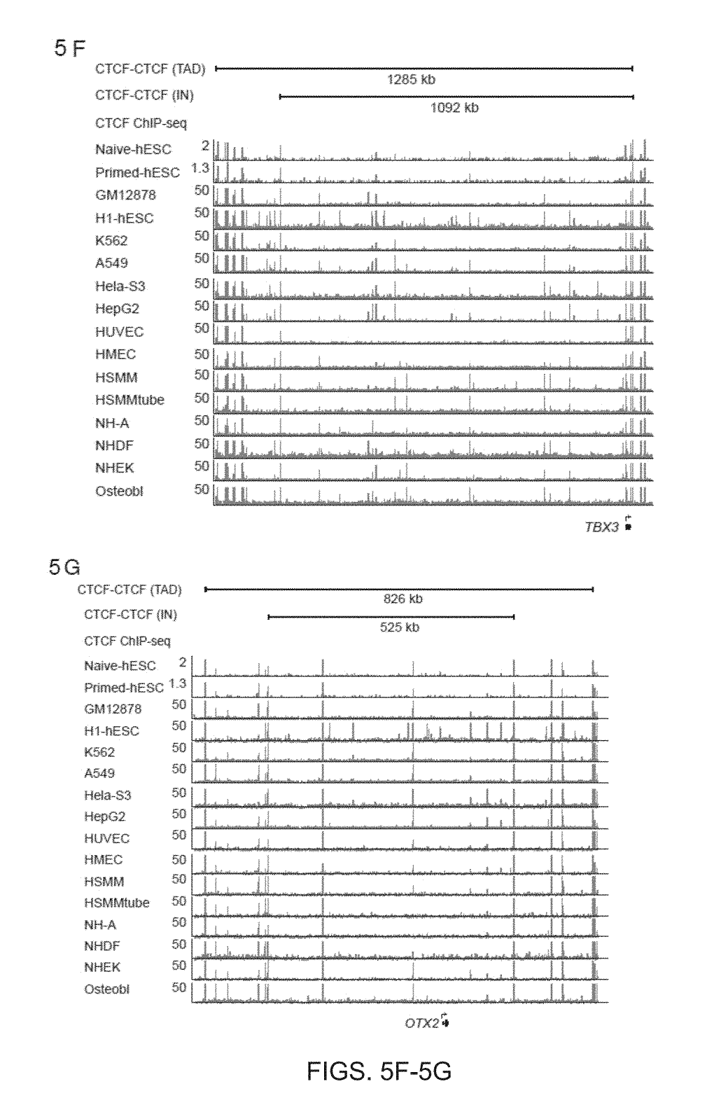

The expression programs of naive and primed ESCs are highly similar, but there are genes that are differentially expressed and thus distinguish each cell type (FIG. 11C). To gain insights into the differential regulation of genes that contribute to these two states of pluripotency, we compared the enhancer landscapes of the two cell types (FIG. 5A). Of the 24,755 active enhancers identified in naive and primed hESCs using H3K27ac ChIP-seq data, 16% showed >2-fold H3K27ac signal in naive hESCs relative to primed hESCs and 26% showed >2-fold H3K27ac signal in primed hESCs relative to naive hESCs (FIG. 5A). To focus on genes likely to contribute to the control of these pluripotent states, we concentrated our analysis on super-enhancers and their associated genes (FIGS. 5B-5C; Table S18) because super-enhancers are known to drive expression of key pluripotency genes in mESCs (Whyte et al., 2013). The super-enhancer-associated genes encode many transcription factors, chromatin regulators and miRNAs that have previously been implicated in the control of pluripotency (Bilodeau et al., 2009; Chia et al., 2010; Ding et al., 2009; Fazzio et al., 2008; Hu et al., 2009; Kagey et al., 2010).

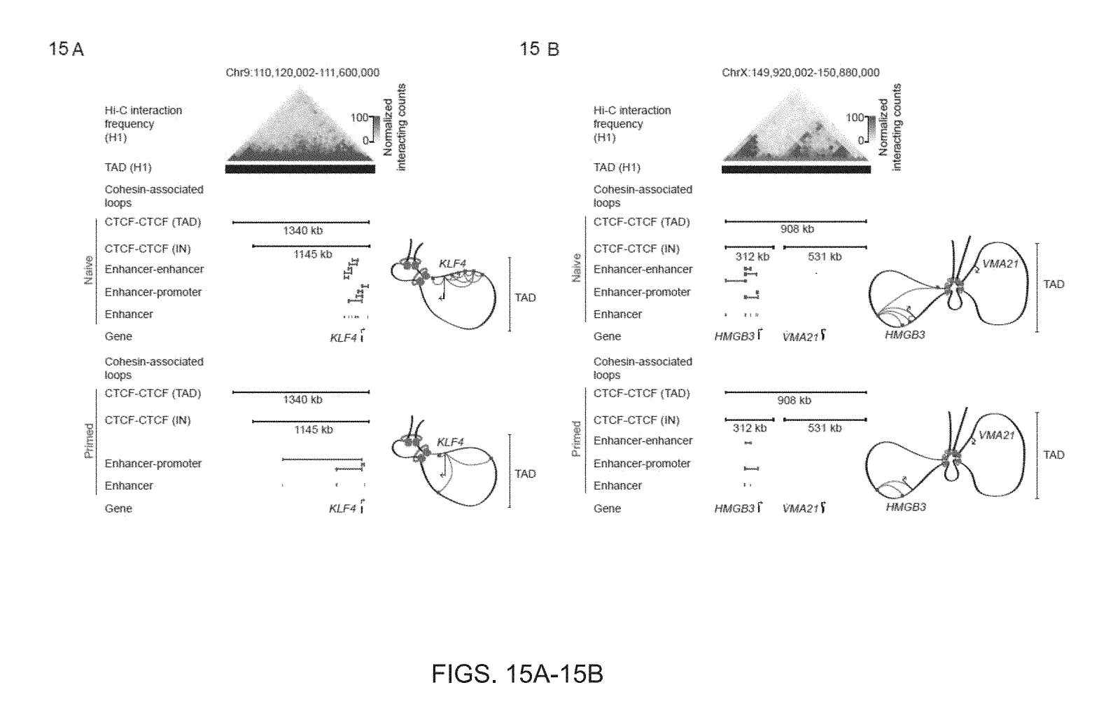

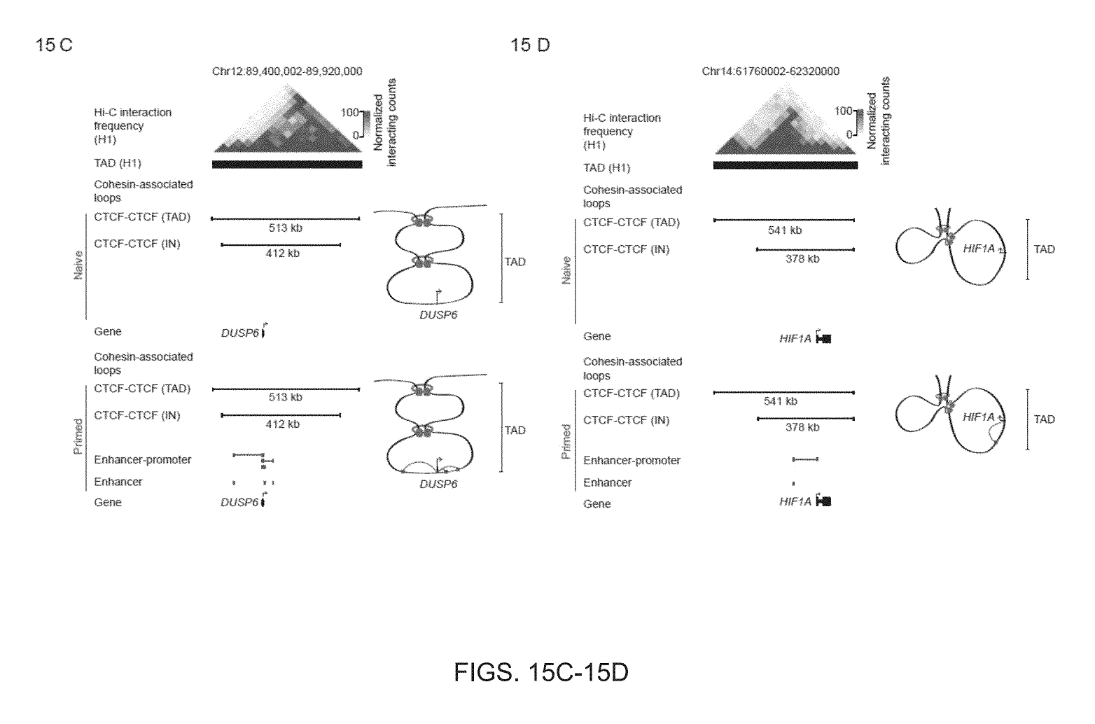

Differentially expressed pluripotency genes tended to occur in similar TAD CTCF-CTCF loops structures in naive and primed cells, but showed evidence for differential enhancer activity (FIGS. 5D-5E; FIG. 15). Inspection of 3D chromosome structure at loci for genes that have naive-preferred enhancers and are preferentially expressed in naive hESCs revealed that they share cohesin-associated CTCF-CTCF structures in naive and primed hESCs, as shown for TBX3 in FIG. 5D. TBX3 has a super-enhancer only in naive cells and is expressed 25-fold higher in naive than primed cells. Similarly, many genes that are preferentially expressed in primed hESCs occur within shared CTCF-CTCF structures, as shown for OTX2 in FIG. 5E. OTX2 has a super-enhancer only in primed cells and is expressed 10-fold higher in primed cells than in naive cells. The theme of differential expression within the context of similar CTCF-CTCF loops was observed in many additional TADs (FIG. 15). These results indicate that differential expression of a key set of pluripotency genes occurs in the context of conserved structural scaffolds composed of CTCF-CTCF loops in naive and primed hESCs.

The CTCF sites at CTCF-CTCF loop anchors in the hESCs are consistently bound by CTCF in many other human cell types, as exemplified by ChIP-seq data for 16 different cell types at the TBX3 and OTX2 loci (FIGS. 5F-5G), so these may contribute to similar loop structures in differentiated cells. Similar evidence for consistent binding of CTCF in multiple cell types has been described (Chen et al., 2012; Cuddapah et al., 2009; Dowen et al., 2014;

Heidari et al., 2014; Kim et al., 2007; Phillips-Cremins et al., 2013; Schmidt et al., 2012; Wang et al., 2012). This reinforces the idea that CTCF, together with cohesin, generates similar chromosomal scaffolds in different cells and that transcriptional regulatory elements function within this context to produce cell-type specific gene expression programs.

Conservation of 3D Structure and Associations with Disease

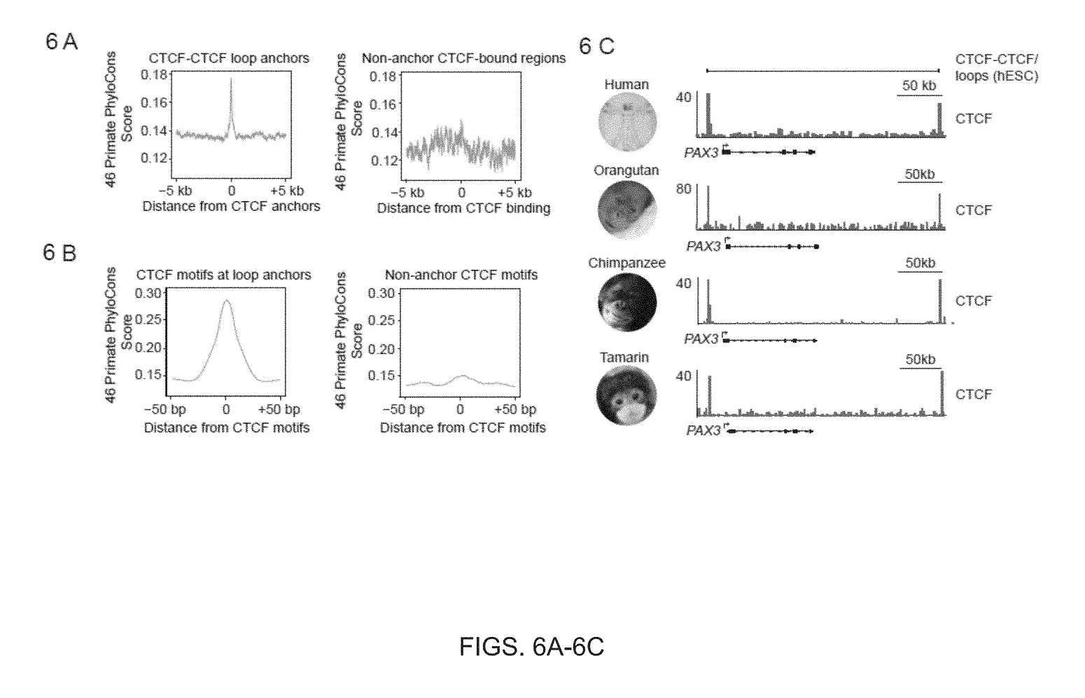

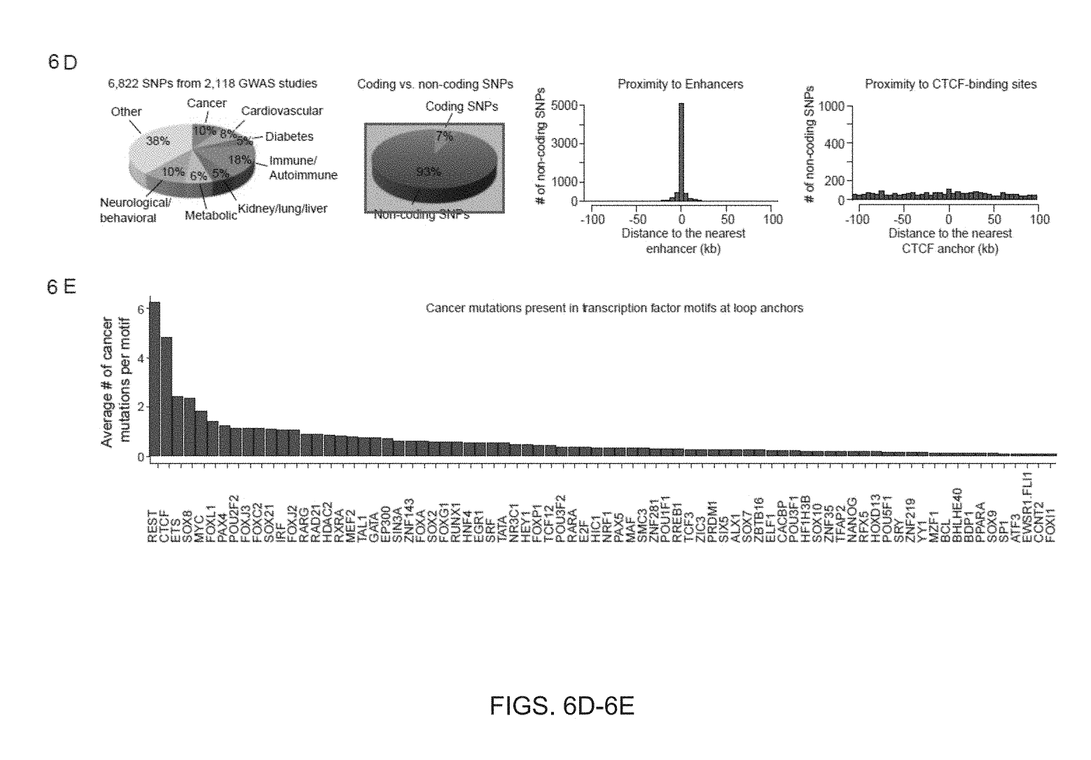

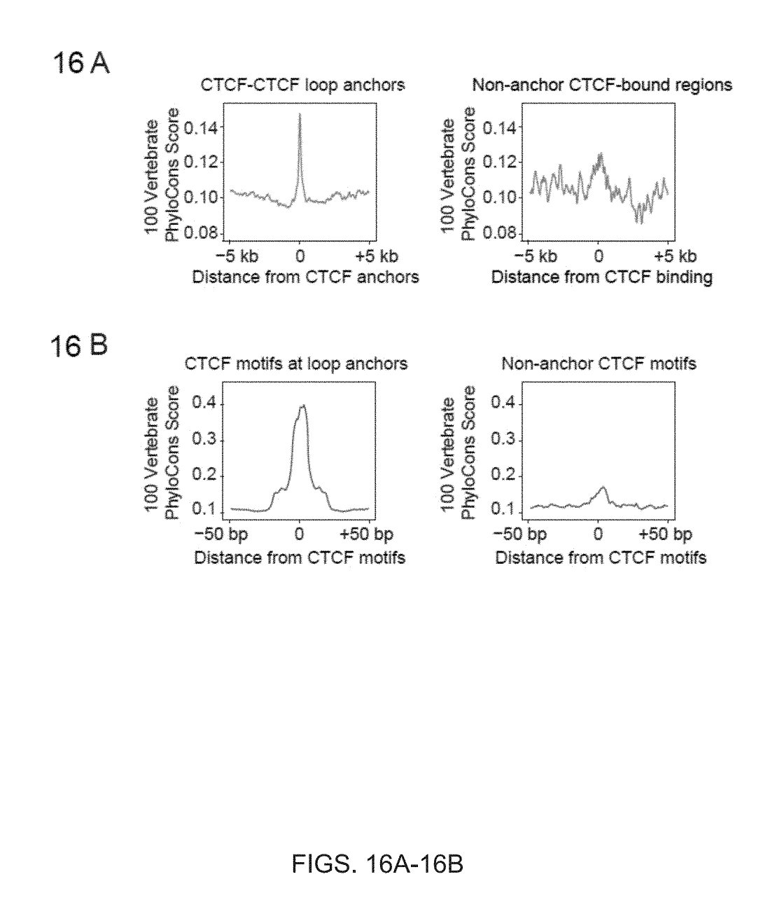

It has been estimated that approximately 65% of Hi-C derived chromosome structures are static among different cell types and different species (Dixon et al., 2015). The observation that chromosome structures are largely conserved in primates (Dixon et al., 2015; Dixon et al., 2012; Pope et al., 2014; Rao et al., 2014; Schmidt et al., 2012; Vietri Rudan et al., 2015) led us to investigate the extent to which CTCF binding is similarly conserved. Analysis of CTCF binding sites across 46 primates indicates that the DNA sequence in anchor regions of CTCF-CTCF loops in hESCs is far more conserved in primates than in regions bound by CTCF that do not participate in loops (FIG. 6A). A similar analysis showed that the CTCF DNA sequence motif in hESC loop anchor regions is highly conserved in primates (FIGS. 6B-6C). Further analysis revealed that the CTCF binding sequences in hESC loop anchor regions are conserved among vertebrates (FIG. 16).

The conservation of CTCF-CTCF loop anchor sequences led us to consider whether their variation contributes to various human diseases and syndromes. Analysis of disease-associated single nucleotide polymorphisms (SNPs) showed that they tend to occur in proximity to enhancers, as observed previously (Hnisz et al., 2013; Maurano et al. 2012), but were not enriched in CTCF-CTCF loop anchor regions that lack evidence of local enhancer activity (FIG. 6D). Deletions, duplications and inversions that affect TAD structure and contribute to congenital diseases have been reported (Giorgio et al., 2015; Ibn-Salem et al., 2014; Lupianez et al., 2015), but these results suggest that disease-associated SNPs generally occur more frequently in enhancers than in CTCF loop anchor regions.

Misregulation of gene expression is a common feature in cancer (Hanahan and Weinberg, 2011; Lee and Young, 2013) and with evidence that proper regulation of gene expression depends on CTCF-CTCF insulated neighborhoods, it is possible that this scaffold is altered in cancer cells. Indeed, a recent report indicates that CTCF/cohesin-binding sites are frequently mutated in colorectal cancer (Katainen et al., 2015). Analysis of somatic mutations present in the International Cancer Genome Consortium database (Zhang et al., 2011) revealed that 8578 mutations occur in hESC CTCF loop anchors (Table S19), and that the CTCF DNA binding motif present in hESC loop anchor regions is among the most altered human factor binding sequence in cancer cells (FIG. 6E). This result suggests that mutations that alter the cohesin-associated CTCF-CTCF loops identified here in hESCs play an important role in the misregulation of gene expression that is inherent to the cancer state.

### DISCUSSION

Described herein is a 3D regulatory landscape of human embryonic stem cells (ESCs) and new insights into the relationship between chromosome structure and gene regulation. Enhancers and genes interact within the context of CTCF-CTCF loops, which thus form insulated neighborhoods that constrain interactions between regulatory elements and genes. TADs appear to be formed by clusters of CTCF-CTCF loops and the gene regulatory interactions that occur within them. Comparison of genetically identical naive and primed ESCs revealed that key differences in gene control occur in the context of similar insulated neighborhoods in the two pluripotent cell states. The CTCF sites that contribute to insulated neighborhoods in hESCs are highly conserved in primates, are rarely affected by sequence variation in humans, but are frequently lost in cancer. These initial 3D regulatory maps of human pluripotent cells thus reveal how cohesin-associated CTCF-CTCF and enhancer-promoter loops contribute to TAD structure and the control of key genes, and provide a foundation for further studies of development and disease.

Several lines of evidence suggest that TADs are dominated by cohesin-associated CTCF-CTCF and enhancer-promoter structures. A large portion of TADs consist of TAD-spanning CTCF-CTCF loops, and much of TAD substructure consists of the CTCF-CTCF loops that are nested within the TAD-spanning loops. The TAD structures that are identified with Hi-C data can also be identified with cohesin-associated loop data by using the same algorithm. All the conserved features that have been described for TADs are also conserved features of cohesin-associated CTCF-CTCF loops.

In their simplest and most conserved form, TADs can be considered as nested sets of cohesin-associated CTCF-CTCF loops, as illustrated by the models shown in FIG. 4. In many cases, the largest CTCF-CTCF loop spans the TAD and additional CTCF-CTCF loops often occur within the TAD. This structure helps explain why enhancers generally control only a limited number of genes despite having an ability to function in either orientation and at long distances, and why only a subset of CTCF-bound sites function as insulators. The pairs of CTCF-bound sites that interact to form a loop can function to produce an insulated neighborhood within which regulatory interactions occur. These results confirm and provide a mechanistic explanation for the hypothesis that TADs provide physical and functional constraints on interactions between regulatory elements and genes (Dekker, 2014; Gorkin et al., 2014).

Most insulated neighborhoods are shared by naive and primed hESCs and likely to be preserved in more differentiated cell types. Although there are substantial differences in regulation of specific genes between naive and primed cells, this differential regulation often occurs within very similar insulated neighborhood structures. Most of these hESC chromosome structures are probably retained during differentiation and thus provide a foundation for further understanding transcriptional control of cell identity in a broad spectrum of human cells, where approximately a million regulatory elements have been mapped but have yet to be physically and functionally linked to genes (Cheng et al., 2014; Kellis et al., 2014; Kundaje et al., 2015; Leung et al., 2015; Thurman et al., 2012). These maps of hESC genome structure should also prove valuable for identifying and further understanding genetic alterations that disrupt 3D structures and cause disease.

### Materials and Methods

### Cell Culture

Primed and naive hESCs were cultured as previously described (Theunissen et al., 2014). Primed hESCs were maintained on mitomycin C-inactivated MEF feeder layers and passaged every 7-10 days. When passaging primed hESCs, clumps of cells were partially dissociated with collagenase type IV (GIBCO, 17104-019), and then subjected to two sedimentation steps in stationary 50 cm tubes for 10 minutes at room temperature in primed hESC medium to remove single cells. Primed hESC medium (500 ml) consisted of 400 ml of Dulbecco's Modified Eagle Medium: Nutrient Mixture F-12 (DMEM/F12, Invitrogen, 11320), 75 ml Fetal Bovine Serum (FBS, Hyclone, SH30071.03HI), 25 ml KnockOut™ Serum Replacement (KSR, Invitrogen, 10828-028), supplemented with 1 mM glutamine (Invitrogen, 25030-024), 1% nonessential amino acids (Invitrogen, 11140-050), penicillin-streptomycin (Invitrogen, 15140-122), 0.1 mM β-mercaptoethanol (Sigma, M6250-100ML), and 4 ng/ml FGF2 (R&D systems, 233-FB-025).

For the induction of naive hESCs, primed hESCs were cultured for 24 hr in the primed hESC medium described above, further supplemented with 10 μM ROCK inhibitor Y-27632 (Stemgent, 04-0012). Colonies were then trypsinized to form a single cell suspension and cells were plated onto a MEF feeder layer in the primed hESC medium+ROCK inhibitor described above. 24 hr later, the medium was switched to 5i/L/A naive hESC medium. The 5i/L/A naive hESC medium (500 ml) used for induction and maintenance of naive hESCs was made up of 240 ml DMEM/F12, 240 ml Neurobasal (Invitrogen, 21103), 5 ml N2 supplement (Invitrogen, 17502048) and 10 ml B27 supplement (Invitrogen, 17504044), supplemented with 10 μg recombinant human LIF (purified in-lab from E. coli), 1 mM glutamine, 1% nonessential amino acids, 0.1 mM β-mercaptoethanol, penicillin-streptomycin, 50 μg/ml BSA (Sigma, A4737-25G), and the following small molecules and cytokines: 1 μM PD0325901 (Stemgent, 04-0006), 1 μM IM-12 (Enzo, BML-WN102-0005), 0.5 μM SB590885 (R&D systems, 2650/10), 1 μM WH-4-023 (A Chemtek) 10 μM Y-27632 (Stemgent, 04-0012), and 10 ng/ml Activin A (Peprotech, 120-14). Following an initial wave of widespread cell death, dome-shaped naive hESC colonies appeared within 10 days and could be expanded and maintained in 5i/L/A naive hESC medium.

Naive hESCs were maintained on mitomycin C-inactivated MEF feeder cells and passaged every 5-7 days. The naive hESCs were passaged by dissociating cells with accutase (GIBCO, A1110501), and then centrifuging cells at 1000 rpm for 5 minutes at room temperature in neutralization medium (DMEM supplemented with 10% FBS, 1 mM glutamine, 1% nonessential amino acids, penicillin-streptomycin, and 0.1 mM (3-mercaptoethanol). To harvest cells for downstream experiments, primed and naive hESCs were trypsinized and subsequently pre-plated on gelatin-coated dishes to deplete MEF feeder cells. All cell culture experiments were performed under physiological oxygen conditions (5% 02, 3% CO2).

### RNA-Seq

RNA-seq was performed for naive and primed hESCs. 6 million cells were used for each RNA extraction. Total RNA was purified using the mirVana™ miRNA Isolation Kit (Life Technologies, AM1560) following the manufacturer's instructions. 1 μg of total RNA was used for the RNA-seq library construction. A technical replicate was performed for both naive and primed hESCs. Polyadenylated RNA-seq libraries were prepared using the TruSeq Stranded mRNA Library Prep Kit (Illumina, RS-122-2101). The RNA-seq libraries were sequenced on the Illumina HiSeq 2000.

### RNA-Seq Expression Analysis

RNA-seq alignment and quantification were performed using the TopHat and Cufflinks software tools. RNA-seq reads were first aligned to the human genome (build hg19, GRCh37) using Tophat v2.0.13 (Trapnell et al., 2009) with the parameters: --solexa-quals--no-novel-juncs and using RefSeq gene annotations. The expression levels of RefSeq transcripts were calculated using Cufflinks v2.2.1 (Trapnell et al., 2010). Differentially expressed transcripts were then identified, again using Cufflinks v2.2.1. When multiple transcripts had the same gene name, only the transcript with the highest expression level was kept for further consideration. A gene was considered differentially expressed if it met the following criteria: 1) absolute log 2 fold-change ≥1 between the mean expression in the two conditions; 2) false discovery rate q-value ≤0.05.

Three lines of evidence suggested that the RNA-seq datasets were high-quality: 1) ˜80% of all reads in all libraries mapped to RefSeq transcript models (hg19), as expected for sequencing of RNA; 2) ˜90% of all reads in all libraries mapped to known RefSeq genes (˜83% mapped to the exons and ˜7% mapped to the introns), as expected for sequencing of poly-A RNA-enriched samples; 3) the replicates of either naive or primed RNA-seq datasets had a Pearson correlation coefficient of expression levels of 0.98 or greater across all RefSeq transcripts.

### Cross-Species Gene Expression Analysis

Cross-species gene expression analysis was performed as previously described (Theunissen et al., 2014). For a given gene, the mean expression value for that gene across all human samples was first calculated. Then for each human sample, the expression of that gene in that sample was divided by the mean expression value. The normalization was repeated for all mouse samples. After normalization, all pairwise comparisons of datasets, both intra- and inter-species, were performed using Pearson correlation coefficients (PCCs). The average linkage hierarchical clustering of the Pearson correlation was shown in the heatmap.

### ChIP-Seq Library Generation and Sequencing

Chromatin immunoprecipitation (ChIP) was performed as previously described (Ji et al., 2015). 50 million naive or primed hESCs were used for each ChIP experiment. The following antibodies were used for ChIP: anti-H3K27ac (Abeam, ab4729), anti-CTCF (Millipore, 07-729), anti-MED1 (Bethyl Labs, A300-793A), anti-OCT4 (Santa Cruz, sc-8628). For each ChIP, 5 μg of antibody and 50 μl protein G Dynabeads (Life technology, 10004D) were used. The ChIP-seq libraries were prepared using the TruSeq CUP Sample Prep Kit (Illumina, IP-202-1012), and sequenced on the Illumina HiSeq 2000.

### ChIA-PET Library Generation and Sequencing

ChIA-PET was performed using a modified version of a previously described protocol (Dowen et al., 2014). 400 million naive or primed hESCs were used for each ChIA-PET library construction. The ChIA-PET libraries were generated in three stages. In the first stage, ChIP was performed using 25 μg anti-SMC1 antibody (Bethyl Labs, A300-055A) and 250 μl protein G Dynabeads (Life technology. 10004D). This stage was the same as the experimental procedure described in the ChIP-seq library generation.

The second stage was proximity ligation of ChIP-DNA fragments, which consists of end blunting and A-tailing to create easily ligated ends, followed by ligation to simultaneously add linker sequences required for later steps and ligate ends of fragments together. The ligation was performed in a large volume to encourage ligation of ends that are in close spatial proximity to each other, ideally from fragments that are co-localized via their interaction with cohesin-bound regions and immunoprecipitation of cohesin. The ChIP-DNA with beads were washed once with TE buffer, then incubated in 1×T4 DNA polymerase buffer (NEBuffer 2.1, New England Biolabs, B7202S), with 7.2 μl T4 DNA polymerase (New England Biolabs, M0203S) and 7 μl of 10 mM dNTPs (Life Technologies, 18427013) in 700 μl total volume at 37° C. for 40 min. The beads were then washed three times with ChIA-PET wash buffer (10 mM Tris-HCl pH7.5, 1 mM EDTA, 500 mM NaCl). The beads were incubated with 1×NEB buffer 2 (New England Biolabs, B7002S) containing 7 μl Klenow fragment (3′-5′ exo⁻) (New England Biolabs, M0212S) and 7 μl 10 mM dATP (New England Biolabs, N0440S) in 700 μl total volume at 37° C. for 50 min. The beads were then washed three times with ChIA-PET wash buffer. The beads were then incubated with 1×T4 DNA ligase buffer with 1 mM ATP (New England Biolabs, B0202S) containing 42 μl T4 DNA ligase (Life Technologies, 46300018) and 4 μl bridge linker (200 ng/μl including Forward: /5Phos/CGCGATATC/iBiodT/TATCTGACT; Reverse: /5Phos/GTCAGATAAGATATCGCGT) in 14 ml total volume at 16° C. for 22 hr. The beads were then washed three times with ChIA-PET wash buffer. The beads were then incubated with 1× lambda exonuclease buffer (New England Biolabs, M0262S) containing 6 μl lambda exonuclease (New England Biolabs, M0262S), and 6 μl exonuclease T (New England Biolabs, M0293S) in 700 μl total volume at 37° C. for 1 hr. DNA elution and crosslink reversal were simultaneously performed by incubating the beads at 55° C. overnight. 10 μl of proteinase K (Life Technologies, AM2546) was included during the overnight incubation. The DNA was then purified by phenol-chloroform extraction and ethanol precipitation.

The third stage was the tagmentation of ligated products, purification of the tagmented. DNA fragments, amplification of the DNA by PCR, size selection and paired-end sequencing. The ChIA-PET proximity ligation products were tagmented with Tn5 Transposase (5 μl Tn5 transposase (Illumina, FC-121-1030) for 50 ng DNA) at 55° C. for 5 min, then at 10° C. for 10 min. DNA was purified using a Zymo column (VWR, 100554-654) following the manufacturer's instructions. Biotin-labeled DNA was then further affinity purified with M280 streptavidin beads (50 μl for each library, Life Technologies, 11205D), followed by washing five times with 2×SSC/0.5% SDS and then two times with 1×B&W buffer (5 mM Tris-HCl pH7.5, 0.5 mM EDTA, 1 M NaCl). The buffer was discarded and the beads were gently resuspended in 30 EB buffer (QIAGEN). 10 μl of the bead slurry was used for PCR amplification. PCR amplification was performed using the Nextera DNA Sample Preparation Kit (Illumina, FC-121-1031) for 10-12 cycles. The DNA was selected for the size range of 300-500 bp and was purified by gel extraction. The ChIA-PET library was subjected to 100×100 paired-end sequencing using Illumina HiSeq 2000.

### SMC1 ChIA-PET Processing

All ChIA-PET datasets were processed with a method adapted from a previously published computational pipeline (Dowen et al., 2014; Li et al., 2010). The output of paired-end sequencing is a set of reads, where each read is identified by a read id and consists of two mates that represent sequence from the ends of a DNA fragment. The raw sequences of each mate of each read were analyzed for the presence of the PET linker barcodes and trimmed using Cutadapt with the parameters “-m 17-a forward=ACGCGATATCTTATCTGACT-a reverse=AGTCAGATAAGATATCGCGT--overlap 10” (Martin, 2011) Specifically, we searched for a stretch of at least 10 bp that matched the linker sequence. Once this sequence was identified, the linker sequence and all sequence immediately 3′ to this sequence was removed. After removal of linker and 3′ sequence, only sequences of at least 17 bp in length were retained. For downstream analysis, all mates from all reads where at least one mate contained the linker sequence, were used. Sequences of mates were separately mapped to the hg19 human genome using Bowtie with the parameters “-k 1-m 1-v 2-p 4--best--strata” (Langmead et al., 2009). These criteria retained only the uniquely mapped mates, with at most two basepair mismatches, for further analysis. Aligned mates were paired using their respective read id's and now considered PETs (paired-end tags). PETs were filtered for redundancy: PETs with identical genomic coordinates and strand information at both ends were collapsed into a single PET. The PETs were further categorized into intrachromosomal PETs, where the two ends of a PET were on the same chromosome, and interchromosomal PETs, where the two ends were on different chromosomes. The sequences from the ends of all PETs were then analyzed for localized enrichment across the genome using MACS 1.4.2 (Zhang et al., 2008) with the parameters 1e-09-no-lambda-no-model--keep-dup=2″. Regions identified with MACS were considered PET peaks.

To identify long-range chromatin interactions, we first removed intra-chromosomal PETs of length <4 kb because these PETs are suspected to originate from self-ligation of DNA ends from a single chromatin fragment in the ChIA-PET procedure (Dowen et al., 2014). We next identified PETs that overlapped with PET peaks at both ends by at least 1 bp. Operationally, these PETs were defined as putative interactions. Applying a statistical model based upon the hypergeometric distribution identified high-confidence interactions, representing high-confidence physical linking between the PET peaks. To do this, for each PET peak, we calculated a) the total number of PETs that overlap with the peak and b) the number of PETs that overlap with the peak and also connect to another peak. A hypergeometric distribution was used to determine the probability of seeing at least the observed number of PETs linking the two PET peaks. The correction p-values were calculated using the Benjamini-Hochberg procedure (Benjamini and Hochberg, 1995) to control for multiple hypothesis testing. Operationally, the pairs of interacting sites with three independent PETs and an FDR ≤0.01 were defined as high-confidence interactions in the SMC1 ChIA-PET merged dataset and with two independent PETs in the individual SMC1 ChIA-PET replicates.

### ChIP-Seq Data Analysis

All ChTP-Seq datasets were aligned to the human genome (build hg19, GRCh37) using Bowtie (version 0.12.2) (Langmead et al., 2009) with the parameters-k 1-m 1-n 2. We used the MACS peak finding algorithm, version 1.4.2 (Zhang et al., 2008) to identify regions of ChIP-seq enrichment over input DNA control with the parameters “--no-model--keep-dup=1”. A p-value threshold for enrichment of 1e-09 was used for H3K27ac, H3K4me3, H3K27me3, MED1 and OCT4 datasets, while a p-value of 1e-07 was used for the CTCF dataset. UCSC Genome Browser (Kent et al., 2002) tracks were generated using the MACS wiggle file output option with parameters “-w-S-space=50”. All gene-centric analyses in human ESCs were performed using human (build hg19, GRCh37) RefSeq annotations downloaded from the UCSC genome browser (genome.ucsc.edu).

Identification of CTCF-CTCF/Cohesin Loops that Define Putative Insulated Neighborhood

Briefly, CTCF-CTCF/cohesin loops were evaluated for putative insulating function by examining the directionality of reads proximal to loop boundaries. One expectation for a loop with insulating function is that, at a loop boundary, interactions originating just upstream of the boundary connect to a distal point located further upstream while interactions originating just downstream of the boundary connect to a distal point located further downstream. Boundaries satisfying this criteria thus have implied functionality in terms of constraining interactions. Adjacent pairs of boundaries satisfying this criteria would thus be candidates for demonstrating insulating function. ChIA-PET interaction directionality preferences were calculated using a method adapted from Hi-C computational analysis (Mizuguchi et al., 2014). Briefly, each chromosome (autosomes and X chromosome) was divided into non-overlapping 40 kb bins. Each intra-chromosomal ChIA-PET interaction (either below or above 4 kb) was then mapped to the matrix comprised of all pairwise combination of bins. Each end of a ChIA-PET interaction contributed signal to its respective bin, thus generating a matrix of interaction frequencies between bins. ChIA-PET directional preference scores were next calculated from these interaction frequency matrices as the log 2 ratio of upstream to downstream contact frequencies for each region i at distances below 400 kb:

Di=log₂(Σⱼ₌₋₁₀^{j=0}C_{i,i+j}/Σⱼ₌₁₀^{j=10}C_{i,i+j}),

in which C is the ChIA-PET interaction frequency matrix.

Putative insulated neighborhoods were operationally defined as intra-chromosomal CTCF-CTCF/cohesin interactions where each end of the interaction displayed a change in directional preference. This type of change in interaction preference between upstream and downstream genomic regions was previously used to computationally define topologically associating domains (Dixon et al., 2012; Nora et al., 2012). To improve the robustness of calculating interaction preferences at the CTCF-occupied peaks at CTCF-CTCF/cohesin interactions, we calculated the average interaction preference at two neighboring bins in the proximity of the CTCF-occupied peaks. Specifically, we first identified the genomic bins where the two ends of CTCF-CTCF/cohesin interactions were located. For each of the 5′ CTCF-occupied PET peaks of these CTCF-CTCF/cohesin interactions, we selected two bins: one located where the 5′ PET peak was located and the other in the immediately neighboring bin in the 3′ direction. For each of the 3′ CTCF-occupied PET peaks at CTCF-CTCF/cohesin interactions, we also selected two bins: one located where the 3′ PET peak was located and the other in the immediately neighboring bin in the 5′ direction. We then filtered for CTCF-CTCF/cohesin interactions whose mean of interaction directional preference between the two bins at their 5′ PET peak was positive (indicating downstream preferences) and mean of interaction directional preference between two bins at their 3′ PET peak was negative (indicating upstream preferences). Since the ChIA-PET interaction frequency matrix was calculated using 40 kb bins, this method allowed us to detect putative insulated neighborhoods greater than 80 kb.

### TAD Model Construction

For models of TAD structures, we show TAD-spanning loops with at least one PET read. We show those insulated neighborhoods that pass the directionality index criteria described above. All non-overlapping insulated neighborhoods are shown. When overlapping insulated neighborhoods are possible, the loop with the most PET reads supporting the interaction was selected for display. When comparing structures encompassing genes that are differentially expressed in naive versus primed hESCs, structures were first identified in the cell type where the gene is expressed. The second cell type was then examined for the presence of a corresponding structure. For simplicity, a subset of genes is displayed with their associated enhancers. Enhancers were defined as stitched H3K27ac MACS peaks (using the ROSE algorithm). The loop with the highest PET reads supporting each enhancer-promoter or enhancer-enhancer interaction was shown (using PET 2).

### ChIA-PET Interaction Heatmap at Insulated Neighborhoods

Cohesin ChIA-PET interactions were displayed to examine the similarity of neighborhoods between naive and primed hESCs. Insulated neighborhoods for naive hESCs were centered and size-normalized. ChIA-PET PET signal (number of uniquely mapped PETs per million uniquely mapped PETs) was then displayed. For comparison, the ChIA-PET signal from primed hESCs for the regions with the same coordinates was displayed.

### CTCF Motif Orientation Analysis at CTCF-CTCF/Cohesin Loops

The location and orientation of the CTCF motifs at CTCF ChIP-seq peaks were identified using the FIMO software package with a default p value threshold of 10̂-4 (Grant et al., 2011; Matys et al., 2006). In the analysis, the canonical CTCF motif from the Jaspar motif database (ID. MA0139.1) was used. The orientation of CTCF motifs at pairs of CTCF ChIP-seq peaks was next determined. For simplicity, we focused on those CTCF-CTCF/cohesin loops where CTCF peaks could be unambiguously assigned to a CTCF motif: each end overlapped a single CTCF ChIP-seq peak by at least 1 basepair and only a single CTCF motif was at the peak. All pairs of CTCF motifs at the two ends of CTCF-CTCF/cohesin ChIA-PET interactions were classified into one of the four possible classes of motif orientations: a convergent orientation (forward-reverse), a divergent orientation (reverse-forward), the same direction on the forward strand (forward-forward) or the same direction on the reverse strand (reverse-reverse).

### Hi-C Interaction Heatmap

To generate a matrix of Hi-C interaction frequencies mapped to a more recent build of the human genome, previously published Hi-C datasets in H1 hESCs (Dixon et al., 2015) were first downloaded from GEO (available on the World Wide Web at subdomain ncbi.nlm. nih.gov/geo/; accession GSM1267196). The raw reads from these datasets were mapped to the human genome build hg19 and filtered as previously described (Imakaev et al., 2012). Corrected contact probability matrices at 40 kb resolution were obtained using the python hiclib library (available on the World Wide Web at sumdomain bitbucket.org/mirnylab/hiclib).

Super-Enhancers in hESCs

Super-enhancers were identified in naive or primed hESCs using ROSE (available on the World Wide Web at subdomain bitbucket.org/young_computation/rose). This code is an implementation of the method used in (Hnisz et al., 2013; Loven et al., 2013). Briefly, regions enriched for H3K27ac signal were identified using MACS. These regions were stitched together if they were within 12.5 kb of each other and enriched regions entirely contained within +/−2 kb from a TSS were excluded from stitching. Stitched regions were ranked by H3K27ac signal therein. ROSE identified a point at which the two classes of enhancers were separable. Those stitched enhancers falling above this threshold were considered super-enhancers.

### SMC1 ChIP-seq Enrichment Heatmap

The heatmaps show the average ChIP-seq or ChIA-PET read density (r.p.m./bp) of different factors at SMC1 occupied regions. Individual ChIP datasets were processed separately and peaks of enriched signal were identified as described above. For SMC1, the genome was binned into 50 bp bins and read density of signal is shown for the 10 kb region representing +/−5 kb from the center of each SMC1-enriched region. Similar read density of signal is shown for each other factor at the corresponding regions shown for the SMC1 dataset.

### Heatmap Representation of High-Confidence ChIA-PET Interactions

ChIA-PET interaction signals relative to the boundaries of CTCF-CTCF/cohesin loops were mapped in a distance-normalized fashion. For each CTCF-CTCF/cohesin loop, we demarcated three regions: loop, upstream, and downstream. For the loop region, the region was divided into 50 equally sized bins. For the upstream region, we selected a region extending upstream of the loop itself. The upstream region's length was set at 20% of the length of the corresponding loop. The upstream region was then divided into 10 equally sized bins. Similarly, for the downstream region, we selected a region extending downstream from the loop for a distance corresponding to 20% of the length of the loop itself, and divided the region into 10 equally sized bins.

To see whether interactions originating within the loop were generally confined within the loop, we first filtered high-confidence interactions in two ways. We required high-confidence interactions to have at least one end in the interrogated region. This removed interactions where both endpoints of the interaction were anchored outside of the region of interest. We removed interactions that had one end at a domain border PET peak and the other end outside of the domain. This removed interactions that originated at a border and had no end within the domain as we did not consider them to be originating within the domain.

The density of the genomic space covered by ChIA-PET interactions in each bin was next calculated as the number of interactions per bin. Interactions within CTCF-CTCF/cohesin loops were considered. The density of ChIA-PET interactions was row-normalized to the row maximum for each domain and the normalized frequency was displayed. Interactions connecting enhancers and promoters were considered and displayed. The density of ChIA-PET interactions was row-normalized to the row maximum for each domain and the normalized frequency was displayed.

### Assignment of Interactions to Regulatory Element

We assigned the PET peaks of interactions to different regulatory elements, including active enhancers, promoters (+/−2 kb of the Refseq TSS), and CTCF ChIP-seq binding sites. Operationally, an interaction was defined as associated with the regulatory element if one of the two PET peaks of the interaction overlapped with the regulatory element by at least 1 base-pair.

Differential H3K27Ac Signal at Enhancer Clusters Between Naive and Primed hESCs

Enhancer clusters were generated to compare enhancer regions between naive and primed hESCs. We first identified the sets of enhancer clusters in naive and primed hESCs using ROSE (available on the World Wide Web at subdomain bitbucket.org/young_computation/rose). Briefly, regions enriched for H3K27ac signal were identified using MACS. These regions were stitched together if they were within 12.5 kb of each other and enriched regions entirely contained within +/−2 kb from a TSS were excluded from stitching Enhancer cluster regions from naive and primed hESCs that overlapped by 1 bp were then merged together to form a representative region that spans the combined genomic region. A total of 24,755 enhancer cluster regions were identified. For each region, the read density in reads per million per base pair (r.p.m./bp) from the replicate data (2 replicate H3K27ac ChIP-seq datasets in naive hESCs and 2 replicate H3K27ac ChIP-seq datasets in primed hESCs) was calculated, and from this the relative read count of each region was obtained by multiplying read density by the length of the region. The edgeR package was used to model technical variation due to noise among duplicate data sets and the biological variation due to differences in signal between naive and primed hESCs (Robinson et al., 2010). Sequencing depth and upper-quartile techniques were used to normalize all 4 datasets together before common and tagwise dispersions were estimated. The statistical significance of differences between naive and primed hESCs was next calculated using an exact test and resulting p values were subjected to Benjamini-Hochberg multiple testing correction (FDR). The final regions with differential H3K27ac signal were required to have the absolute log 2 fold change of normalized H3K27ac signal greater or equal to 2 and FDR less or equal to 0.05.

### Fold Change of H3K27Ac Signal at Super-Enhancer Clusters

In order to quantify the signal changes of super-enhancers between naive and primed hESCs, H3K27ac ChIP-Seq signal was calculated at the set of all enhancer cluster regions considered as super enhancers in at least one condition. Sequencing depth and upper-quartile techniques were used to normalize the H3K27ac ChIP-Seq signal at these super-enhancer clusters using normalization factors derived from the total 24,755 enhancer cluster regions described above. The log 2 fold change of normalized H3K27ac signal was displayed.

### Saturation Analysis of ChIA-PET Library

To determine the degree of saturation within our ChIA-PET library, we modeled the number of sampled putative interactions, which were defined as PETs that overlapped with two PET peaks at both ends by at least 1 bp, as a function of sequencing depth by a two parameter logistic growth model. Intrachromosomal PETs were subsampled at varying depths, and the number of unique putative interactions that they occupied were counted. Model fitting using non-linear least-squares regression suggested that we sampled approximately 45˜50% of the available intrachromosomal PET space.

### Topologically Associating Domain (TAD) Calling

TADs were determined from interaction matrices using the method and code previously described in (Dixon et al., 2012). For cohesin ChIA-PET-based TADs, cohesin ChIA-PET interactions were used to generate interaction matrices by binning the genome into 40 kb bins and counting the number of PETs connecting any two bins. For H1 hESC Hi-C based TADs, H1 hESC Hi-C data previously generated in (Dixon et al., 2015), was realigned, binned into 40 kb bins, and normalized to generate a Hi-C interaction matrix. Parameters from Dixon et al. were retained (an interaction window of 2 Mb and 40 kb for binning interactions). For human samples, the human reference genome (build hg19, GRCh37) was used and for mouse samples, the mm9 mouse reference genome was used.

### Hi-C Vs ChIA-PET Interaction Comparison

Hi-C data was examined to see if the Hi-C data supported predicted ChIA-PET interactions. To do this, H1 hESC Hi-C data was first processed to create an interaction matrix as described above. The subset of the Hi-C interaction matrix that could be directly compared to the available ChIA-PET data was then selected. The interaction scores from the Hi-C matrix were then plotted as a box plot. For comparison, a random distribution of Hi-C interactions was generated and also plotted.

### TAD Spanning Loops: Percentage and Visualization

TADs derived from Hi-C data from H1 hESCs were examined for the presence of CTCF-CTCF/cohesin loops that spanned the entire TAD. TADs and Hi-C interactions were derived as described above. For each TAD, we queried if there was at least one CTCF-CTCF/cohesin loop that connected the upstream and downstream boundaries of the TAD. For this analysis, each boundary was extended by 40 kb both upstream and downstream. A loop was considered spanning if one end was found in the upstream boundary and the other end was found in the downstream boundary. We examined TADs for the number of spanning loops that connected the two boundaries; the percentages of TADs with 1, 2 or 3 spanning loops were reported. For comparison, the analysis was repeated using a set of randomized, shuffled TADs. For the shuffled set, we used the set of H1 Hi-C based TADs but shuffled the chromosome and start site coordinates. Visualization of spanning loops was done using the CRAN-Circlize package (available on the World Wide Web at subdomain cran.r-project.org/web/packages/circlize/index.html).

### TAD Boundary Overlap

To compare the consistency of TADs called using either Hi-C or ChIA-PET data, we asked if boundaries of Hi-C based TAD were frequently co-localized with boundaries of ChIA-PET based TAD calls. To do this, we examined the overlap of the boundaries of TADs called using ChIA-PET data and Hi-C data. For each boundary, we measured the distance of each Hi-C called TAD boundary to the nearest ChIA-PET called TAD boundary. The distribution of distances was then plotted in a histogram.

### Conservation and Disease Analysis

We examined whether the ends of CTCF-CTCF/cohesin loops overlapped with genomic regions of high sequence conservation or genomic regions associated with disease-causing mutations. We began by identifying the CTCF motifs (as described above) that were within the anchor sites of high confidence CTCF-CTCF/cohesin ChIA-PET interactions. We considered two sets of regions, the first being CTCF-CTCF/cohesin anchor sites and the second being CTCF motif sites that are bound by CTCF and within loop anchor sites. For conservation analysis, the 10 kb of sequence around the midpoint of each CTCF-CTCF/cohesin anchor site (+/−5 kb) was used. For each region, for each basepair, the PhastCons score was determined using a 46 way primate multiple alignment (Pollard et al., 2010). This created a vector of PhastCons scores for each region. The vectors for all regions were then averaged and plotted. For association with cancer mutations, the regions described above were overlapped with the coordinates of simple somatic mutations present in cancer from the International Cancer Genome Consortium (ICGC) database (Zhang et al., 2011). For each basepair, the basepair was scored for presence of a mutation. This created a vector of mutation occurrences for each region. The vectors for all regions were then summed and plotted. For association with disease mutations, the regions described above were overlapped with the coordinates of GWAS SNPs (Welter et al., 2014). GWAS SNPs that fell within these regions were reported. All of the analyses were repeated for CTCF motifs. Here, the sequences analyzed included the motif itself plus 200 bp of sequence upstream and downstream.

### GWAS Catalog Parsing and Distance Distribution.

The NHGRI Genome-Wide Association Study (GWAS) database containing SNPs significantly associated with human traits was downloaded Jun. 19, 2015 and parsed as described in (Hnisz et al., 2013). Briefly, trait-associated SNPs with dbSNP identifiers were reproducibly associated with a trait in two independent studies. SNPs were assigned a genomic position using dbSNP build 142. SNPs falling inside RefSeq coding exons were discarded. The distance distribution of trait-associated, noncoding SNPs to the nearest border of a region in the union of 86 enhancer sets defined in (Hnisz et al., 2013) were shown. The distance distribution of trait-associated noncoding SNPs to the nearest border of a CTCF anchor in the union of the naive and primed anchor sites were shown. SNPs within these regions were assigned to the 0 bin.

Transcription Factor Motif Analysis within the Loop Anchors

We determined the average number of mutations found in occurrences of transcription factor motifs that occur in anchor regions. We first downloaded a set of motif instances from (Kheradpour et al., 2013) consisting of sequence motifs, their assignment to transcription factors and their chromosomal location. We next filtered for those motif instances within anchor regions. For each member of the resulting set of motif instances, we counted how many cancer mutations overlapped the motif instance. The counts for all instances assigned to a given factor were summed and divided by the number of instances assigned to that factor. The simple somatic mutations present in cancer were described in the International Cancer Genome Consortium database (Zhang et al., 2011).

### Accession Numbers

Raw and processed sequencing data were deposited in GEO under accession number GSE69647 and are incorporated here by reference.

### Example 2: Activation of Proto-Oncogenes by Disruption of Chromosome Neighborhoods

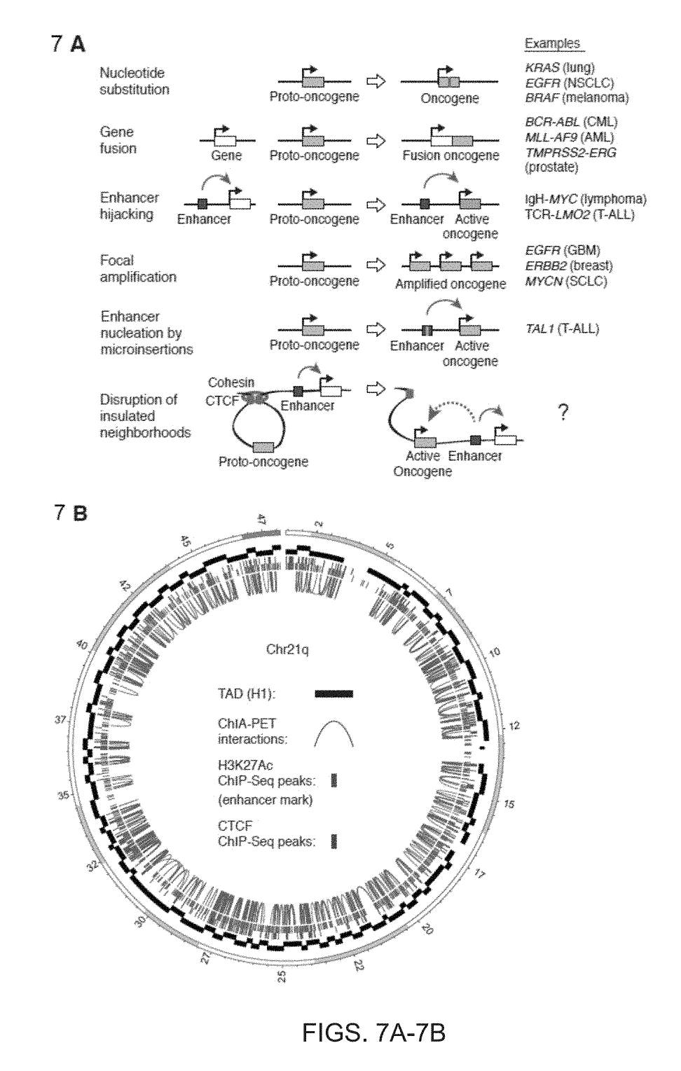

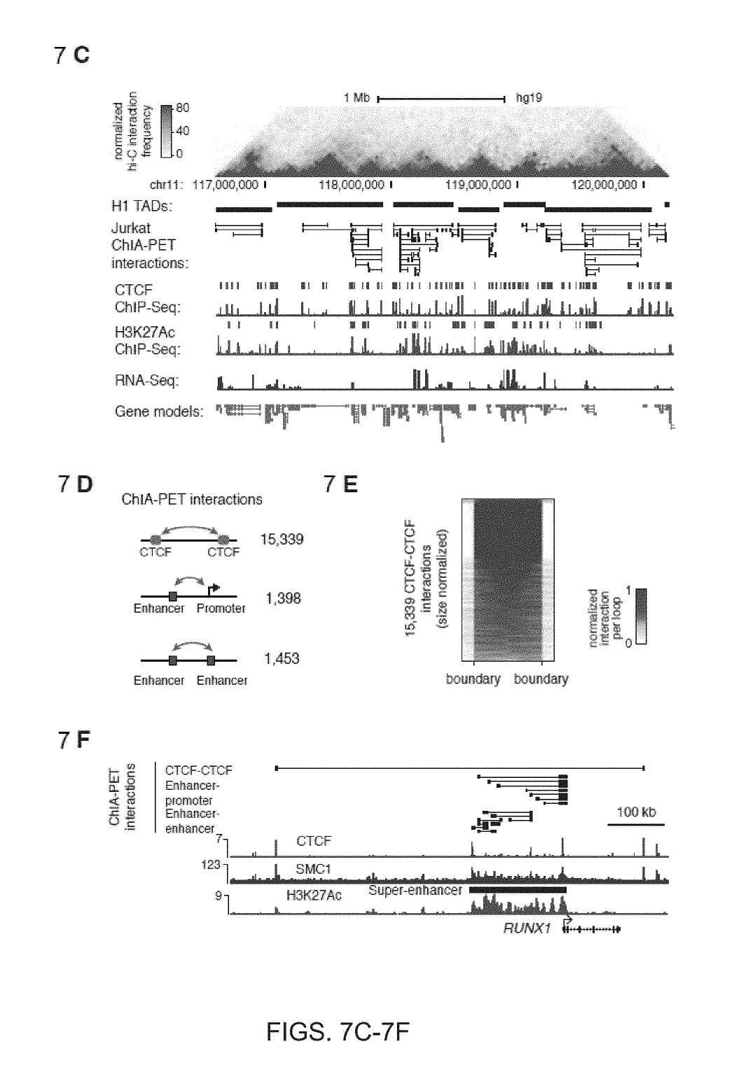

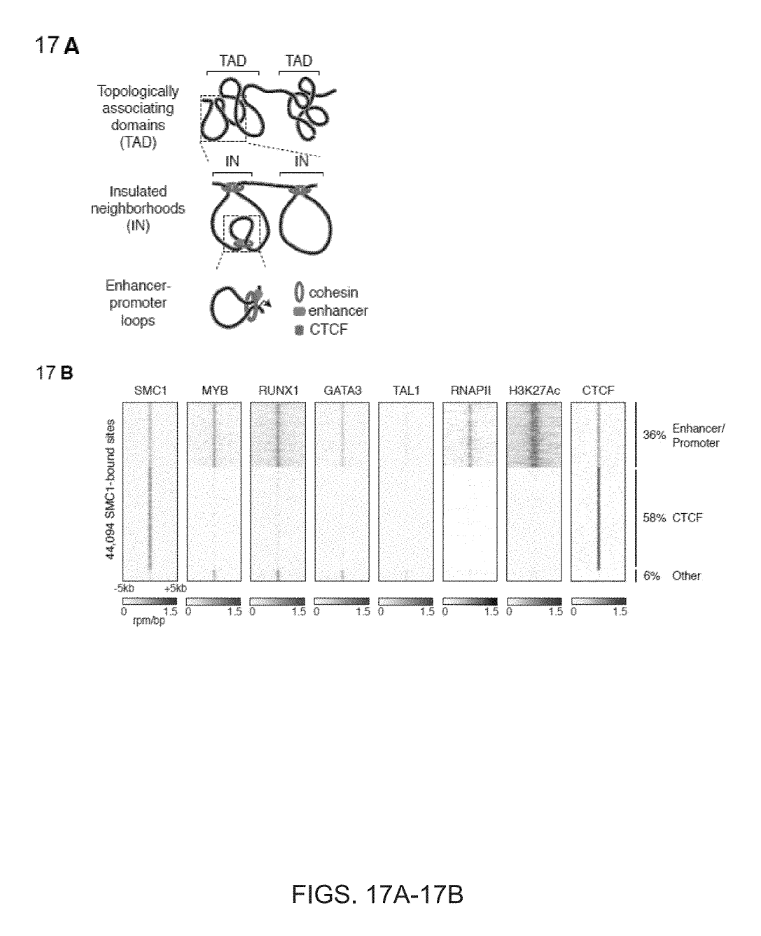

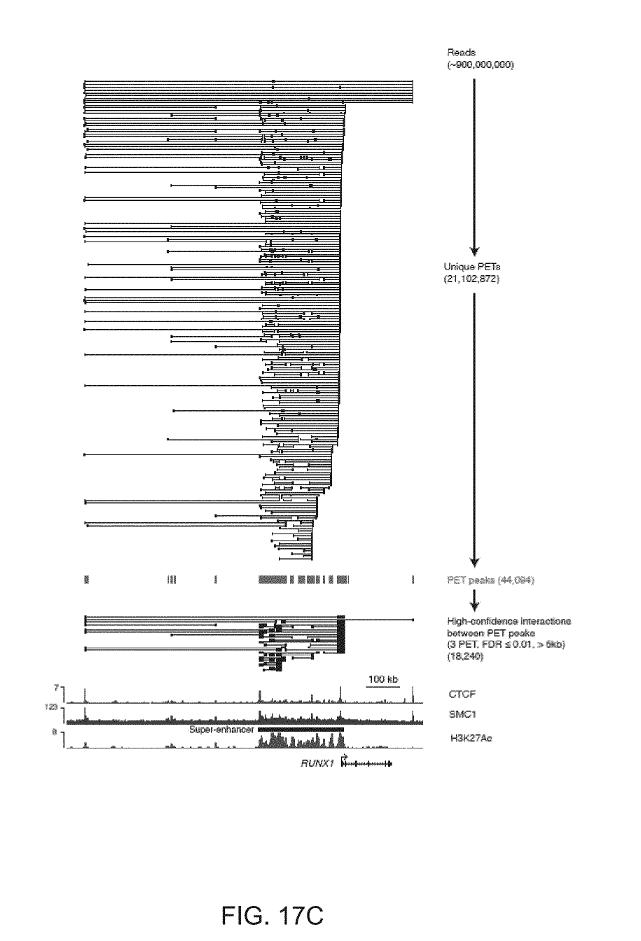

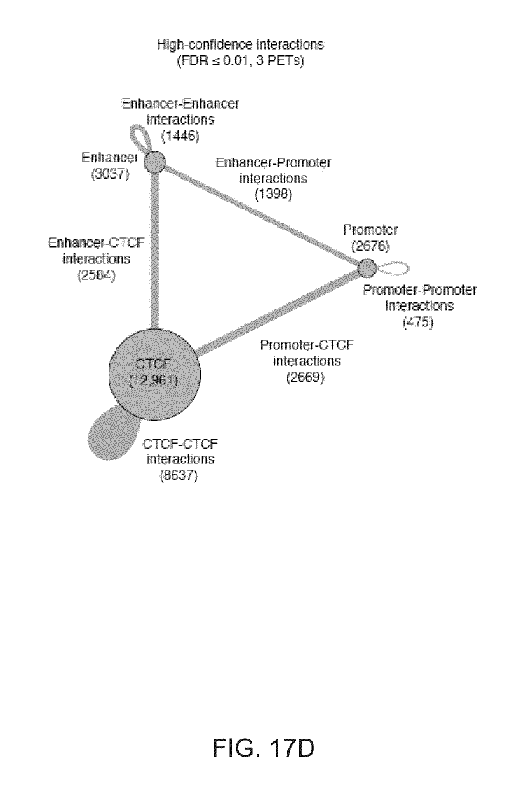

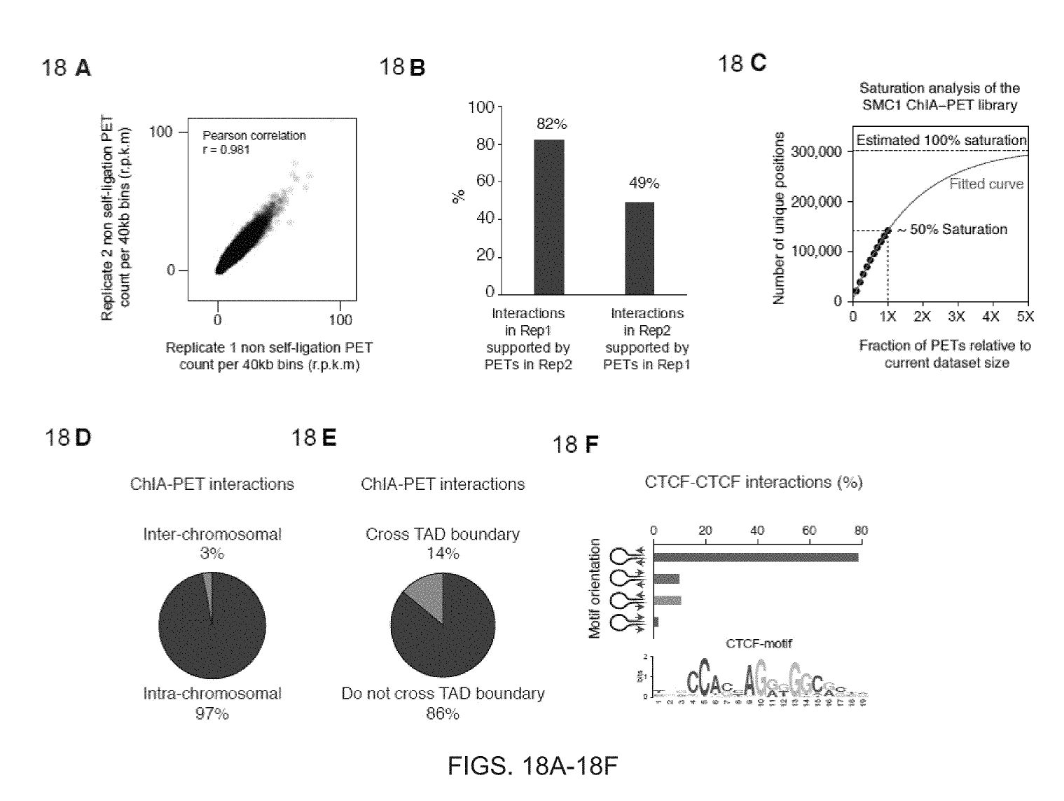

To determine whether proto-oncogenes located within insulated neighborhoods are activated via loss of the neighborhood boundary, insulated neighborhoods and other cis-regulatory interactions in a cancer cell genome were mapped using Chromatin Interaction Analysis by Paired-End Tag Sequencing (ChIA-PET) (FIGS. 7B-7C, Table S1). A T-cell acute lymphoblastic leukemia (T-ALL) tumor cell (Jurkat) was selected for these studies because key T-ALL oncogenes are well-known and genetic alterations that activate T-ALL oncogenes are well-studied (S. A. Armstrong and A. T. Look, J Clin Oncol, 10 Sep. 2005, 23:6306). The ChIA-PET technique was used because it generates a high-resolution (˜4 kb) chromatin interaction map of sites in the genome bound by a specific protein factor (J. M. Dowen et al., Cell, 9 Oct. 2014, 159:374). Cohesin was selected as the target protein because it is involved in both CTCF-CTCF interactions (V. Parelho et al., Cell, 8 Feb. 2008, 32:422) and enhancer-promoter interactions (M. H. Kagey et al., Nature, 23 Sep. 2010, 67:430) is mitotically stable and thus contributes to cellular memory of the gene control program (J. Yan et al., Cell, 15 Aug. 2013, 154, 801), and has proven useful for identifying insulated neighborhoods (J. M. Dowen et al., Cell, 9 Oct. 2014, 159:374) (FIGS. 17A-17B). The replicate cohesin ChIA-PET data, filtered for high-confidence intrachromosomal interactions, revealed 15,339 CTCF-CTCF interactions and 1398 enhancer-promoter interactions (FIG. 7D, FIGS. 17B-17D, FIG. 18A-18F, Table S2). The CTCF-CTCF loops had a median length of ˜240 kb, contained on average 2-3 genes, covered ˜50% of the genome and occurred mostly within the boundaries of previously defined topologically associating domains (TADs) (FIGS. 7B-7C, FIG. 18E, Table S2). The CTCF-CTCF loops are referred to herein as “insulated neighborhoods” because disruption of either CTCF boundary of these CTCF-CTCF loops causes dysregulation of genes located within and/or adjacent to the boundary in every instance tested in embryonic stem cells (J. M. Dowen et al., Cell, 9 Oct. 2014, 159:374). Consistent with the idea that CTCF-CTCF loops also generally function as insulated neighborhoods in Jurkat cells, the majority of other cohesin ChIA-PET interactions (e.g., enhancer-promoter) had endpoints that occurred within these CTCF-CTCF loops (FIG. 7E). Most enhancer-promoter and enhancer-enhancer interactions occurred within these insulated neighborhoods. For example, the RUNX1 oncogene occurs within a 0.5 Mb CTCF-CTCF neighborhood, within which super-enhancer constituents interact with one another and with the RUNX1 promoter (FIG. 7F). These results thus provide an initial map of the 3D regulatory landscape of a tumor cell genome.

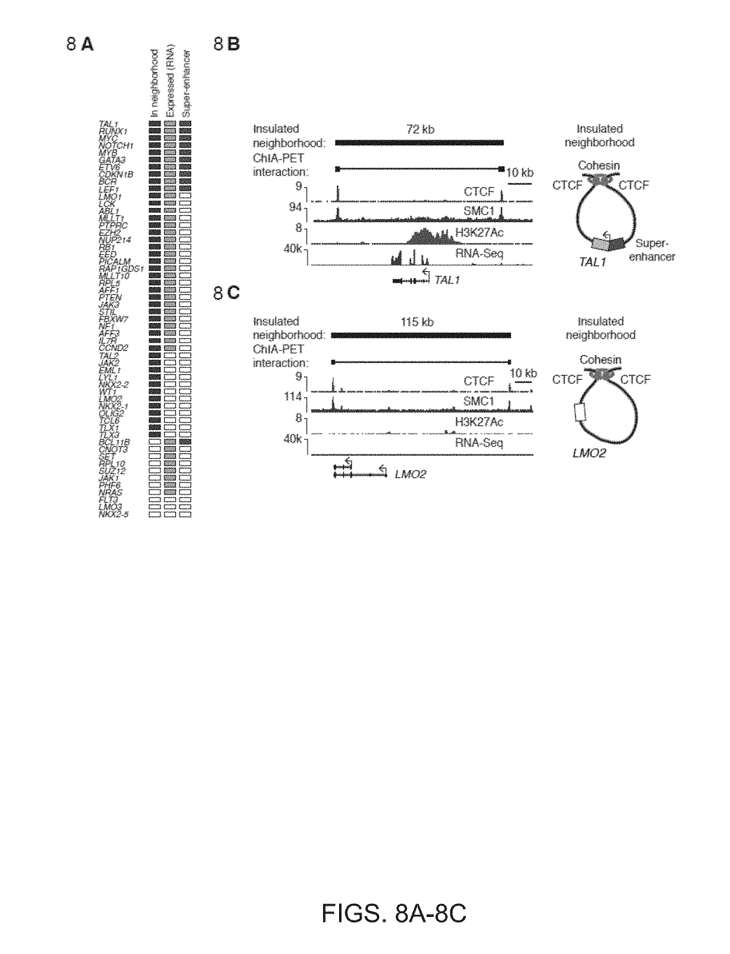

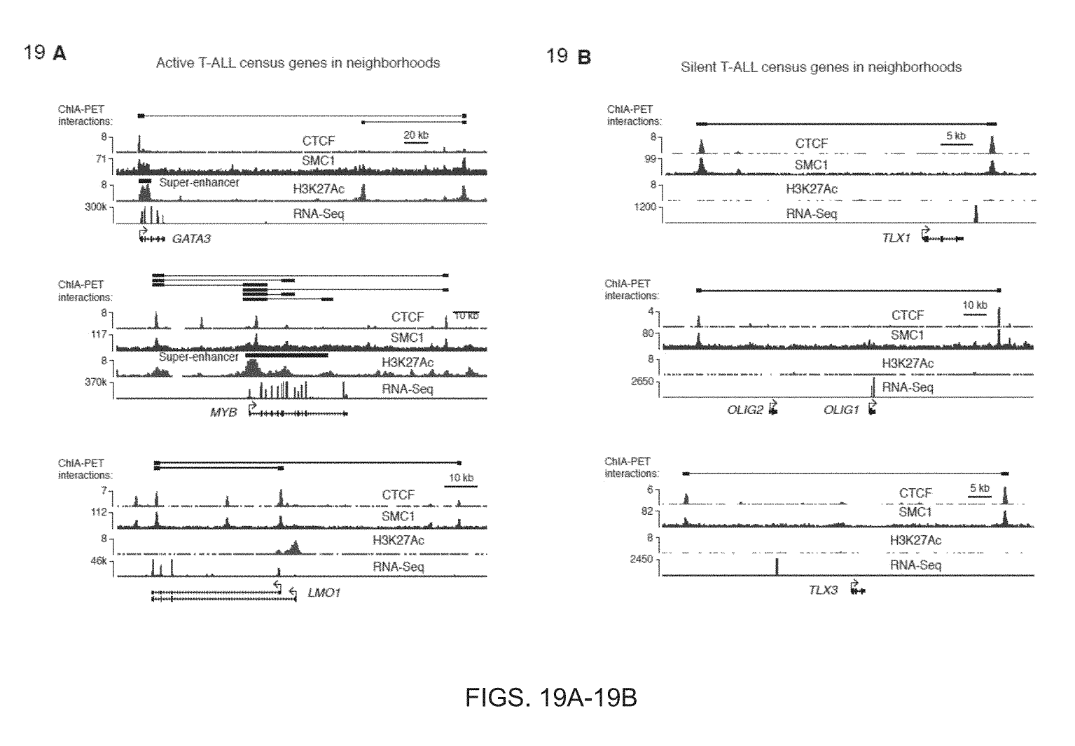

The relationship between genes that have been implicated in T-ALL pathogenesis and the identified insulated neighborhoods was investigated. The majority of genes (44/55) implicated in T-ALL pathogenesis (curated from the Cancer Gene Census and individual studies; referred to as “T-ALL Pathogenesis Genes”) (S. A. Forbes et al., Nucleic Acids Res, January 2015, 43:D805) (Table S3) were located within insulated neighborhoods identified in Jurkat cells (FIG. 8A). Among these 44 genes, 29 were transcriptionally active and 15 were silent based on RNA-Seq data in Jurkat cells (FIG. 8A, Table S4). Active oncogenes can be associated with large clusters of enhancers termed super-enhancers (D. Hnisz et al., Cell, 7 Nov. 2013, 155:934). Ten of the active T-ALL Pathogenesis Genes occurred in an insulated neighborhood together with super-enhancers (FIGS. 8A-8B, FIG. 19A). Silent genes have also been shown to be protected by insulated neighborhoods from active enhancers located outside the neighborhood and multiple instances of silent proto-oncogenes located within CTCF-CTCF loop structures in the Jurkat genome (FIGS. 8A-8C, FIG. 19B) were observed. Both active oncogenes and silent proto-oncogenes are located within CTCF-CTCF loop structures consistent with insulated neighborhoods in these T-ALL cells.

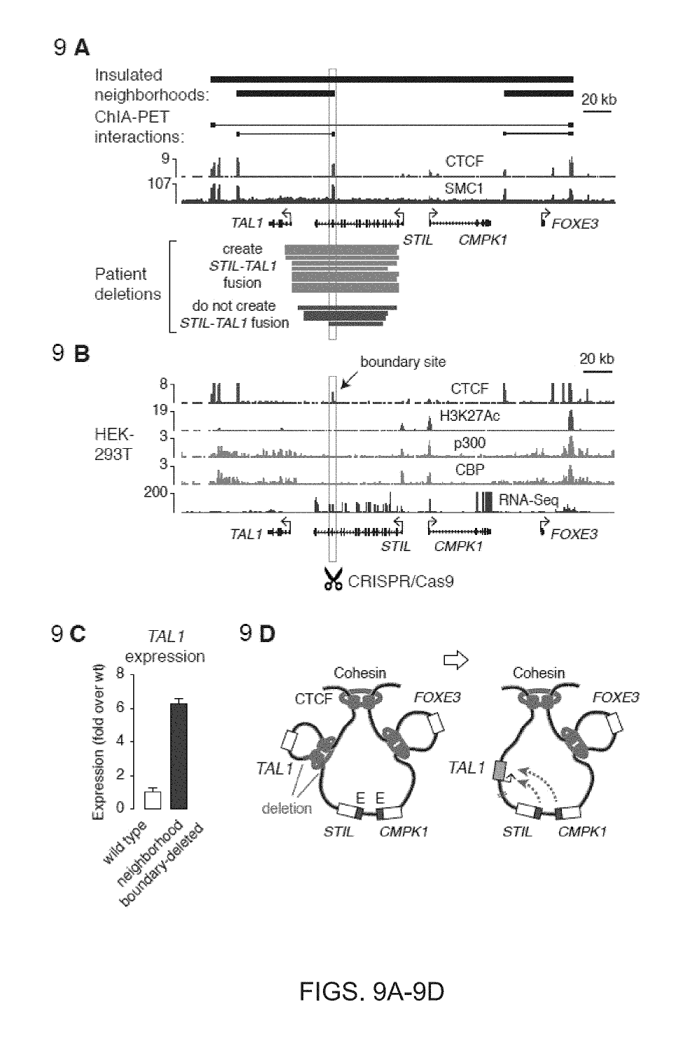

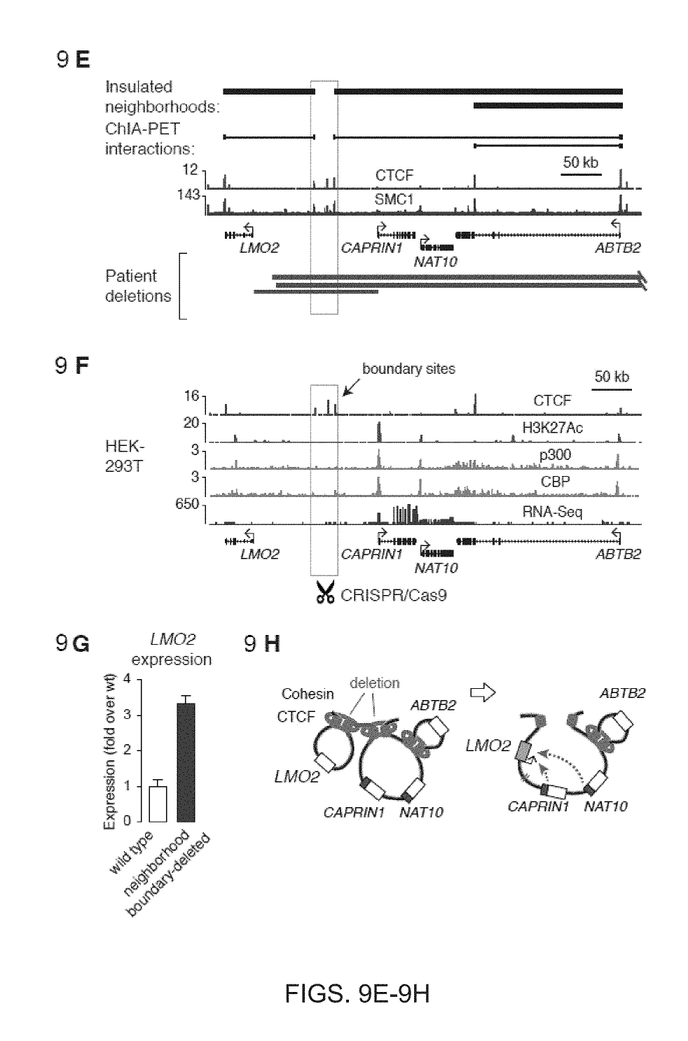

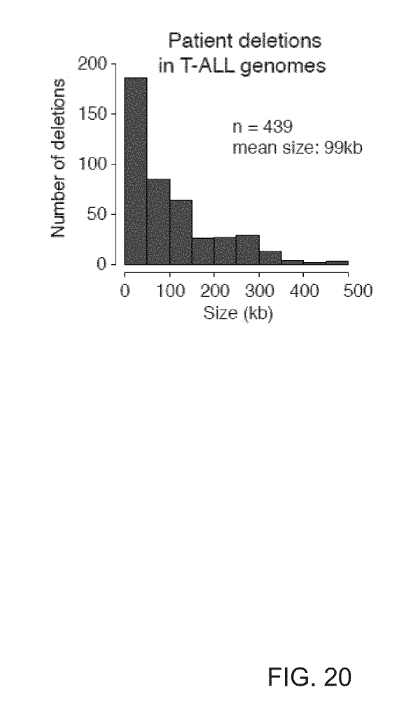

Some insulated neighborhoods function to prevent proto-oncogene activation by enhancers located outside the neighborhood. Some T-ALL tumor cells have genetic alterations that perturb the CTCF boundary elements of neighborhoods containing T-ALL oncogenes. Recurrent deletions in T-ALL genomes that span insulated neighborhood boundaries were identified using data from multiple studies (L. Holmfeldt et al., Nature Genetics, (March 2013), 45:242; C. G. Mullighan et al., Nature, 4 Apr. 2007, 446:758; E. Papaemmanuil et al., Nature Genetics, February 2014, 46:116; and J. Zhang et al., Nature, 12 Jan. 2012, 481:157) and filtered for relatively short deletions (<500 kb) in order to minimize collection of deletions that affect multiple genes (FIG. 20). Among the 439 deletions identified with this approach, 131 overlapped at least one boundary site of the insulated neighborhoods identified in T-ALL and 9 of these affected neighborhoods containing T-ALL Pathogenesis Genes (Table S5). Examples of recurring deletions that overlap neighborhood boundaries of two such genes, TAL1 and LMO2, are shown in FIG. 9A and FIG. 9E.

Deletions that overlap neighborhood boundaries can cause transcriptional activation of proto-oncogenes found within the loops and site-specific deletion of the loop boundary CTCF site activate the respective proto-oncogene in non-malignant cells. TAL1 encodes a transcription factor that is overexpressed in ˜50% of T-ALL cases and is a key oncogenic driver of this cancer (L. Brown et al., EMBO J, October 1990, 9:3343). The TAL1 gene is located in an insulated neighborhood that is nested within a larger neighborhood containing the STIL, CMPK1 and FOXES genes (FIG. 9A). TAL1 is known to be activated in some T-ALL tumor cells by chromosomal deletions that fuse the STIL upstream regulatory region to the first exon of TAL1, thereby creating a STIL-TAL1 fusion (L. Brown et al., EMBO J, October 1990, 9:3343). Analysis of the deletion co-ordinates (J. Zhang et al., Nature Gen., June 2013, 45:602) revealed that several patient deletions did not affect the first exon of TAL1, but did overlap the CTCF boundary site of the TAL1 neighborhood (FIG. 9A). This suggests that such deletions disrupt the insulated neighborhood structure, allowing overexpression of TAL1 by regulatory elements located outside of the loop. It is expected that perturbation of the loop boundary CTCF site in other, non-malignant cells would cause activation of the silent TAL1 proto-oncogene in those cells. In CRISPR/Cas9 mediated deletion of the TAL1 neighborhood boundary in human embryonic kidney cells (HEK-293T), the TAL1 proto-oncogene is silent as evidenced by low H3K27Ac occupancy and RNA-Seq and multiple active regulatory elements found distal to the TAL1 neighborhood boundary as evidenced by high level of occupancy of H3K27Ac and p300/CBP (FIG. 9B). Deletion of a ˜400 bp segment encompassing the boundary CTCF site caused a 6-fold induction of the TAL1 transcript (FIG. 9C). These results indicate that the silent state of the TAL1 proto-oncogene is dependent on the integrity of the insulated neighborhood (FIG. 9D).

A second test of the model indicated that site-specific perturbation of a loop boundary is sufficient to activate a proto-oncogene at the locus containing the LMO2 gene. The LMO2 gene encodes a transcription factor that is overexpressed and oncogenic in some forms of T-ALL (P. Van Vlierberghe, A. Ferrando, J Clin Invest, October 2012, 122:3398) and is located within a CTCF-CTCF insulated neighborhood (FIG. 9E). The LMO2 neighborhood is adjacent to another neighborhood containing the CAPRIN1, NAT10, and ABTB2 genes (FIG. 9E). The region upstream of the LMO2 promoter is recurrently deleted in T-ALL and these deletions are linked to LMO2 activation; a previous study proposed that deletion of cryptic repressors located in the deleted region enable activation of LMO2 (P. Van Vlierberghe et al., Blood, 15 Nov. 2006, 108:3520). This locus is of particular interest, because LMO2 upstream deletions (del(11)(p12p13) and (del(11)(p12p12)) occur in multiple cancers, including T-ALL, AML, Wilms tumor and rhabdoid tumor (Cancer Genome Anatomy Project, NCI). Analysis of the deletion breakpoints from a T-ALL patient cohort (J. Zhang et al., Nat Genet, June, 2013 45:602) revealed that the deletions overlap the CTCF boundary site of the LMO2 neighborhood and the adjacent nested neighborhood (FIG. 9E). This suggests that the deletions activate LMO2 through the disruption of the neighborhood structure, which in turn would allow regulatory elements located outside of the loop to activate LMO2. CRISPR/Cas9 mediated deletion of the neighborhood boundaries in human embryonic kidney cells (HEK-293T) indicated that the LMO2 proto-oncogene is silent, and the CAPRIN1, CMPK1 and ABTB2 genes are active as evidenced by H3K27Ac occupancy and RNA-Seq, and multiple active regulatory elements are found distal to the LMO2 neighborhood boundary as evidenced by H3K27Ac and p300/CBP occupancy (FIG. 9F). CRISPR/Cas9-mediated deletion of the ˜25 kb segment encompassing the boundary CTCF sites caused a 3-fold induction of the LMO2 transcript (FIG. 9G). The deleted CTCF sites help maintain the silent state of the 1,11102 proto-oncogene (FIG. 9H).

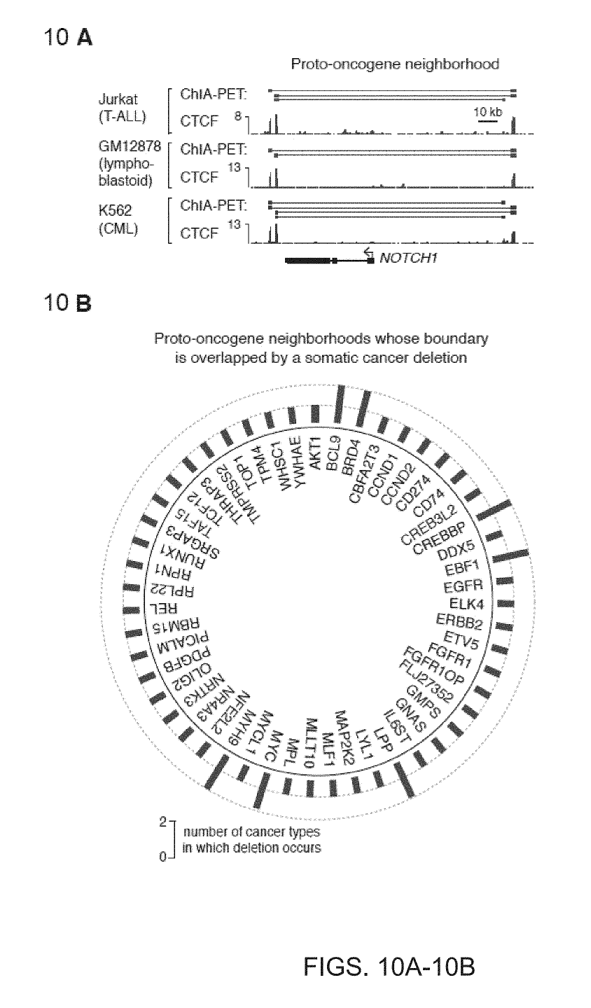

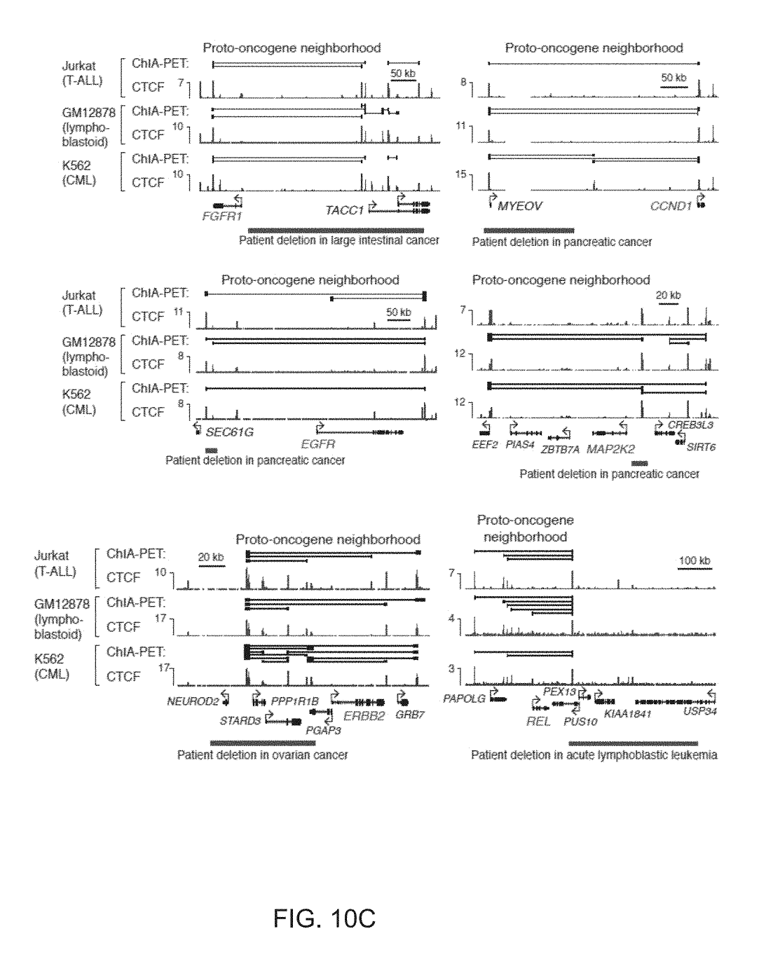

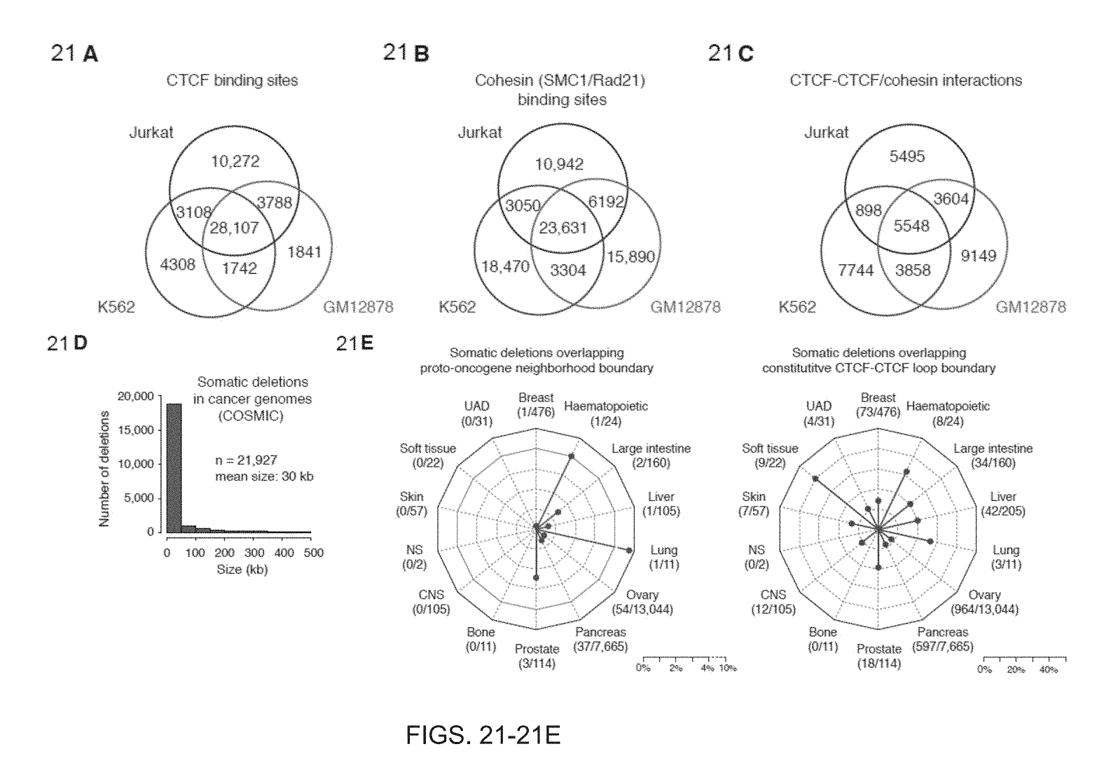

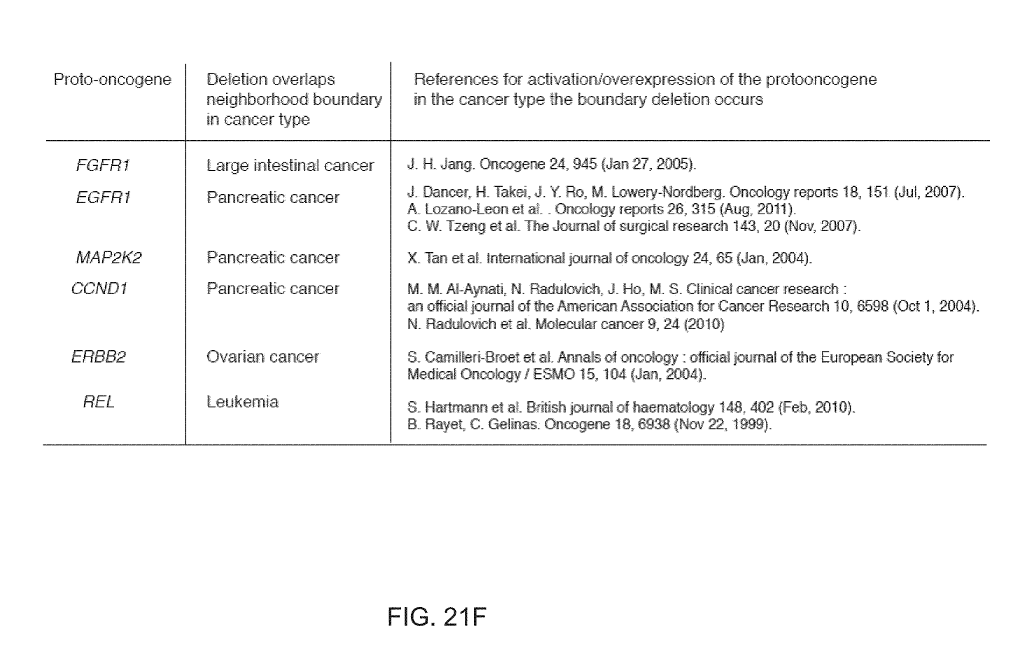

A survey of cancer cell genome sequence data indicates that proto-oncogene neighborhood boundaries are disrupted by small deletions in many types of cancer. To identify proto-oncogene neighborhoods whose boundary is disrupted in cancer genomes, a set of candidate neighborhoods comprised of CTCF-CTCF loops that appeared constitutive across multiple cell types were identified. Previous studies have established that CTCF-cohesin bound sites are largely preserved in multiple cell types (J. M. Dowen et al., Cell, (9 Oct. 2014), 159:374; J. E. Phillips-Cremins et al., Cell, 6 Jun. 2013, 153:1281; and T. H. Kim et al., Cell, (23 Mar. 2007), 128:1231). A similar set of constitutive CTCF-CTCF loops could be detected by comparing CTCF-CTCF interactions detected in Jurkat cells, GM12878 lymphoblastoid and K562 CML cells. (FIGS. 21A-21C, Table S6). After identifying a set of constitutive CTCF-CTCF loops, the genes contained within these loops were compared to a list of proto-oncogenes in the Cancer Gene Census (Table S7) and loops containing proto-oncogenes were considered “proto-oncogene neighborhoods” (FIG. 10A, Table S8). Over two-thirds of proto-oncogenes (224/329) were located in proto-oncogene neighborhoods. The boundaries of the proto-oncogene neighborhoods were then examined for microdeletions found in cancer genomes, using the COSMIC database (available on the World Wide Web at subdomain sanger.ac.uk/cosmic) (FIG. 21D). The boundaries of ˜25% (51/224) of proto-oncogene neighborhoods overlap by a microdeletion in one or more cancers (FIG. 10B) and over half of the cancer types examined (8/14) had at least one proto-oncogene neighborhood boundary overlapped by a deletion (FIG. 21E, Tables S9 and S10). Examples of such proto-oncogene neighborhoods are shown in FIG. 10C, and in all these cases the activation of the gene located in the neighborhood has previously been documented in the respective cancer type (FIG. 21F). These results suggest that somatic deletions that overlap proto-oncogene neighborhood boundaries occur in the genomes of many different cancers.

Disruption of insulated neighborhoods is a genetic mechanism that can cause oncogene activation in malignant cells. An understanding of 3D chromosome structure and its control is rapidly advancing and should be considered for potential diagnostic and therapeutic purposes. With maps of 3D chromosome structure, cancer genome analysis can consider how recurrent deletions at boundary elements may impact oncogene expression. Because control of 3D chromosome structure involves binding of specific sites by CTCF and cohesin, which is affected by protein cofactors, DNA methylation and local RNA synthesis, future advances in our understanding of these regulatory processes may provide new approaches to therapeutics that impact aberrant chromosome structures.

### Materials and Methods

### Cell Culture

Jurkat T-ALL cells were cultured in RPMI GlutaMAX (Invitrogen, 61870-127), supplemented with 10% fetal bovine serum, 100 U/ml penicillin and 100 μg/ml streptomycin (Invitrogen, 15140-122). HEK-293T cells were cultured in DMEM (high glucose, pyruvate; Invitrogen, 11995-073) supplemented with 10% fetal bovine serum, 100 U/ml penicillin and 100 mg/ml streptomycin (Invitrogen, 15140-122).

### ChIP-Seq

ChIP was performed as described in T. 1. Lee, S. E. Johnstone and R. A., Nature Proto., 2006, 1:729 with a few adaptations. Jurkat cells (˜100 million cells, grown to a density of ˜1 million cells/ml) were crosslinked for 10 min. at room temperature by the addition of one-tenth of the volume of 11% formaldehyde solution (11% formaldehyde, 50 mM HEPES pH 7.3, 100 mM NaCl, 1 mM EDTA pH 8.0, 0.5 mM EGTA pH 8.0) to the growth media followed by 5 min. quenching with 125 mM glycine. Cells were washed twice with PBS, then the supernatant was aspirated and the cell pellet was flash frozen in liquid nitrogen. Frozen cross-linked cells were stored at −80° C. 100 ul of Protein G Dynabeads (Life Technologies, Grand Island, N.Y., US) were blocked with 0.5% BSA (w/v) in PBS. Magnetic beads were bound with 10 μg of anti-H3K27Ac antibody (Abcam ab4729), anti-CTCF antibody (Millipore 07-729), anti-RUNX1 antibody (Abeam ab23980) or anti-GATA3 (Santa Cruz sc-22206X) antibody. Nuclei were isolated as previously described in T. I. Lee, S. E. Johnstone and R. A., Nature Proto., 2006, 1:729, and sonicated in lysis buffer (20 mM Tris-HCl pH 8.0, 150 mM NaCl, 2 mM EDTA pH 8.0, 0.1% SDS, and 1% Triton X-100) on a Misonix 3000 sonicator for 10 cycles at 30 sec. each on ice (18-21 W) with 60 sec. on ice between cycles. Sonicated lysates were cleared once by centrifugation and incubated overnight at 4° C. with magnetic beads bound with antibody to enrich for DNA fragments bound by the indicated factor. Beads were washed with wash buffer A (50 mM HEPES-KOH pH7.9, 140 mM NaCl, 1 mM EDTA pH 8.0, 0.1% Na-Deoxycholate, 1% Triton X-100, 0.1% SDS), B (50 mM HEPES-KOH pH7.9, 500 mM NaCl, 1 mM EDTA pH 8.0, 0.1% Na-Deoxycholate, 1% Triton X-100, 0.1% SDS), C (20 mM Tris-HCl pH8.0, 250 mM LiCl, 1 mM EDTA pH 8.0, 0.5% Na-Deoxycholate, 0.5% IGEPAL C-630 0.1% SDS) and D (TE with 50 mM NaCl) sequentially. DNA was eluted in elution buffer (50 mM Tris-HCL pH 8.0, 10 mM EDTA, 1% SDS). Cross-links were reversed overnight. RNA and protein were digested using RNase A and Proteinase K, respectively and DNA was purified with phenol chloroform extraction and ethanol precipitation. Purified ChIP DNA was used to prepare Illumina multiplexed sequencing libraries. Libraries for Illumina sequencing were prepared following the Illumina TruSeq DNA Sample Preparation v2 kit. Amplified libraries were size-selected using a 2% gel cassette in the Pippin Prep system from Sage Science set to capture fragments between 200 and 400 bp. Libraries were quantified by qPCR using the KAPA Biosystems Illumina Library Quantification kit according to kit protocols. Libraries were sequenced on the Illumina Hi Seq 2500 for 40 bases in single read mode.

### ChIP-Seq Data Analysis

ChIP-Seq datasets were aligned using Bowtie (version 0.12.2) (B. Langmead, et al., Genome Rio, 2009, 10:R25) to the human genome (build hg19, GRCh37) with parameter-k 1-m 1-n 2. We used the MACS version 1.4.2 (model-based analysis of ChIP-seq) (Y. Zhang et al., Genome Bio, 2008, 9:R137) peak finding algorithm to identify regions of ChIP-seq enrichment over input DNA control with the parameter “--no-model--keep-dup=1”. A p-value threshold of enrichment of 1e-09 was used for both H3K27Ac and CTCF. UCSC Genome Browser tracks were generated using MACS wiggle outputs with parameters “-w-S-space=50”. The browser snapshots of the ChIP-Seq binding profiles displayed throughout the study use read per kilobase per million mapped reads dimension on the y-axis, except for the SMC1 (cohesin) track which indicates the number of reads in the dataset.

### ChIP-Seq Enrichment Heatmap

ChTP-seq read density (rpm/bp) of SMC1, MYB, RUNX1, GATA3, TALL RNAPII, H3K27Ac and CTCF at the SMC1-bound regions are displayed on FIG. 17B. The input-subtracted average ChIP-seq read density in 50 bp bins was calculated +/−5 kb around the center of the SMC1-enriched regions exactly as previously described in J. M. Dowen, Cell, 9 Oct. 2014, 159:374.

### ChIA-PET

ChIA-PET was performed using a modified version of a previously described protocol (J. M. Dowen, Cell, 9 Oct. 2014, 159:374. Jurkat cells (up to 500-800 million cells, grown to a density of ˜1 million cells/ml) were cross-linked with 1% formaldehyde at room temperature for 10 min. and then neutralized with 125 mM glycine. Cross-linked cells were washed three times with ice-cold PBS, snap-frozen in liquid nitrogen, and stored at −80° C. before further processing. Nuclei were isolated as previously described (T. I. Lee, S. E. Johnstone and R. A., Nature Proto., 2006, 1:729), and chromatin was fragmented using a Misonix 3000 sonicator. The anti-SMC1 antibody (Bethyl, A300-055A) was used to enrich SMC1-bound chromatin fragments exactly as described at the ChIP-Seq section. A portion of ChIP DNA was eluted from antibody-coated beads for concentration quantification and for enrichment analysis using quantitative PCR. For ChIA-PET library construction ChIP DNA fragments were end-repaired using T4 DNA polymerase (NEB) followed by A-tailing with Klenow (NEB). A biotinylated bridge linker (F: /5Phos/CGCGATATC/iBiodT/TATCTGACT; R: /5Phos/GTCAGATAAGATATCGCGT) with T-overhangs was added and the proximity ligation was performed overnight at 16° C. in 1.5 mL volume. Un-ligated DNA was then digested with exonuclease and lambda nuclease treatment (NEB). DNA was eluted off the beads in elution buffer (50 mM Tris-HCL pH 8.0, 10 mM EDTA, 1% SDS) followed by overnight crosslink reversal, RNAse A treatment, and proteinase K treatment. A phenol:chloroform:isoamyl alcohol extraction was performed followed by an ethanol precipitation. Precipitated DNA was re-suspended in Nextera DNA resuspension buffer (Illumina). The DNA was then tagmented with the Nextera Tagmentation kit (Illumina). The tagmented library was purified with a Zymo column and 12 cycles of the polymerase chain reaction were performed to amplify the library. The amplified library was size-selected (350-500 bp) with a Pippen prep machine and sequenced with either 100×100 (Replicate 1) or 125×125 (Replicate 2) paired-end sequencing on an Illumina Hi-Seq 2500.

### ChIA-PET Data Analysis

All ChIA-PET datasets were processed with a method adapted from previous computational pipeline (J. M. Dowen, Cell, 9 Oct. 2014, 159:374). Image analysis and base calling was done using the Solexa pipeline. Reads were examined for the presence of at least 10 base pairs of linker sequence. Reads that did not contain linker were not processed further. Reads containing linker were trimmed using cutadapt (cutadapt-m 17-a forward=ACGCGATATCTTATCTGACT-a reverse=AGTCAGATAAGATATCGCGT-overlap 10) (available on the World Wide Web at subdomain code.google.com/p/cutadapt/). Trimmed mate pairs were mapped independently to hg19 using Bowtie version 1.1.1 (bowtie-e 70-k 1-m 1-v 2-p 4--best--strata-S) (B. Langmead et al., Genome Bio, 2009, 10:R25). Aligned reads were paired with mates with an in-house script using read identifiers. To remove PCR bias artifacts, reads were filtered for redundancy: PETs with identical genomic coordinates and strand information at both ends were collapsed into a single PET. The PETs were further categorized into intrachromosomal PETs or interchromosomal PETs. Regions of local enrichment (PET peaks) were called using MACS 1.4.2 with the parameters “-p 1e-09-no-lambda-no-model”. To identify long-range chromatin interactions, intrachromosomal PETs of length <5 kb were removed because these PETs may originate from self-ligation of DNA ends from a single chromatin fragment in the ChIA-PET procedure (J. M. Dowen, Cell, 9 Oct. 2014, 159:374). PETs that overlapped with PET peaks at both ends by at least 1 bp were identified. These PETs were defined as putative interactions. Applying a statistical model based upon the hypergeometric distribution identified high-confidence interactions, representing high-confidence physical linking between the PET peaks. Specifically, the numbers of PET sequences that overlapped with PET peaks at both ends as well as the number of PETs within PET peaks at each end were counted. The PET count between two PET peaks represented the frequency of the chromatin interaction between the two genomic locations. A hypergeometric distribution was used to determine the probability of seeing at least the observed number of PETs linking the two PET peaks. A background distribution of interaction frequencies was then obtained through the random shuffling of the links between two ends of PETs, and a cutoff threshold for calling significant interactions was set to the corresponding p-value of the most significant proportion of shuffled interactions (at an FDR of 0.01). This method yielded similar number of interactions as the correction of p-values by the Benjamini-Hochberg procedure to control for multiple hypothesis testing. Operationally, the pairs of interacting sites with three independent PETs were defined as high-confidence interactions in the SMC1 ChIA-PET merged dataset and with two independent PETs in the individual SMC1 ChIA-PET replicates.

The RAD21 (cohesin) ChIA-PET datasets in GM12878 and K562 were described in a previous study (N. Heidari et al., Genome Res, December 2014, 24:1905) and were processed exactly as described (J. M. Dowen, Cell, 9 Oct. 2014, 159:374).

### ChIA-PET Replicate Comparison

For the comparison of ChIA-PET replicates displayed on FIG. 18A, all unique intrachromosomal PETs from each dataset were binned into 40 kb bins generating a symmetric m by n matrix for each chromosome. For each dataset a vector was created containing the number of unique intrachromosomal PETs of each genomic position (every bin of the lower triangle of each chromosome matrix). Each vector was then normalized to the total number of unique intrachromosomal PETs for that dataset (the sum of the vector) and multiplied by 1 million to give a normalized interaction frequency. The Pearson correlation was then calculated (r=0.981) and a scatter plot was generated displaying the normalized interaction counts for each replicate.

### ChIA-PET Saturation Analysis

To determine the degree of saturation of the merged ChIA-PET library (FIG. 18C), the number of sampled genomic positions as a function of sequencing depth were modeled (J. M. Dowen, Cell, 9 Oct. 2014, 159:374). Unique intrachromosomal PETs that overlapped with a PET peak on both ends and with a distance span above the self-ligation cutoff of 5 kb were subsampled at varying depths, and the number of unique genomic positions (defined as the start and end coordinates of the paired PETs) that they occupy were counted. Subsampling was performed three times, and the mean values were used to generate the plot on FIG. 18C. A first-order exponential model was fitted and the curve suggests that approximately 50% of the available intrachromosomal PET space was sampled, encompassing 142,312/308,232 positions (FIG. 18C).

### CTCF Motif Orientation Analysis

The DNA binding site of CTCF is asymmetric. Convergent orientation of CTCF motifs is predictive of CTCF-CTCF loop formation (S. S. Rao et al., Cell, 18 Dec. 2014, 159:1665). The motif orientation of the CTCF sites connected by ChIA-PET interactions in the dataset were investigated and a majority (˜80%) of interacting CTCF-CTCF sites demonstrated a convergent motif orientation (FIG. 18F) suggesting high quality of the ChIA-PET data.

FIMO was first used to identify the location and orientation of the CTCF motifs at CTCF ChIP-seq peaks at a default p value threshold of 10̂-4 (C. E. Grant, et al., Bioinformatics, 1 Apr. 2011, 27:1017). The canonical CTCF motif from the Jaspar motif database (ID. MA0139.1) was used. The information of CTCF motif orientation at CTCF ChIP-seq peaks was next overlaid with PET peaks at two ends of CTCF-CTCF/cohesin ChIA-PET interactions. For simplicity, only the CTCF ChIP-seq peaks having a single CTCF motif were used for the analysis and only the CTCF-CTCF/cohesin ChIA-PET interactions were used whose ends overlapped with only a single CTCF ChIP-seq peak by at least 1 base-pair at each end. The pairs of CTCF motifs at the two ends of CTCF-CTCF/cohesin ChIA-PET interactions were then classified into one of the four possible classes of motif orientation: a convergent orientation (forward-reverse), a divergent orientation (reverse-forward), the same direction on the forward strand (forward-forward) or the same direction on the reverse strand (reverse-reverse) (FIG. 18F).

### Hi-C Data Analysis

Previously published Hi-C datasets in H1 human ESCs (J. R. Dixon et al., Nature, 17 May 2012, 485:376) were downloaded from GEO (GSM1267196). The raw reads from these datasets were mapped to the human genome build hg19 and filtered as previously described (M. Imakaev et al., Nature Meth, October 2012, 9:999). Corrected contact probability matrices at 40-kb resolution were obtained using the hiclib library (available on the World Wide Web at subdomain bitbucket.org/mirnylab/hiclib). The corrected contact probability matrices displayed on the heatmap on FIG. 8B were generated by the image function in R.

### Identification of Enhancers and Super-Enhancers

Super-enhancers and typical enhancers in Jurkat cells were identified using H3K27Ac ChIP-Seq data as previously described (D. Hnisz et al., Cell, 7 Nov. 2013, 155:934). Enhancers were defined using H3K27Ac binding sites that were identified using MACS. To identify super-enhancers, enhancers were stitched together if they were within 12.5 kb, and the stitched enhancers were ranked by their ChIP-Seq read signal of H3K27Ac, using ROSE (available on the World Wide Web at subdomain bitbucket.org/young_computation/rose) (J. Loven et al., Cell, 11 Apr. 2013, 153:320.

### Assignment of Interactions to Regulatory Elements

To identify the association of long-range chromatin interactions to different regulatory elements, the PET peaks of interactions to different regulatory elements, including active enhancers (H3K27Ac binding sites), promoters (+/−2 kb of the Refseq TSS), and CTCF binding sites were assigned. For the analysis displayed on FIG. 17D, if an anchor site overlapped with multiple regulatory elements priority was assigned as: (1) promoters, (2) enhancers, (3) CTCF. A minimum of 1 base-pair overlap was required. These anchor classifications represent the nodes in FIG. 17D. Next, the edges were calculated by counting the number of interactions between the classified PET anchors. This analysis did not include CTCF sites that overlap either enhancers or promoters in the “CTCF” node of the plot. The total number of CTCF-CTCF interactions displayed on FIG. 7D includes interaction between any two CTCF-bound sites, regardless whether they overlap enhancers or promoters or not.

### Assignment of Genes to Enhancers

Enhancers were assigned to promoters by two measures. The enhancer-promoter ChIA-PET interactions were used to assign enhancers to their target genes. In the absence of a ChIA-PET interaction, enhancers were assigned to the nearest active gene. Active genes for the assignment were defined using H3K27Ac ChIP-Seq read densities around the transcription start site of the respective gene.

### Insulated Neighborhoods

Candidate insulated neighborhoods were defined as two CTCF sites that have an at least 1 bp overlap each with two PET peaks connected by a cohesin ChIA-PET interaction. A gene was considered to be inside an insulated neighborhood, if its transcription start site (TSS) is located within the neighborhood boundaries. In case multiple TSSs are annotated for the same gene, the TSS of the longest transcript was used for further analysis.

### Heatmap Representation of ChIA-PET Interactions in Insulated Neighborhoods

Heatmap representations of ChIA-PET interactions on FIG. 7E were created by mapping high-confidence ChIA-PET interactions across insulated neighborhoods using a previously described method. Three types of regions: upstream, the insulated neighborhood, and downstream were created. Upstream and downstream regions are 20% of the insulated neighborhood's length each. The upstream and downstream regions were divided into 10 equally sized bins each, and insulated neighborhoods were length normalized by dividing them into 50 equally-sized bins. To calculate interactions in each bin the interactions were filtered in two ways: (1) interactions were required to have at least one end in the interrogated region. This removed interactions that are anchored outside of our region of interest. (2) interactions that represent nested interactions (i.e. where one CTCF anchor site of two interactions are identical) were removed. The density of the whole spans of ChIA-PET interactions in each bin was next calculated in the units of number of interactions per bin. The density of ChIA-PET interactions was row-normalized to the row maximum for each domain.

### RNA Isolation and RNA-Seq

Jurkat RNA was isolated and sequences exactly as previously described (J. Loven et al., Cell, 26 Oct. 2012, 151:476). RNA-Seq reads were aligned to the hg19 (GRCh37) reference genome using Tophat2 (C. Trapnell, et al., Bioinformatics, 1 May 2009, 25:1105) version 2.0.11, using Bowtie version 2.2.1.0 and Samtools version 0.1.19.0. RPKMs per Refseq transcript were calculated from aligned reads using RPKM_count.py from RSeQC (50). For a gene to be considered expressed, the cutoff of >1 RPKM was used. For the analysis displayed on FIG. 8A, if multiple TSSs were annotated for the same gene, the RPKM value longest transcript was considered (this method of collapsing TSSs produced qualitatively identical results compared to using the RPKM value of the highest-expressed transcript).

### CRISPR/Cas9 Mediated Genome Editing

Genome editing was performed using CRISPR/Cas9. Target-specific oligonucleotides were cloned into a plasmid carrying a codon-optimized version of Cas9 and either an mCherry or GFP expression cassette (R. Jaenisch). SgRNA sequences were cloned into the BbsI recognition sites as described on the World Wide Web at subdomain genome-engineering.org/crispr/. The genomic sequences complementary to guide RNAs are listed below. HEK-293T cells were transfected with two plasmids expressing Cas9 and sgRNA targeting regions around 200 basepairs up- and down-stream of the center of the targeted CTCF site at the TAL1 locus, and 200 basepairs up- and down-stream of the first and third CTCF binding sites at the LMO2 locus, respectively. One of the two guide RNAs were cloned into the Cas9 expression vector containing the mCherry and the other into the Cas9 expression vector containing the GFP expression cassette. Transfection was carried out with the Lipofectamine 2000 reagent (Invitrogen Life Technologies, Grand Island, N.Y., US) according to the manufacturer's instructions. For the LMO2 locus 1 μl of a 10 μM repair template (160 bp ultramer with the desired deletion junction) was included in the transfection. Two days after transfections, cells positive for mCherry and GFP were FACS sorted, and replated at clonal density. Individual colonies were picked, expanded, and genotyped by PCR, and the edited alleles were verified by Sanger sequencing. The cell lines used for the expression analysis on FIG. 9 that carry a deletion allele at the TAL1 locus are homozygous, and the cell lines that carry a deletion allele at the D1402 locus are heterozygous for the modification.

### Sg1_TAL1: ACATTTCAATTATATGTTAA (SEQ ID NO: 1)

### Sg2_TALL ATACTAGTTAAGCTTTTCCT (SEQ ID NO: 2)

### Sg1_LMO2: AAACCAGCATTGCCACCTGG (SEQ ID NO: 3)

### Sg2_LMO2: CCAGGTGGCAATGCTGGTTT (SEQ ID NO: 4)

### LMO2 Repair Template: AGC CCC ATA GTT GGT GCT CAA TAA ATG CTA GTA ATA

### TTT ACT TGT GGC TTA CTG GTT CCT CAA GAT TCC TTA AAA TCT GAT GGC

### ATC AGA AGA GAC TAT CTC ACT GTT ATC ATG ACA TGG ACA TCC CGT GCA

### TGC CTG TAT TTG AAC ACT TGT CTC ATT G (SEQ ID NO: 5)

### RNA Isolation and Quantitative RT-PCR

RNA was isolated using the RNeasy purification kit (Promega Life Sciences (North America) Madison, Wis., US) and reverse transcribed using oligo-dT primers and SuperScript III reverse transcriptase (Invitrogen Life Technologies, Grand Island, N.Y., US) according to the manufacturers' instructions. Quantitative real-time PCR was performed on a 7000 AB Detection System using the following Taqman probes, according to the manufacturer's instructions (Applied Biosystems, Grand Island, N.Y., US):

GAPDH: hs02758991_g1

TALL hs01097987_m1

LMO2: hs00277106_m1

T-ALL deletion catalog and overlap analysis

Deletions in T-ALL genomes were compiled from multiple studies (L. Holmfeldt et al., Nature Genet, March 2013, 45:242; C. G. Mullighan et al., Nature, 12 Apr. 2007, 446:758; E. Papaemmanuil et al., Nature Genet, February 2014, 46:116; and J. Zhang et al., Nature, 12 Jan. 2012, 481:157). Short deletions (<500 kb, around half the size of an average TAD (16)) were filtered out in order to minimize deletions that affect multiple genes. The overlap with insulated neighborhood boundaries were done as follows:

A neighborhood boundary CTCF site was scored as overlapping a deletion, if the boundary site (i.e. the PET peak) overlapped at least one deletion by 1 bp. A deletion was scored as overlapping a neighborhood (i.e. the PET peak) boundary if it overlapped a boundary site by at least 1 bp. The deletion co-ordinates (hg19/GRCh37) and the source study are listed in Table S5.

### COSMIC Deletion Catalog and Overlap Analysis

Structural genomic rearrangements in cancer genomes were downloaded from the COSMIC database from the World Wide Web at subdomain cancer.sanger.ac.uk/cosmic using the hg19/GRCh37 genome assembly co-ordinates. Relatively short deletions (<500 kb, around half the size of an average TAD (16)) were filtered out in order to minimize deletions that affect multiple genes. The deletions annotated on “chr23” and “chr24” were removed for further analysis. The overlap with insulated neighborhood boundaries were done as follows. A neighborhood boundary site (i.e. the PET peak) was scored as overlapping a deletion, if it overlapped at least one deletion by 1 bp. A deletion was scored as overlapping a neighborhood boundary if it overlapped a boundary (i.e. the PET peak) by at least 1 bp.

### T-ALL Pathogenesis Genes

To identify a set of genes whose mutations have been causally linked to T-ALL, a list of genes was curated using the Cancer Gene Census and individual studies. The Cancer Gene Census was downloaded from the World Wide Web at subdomain cancer.sanger.ac.uk/cosmic. The complete Gene Census was filtered for genes that had “T-ALL” annotated in the “Tumor type” columns of the Gene Census. Genes that were described as recurrently altered in T-ALL in P. Van Vlierberghe and A. Ferrando, J Clin Invest, October 2012, 122:3398 were added. Gene symbols were converted into Refseq IDs for further analysis using the table described in the RNA-seq section. This resulted in a manually curated list of 55 genes (Table S3).

### Constitutive Interactions Across Three Cell Types

First, CTCF binding sites, cohesin binding sites were identified in Jurkat, GM12878 and K562 cells. Cohesin ChIA-PET in the three cell types were processed as described above, and two CTCF bound sites that are connected by a cohesin ChIA-PET interaction were annotated as CTCF-CTCF/cohesin interactions in each cell type (i.e. candidate insulated neighborhoods). For the overlap analysis displayed on FIGS. 21A-21B, binding peaks in the respective datasets were considered shared is they overlapped by at least 1 bp. On FIG. 21C, the CTCF-CTCF/cohesin interactions were scored as constitutive across two cell types if they had a reciprocal overlap of at least 95% of the length of the interaction. The ChIA-PET datasets are not saturated, suggesting that not every interaction found within a cell will be potentially represented in the dataset. Therefore, candidate constitutive CTCF-CTCF/cohesin interactions were defined as the set of CTCF-CTCF/cohesin interactions that were found overlapping in at least two of the three cell types. This resulted in 13,908 constitutive CTCF-CTCF loops (Table S6).

### Definition of Proto-Oncogene Neighborhoods

Candidate proto-oncogenes were identified as follows. The genes listed in the Cancer Gene Census were downloaded from the World Wide Web at subdomain cancer.sanger.ac.ukkosmic, a list that contains the genes whose mutations have been causally linked to cancer (i.e. both candidate proto-oncogenes and tumor suppressor genes). Proto-oncogenes are generally activated by mutations that result in a dominant phenotype and tumor suppressor genes are de-activated by mutations that have a recessive phenotype, so the genes whose mutations are annotated as dominant in the Cancer Gene Census were filtered. This resulted in 329 candidate proto-oncogenes.

Proto-oncogene neighborhoods were subsequently defined as a constitutive CTCF-CTCF interaction (i.e. an interaction found in at least two of three cell types; see above) that encompassed the transcription start site (TSS) of at least one gene of the 329 candidate proto-oncogenes. When multiple CTCF-CTCF loops encompassed the TSS of a candidate proto-oncogene, only the shortest one was considered for further analysis. These are listed in Table S8.

For the example proto-oncogenes whose neighborhood is disrupted by a deletion (FIG. 10C) the following studies demonstrate activation of the genes in the cancer types the deletion was documented in:
FGFR1, colorectal cancer: J. H. Jang, Oncogene, 27 Jan. 2005, 24:945
EGFR1, pancreatic cancer: J. Dancer et al., Oncol Rep, July 2007, 18:151
MAP2K2, pancreatic cancer: X. Tan et al., Internat'l J Oncol, January 2004, 24:65
CCND1, pancreatic cancer: M. M. Al-Aynati et al., Clin Cancer Res, 1 Oct. 2004, 10:6598
HMGA1, ovarian cancer: S. Camilleri-Broet et al., Annals Oncol, January 2004, 15:104
REL, leukemia: S. Hartmann et al., Brit J Haematol, February 2010, 148:402

### Accession Numbers

Data generated in this study have been deposited in the Gene Expression Omnibus under the Accession Numbers GSE68978 and are incorporated herein by reference. The GEO Accession numbers of the datasets used in this example are listed in Table S11.

### Example 3: Disruption of Insulated Neighborhood Boundaries is Linked to Proto-Oncogene Activation

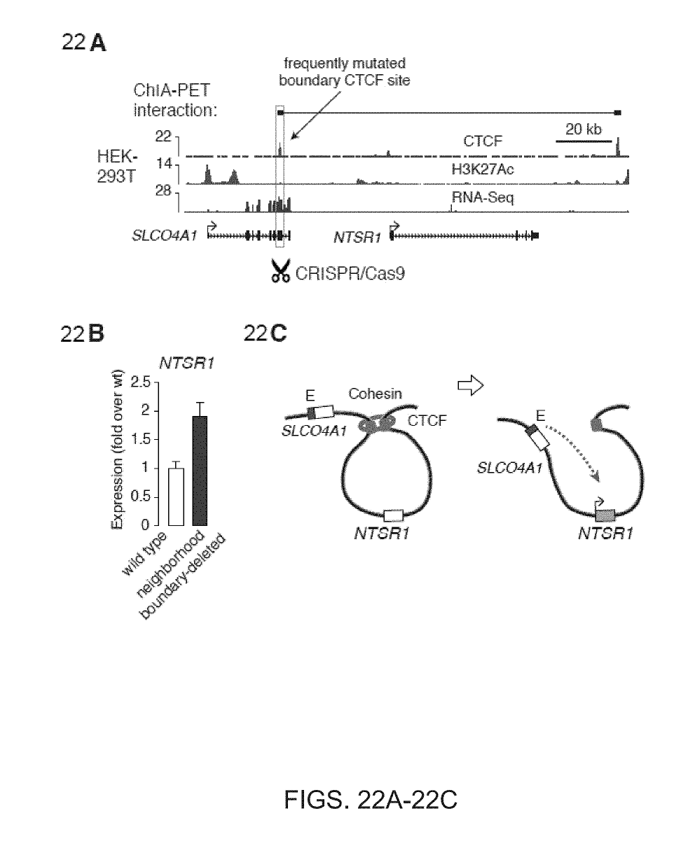

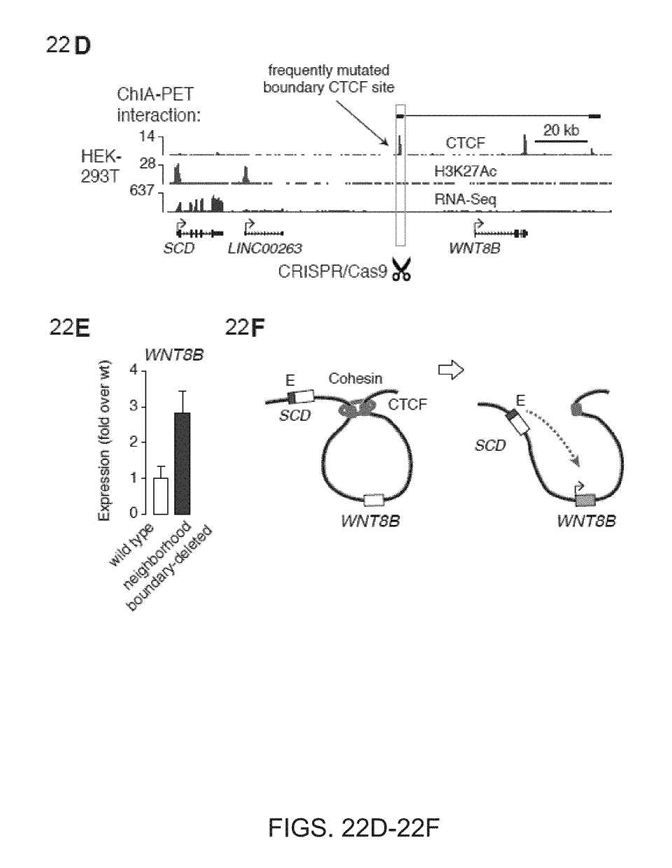

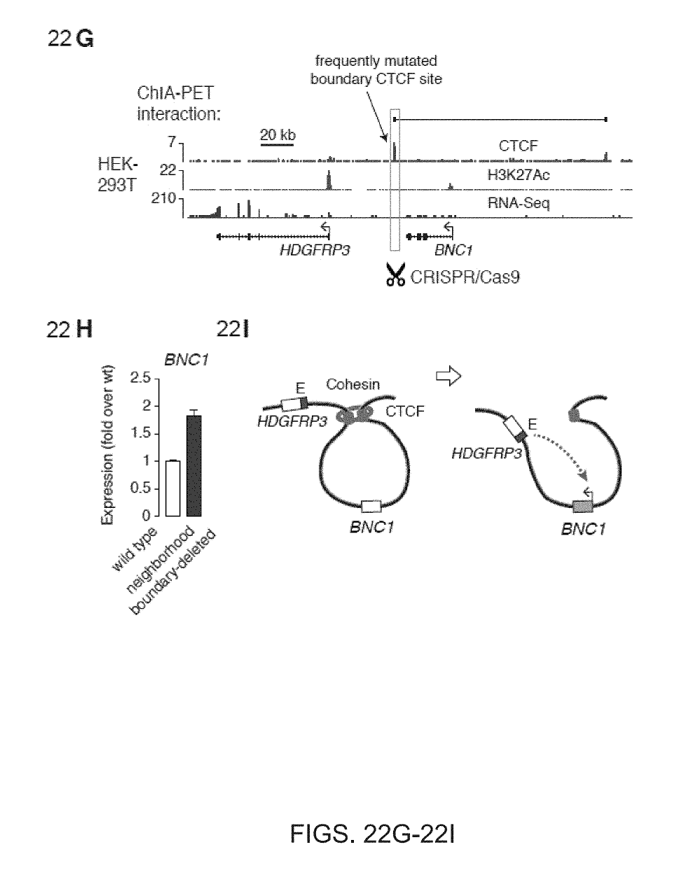

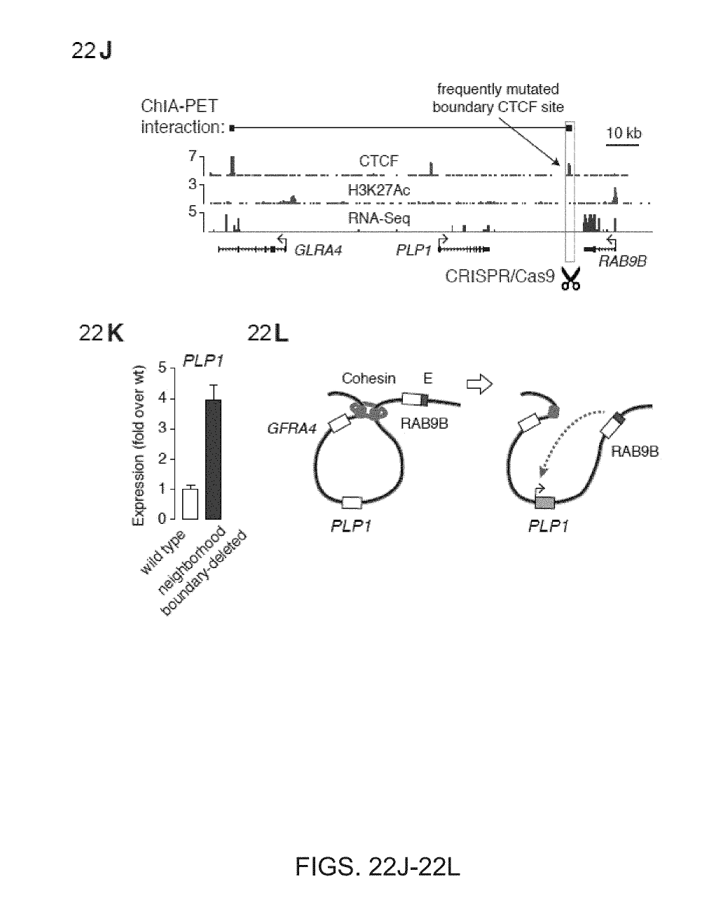

Upon a search of the International Cancer Genome Consortium (ICGC) database and individual cancer genome studies (Katainen et al, 2015), proto-oncogenes located within insulated neighborhoods having boundaries that were frequently mutated in cancer were identified. These genes included NTSR1 (FIGS. 22A-22C), WNT8B (FIGS. 22D-22F), BNC1 (FIGS. 22G-22I) and PLY 1 (FIGS. 22J-22L). The CTCF boundary site located near each examined gene was then deleted in HEK-293T cells using a CRISPR/Cas9-based approach (FIGS. 22A, 22D, 22G and 22J). Using qRT-PCR, expression of the genes in wild type HEK-293T cells was then measured and compared to expression of the genes in HEK-293T cells where the neighborhood boundary was deleted. The PCR analysis showed that, in each case, expression of the genes was greater in cells where the boundary was deleted (FIGS. 22B, 22E, 22H and 22K). It is expected that the increase in expression occurs because deletion of the CTCF boundary site brought each gene under the regulatory influence of an enhancer of a nearby gene located outside the insulated neighborhood (FIGS. 22C, 22F, 22I and 22K), resulting in transcriptional activation.

### Definitions

FGFR1 fibroblast growth factor receptor 1
EGFR1 epidermal growth factor receptor 1
MAP2K mitogen-activated protein kinase 2
CCND1 cyclin D1
HMGA1 high-mobility group AT-HOOK 1
RECA recombination protein A

### OLIG2

SMC1 structural maintenance of chromosomes 1A

### MYB V-Myb Avian Myeloblastosis Viral Oncogene Homolog

### RUNX1 Runt-related Transcription Factor 1

### GATA3 GATA Binding Protein 3

### TAL1 T-cell Acute Lymphocytic Leukemia 1

### POLR2A Polymerase (RNA) II (DNA Directed) Polypeptide A

### RNAPII RNA Polymerase II

### H3K27Ac

### CTCF CCCTC-binding Factor

### LMO1 LIM Domain Only 1

### TLX1 T-cell Leukemia Homeobox 1

### OLIG2 Oligodendrocyte Lineage Transcription Factor 2

### TLX3 T-cell Leukemia Homeobox 3

### RAD21 RAD21 Homolog

NSCLC non-small cell lung cancer
CML chronic myelogenous leukemia
AML acute myeloid leukemia
T-All T-cell acute lymphoblastic leukemia
GBM glioblastoma multiforme
SCLC small cell lung cancer

### REFERENCES

Banerji, J., Rusconi, S., and Schaffner, W. (1981). Expression of a beta-globin gene is enhanced by remote SV40 DNA-sequences. Cell 27, 299-308.
Bell, A. C., West, A. G., and Felsenfeld, G. (1999). The protein CTCF is required for the enhancer blocking activity of vertebrate insulators. Cell 98, 387-396.
Benjamini, Y., and Hochberg, Y. (1995). Controlling the false discovery rate: a practical and powerful approach to multiple testing. Journal of the Royal Statistical Society Series B-Methodological 57, 289-300.
Benoist, C., and Chambon, P. (1981). In vivo sequence requirements of the SV40 early promoter region. Nature 290, 304-310.
Bickmore, W. A. (2013). The Spatial Organization of the Human Genome. Annual Review of Genomics and Human Genetics, Vol 14 14, 67-84.
Bilodeau, S., Kagey, M. H., Frampton, G. M., Rahl, P. B., and Young, R. A. (2009). SetDB1 contributes to repression of genes encoding developmental regulators and maintenance of ES cell state. Genes Dev 23, 2484-2489.
Cai, H., and Levine, V. (1995). Modulation of enhancer-promoter interactions by insulators in the drosophila embryo. Nature 376, 533-536.
Chan, Y.-S., Goeke, J., Ng, J.-H., Lu, X., Gonzales, K. A. U., Tan, C.-P., Tng, W.-Q., Hong, Z.-Z., Lim, Y.-S., and Ng, H.-H. (2013). Induction of a Human Pluripotent State with Distinct Regulatory Circuitry that Resembles Preimplantation Epiblast. Cell Stem Cell 13, 663-675.
Chen, H., Tian, Y., Shu, W., Bo, X., and Wang, S. (2012). Comprehensive identification and annotation of cell type-specific and ubiquitous CTCF-binding sites in the human genome. PLoS One 7, e41374.
Cheng, Y., Ma, Z., Kim, B.-H., Wu, W., Cayting, P., Boyle, A. P., Sundaram, V., Xing, X., Dogan, N., Li, J., et al. (2014). Principles of regulatory information conservation between mouse and human. Nature 515, 371-375.
Chia, N.-Y., Chan, Y.-S., Feng, B., Lu, X., Orlov, Y. L., Moreau, D., Kumar, P., Yang, L., Jiang, J., Lau, M.-S., et al. (2010). A genome-wide RNAi screen reveals determinants of human embryonic stem cell identity. Nature 468, 316-320.
Cuddapah, S., Jothi, R., Schones, D. E., Rob, T. Y., Cui, K., and Zhao, K. (2009). Global analysis of the insulator binding protein CTCF in chromatin barrier regions reveals demarcation of active and repressive domains. Genome Res 19, 24-32.
de Graaf, C. A., and van Steensel, B. (2013). Chromatin organization: form to function. Current Opinion in Genetics & Development 23, 185-190.
de Laat, W., and Duboule, D. (2013). Topology of mammalian developmental enhancers and their regulatory landscapes. Nature 502, 499-506.
De Los Angeles, A., Loh, Y. H., Tesar, P. J., and Daley, G. Q. (2012). Accessing naive human pluripotcncy. Curr Opin Genet Dev 22, 272-282.
Dekker, J. (2014). Two ways to fold the genome during the cell cycle: insights obtained with chromosome conformation capture. Epigenetics & chromatin 7, 25.
DeMare, L. E., Leng, J., Cotney, J., Reilly, S. K., Yin, J., Sarro, R., and Noonan, J. P. (2013). The genomic landscape of cohesin-associated chromatin interactions. Genome Res 23, 1224-1234.
Ding, L., Paszkowski-Rogacz, M., Nitzsche, A., Slabicki, M. M., Heninger, A. K., de Vries, 1., Kittler, R., Junqueira, M., Shevchenko, A., Schulz, H., et al. (2009). A genome-scale RNAi screen for Oct4 modulators defines a role of the Paf1 complex for embryonic stem cell identity. Cell Stem Cell 4, 403-415.
Dixon, J. R., Jung, I., Selvaraj, S., Shen, Y., Antosiewicz-Bourget, J. E., Lee, A. Y., Ye, Z., Kim, A., Rajagopal, N., Xie, W., et al. (2015). Chromatin architecture reorganization during stem cell differentiation. Nature 518, 331-336.
Dixon, J. R., Selvaraj, S., Yue, F., Kim, A., Li, Y., Shen, Y., Hu, M., Liu, J. S., and Ren, B. (2012). Topological domains in mammalian genomes identified by analysis of chromatin interactions. Nature 485, 376-380.
Dorsett, D., and Merkenschlager, M. (2013). Cohesin at active genes: a unifying theme for cohesin and gene expression from model organisms to humans. Curr Opin Cell Biol 25, 327-333.
Dowen, J. M., Bilodeau, S., Orlando, D. A., Hubner, M. R., Abraham, B. J., Spector, D. L., and Young, R. A. (2013). Multiple structural maintenance of chromosome complexes at transcriptional regulatory elements. Stem cell reports 1, 371-378.
Dowen, J. M., Fan, Z. P., Hnisz, D., Ren, G., Abraham, B. J., Zhang, L. N., Weintraub, A. S., Schuijers, J., Lee, T. I., Zhao, K., et al. (2014). Control of Cell Identity Genes Occurs in Insulated Neighborhoods in Mammalian Chromosomes. Cell 159, 374-387.
Dunham, I., Kundaje, A., Aldred, S. F., Collins, P. J., Davis, C., Doyle, F., Epstein, C. B., Frietze, S., Harrow, J., Kaul, R., et al. (2012). An integrated encyclopedia of DNA elements in the human genome. Nature 489, 57-74.
Fazzio, T. G., Huff, J. T., and Panning, B. (2008). An RNAi screen of chromatin proteins identifies Tip60-p400 as a regulator of embryonic stem cell identity. Cell 134, 162-174.
Filippova, G. N., Fagerlie, S., Klenova, E. M., Myers, C., Dehner, Y., Goodwin, G., Neiman, P. E., Collins, S. J., and Lobanenkov, V. V. (1996). An exceptionally conserved transcriptional repressor, CTCF, employs different combinations of zinc fingers to bind diverged promoter sequences of avian and mammalian c-myc oncogenes. Molecular and Cellular Biology 16, 2802-2813.
Fullwood, M. J., Liu, M. H., Pan, Y. F., Liu, J., Xu, H., Mohamed, Y. B., Orlov, Y. L., Velkov, S., Ho, A., Mei, P. H., et al. (2009). An oestrogen-receptor-alpha-bound human chromatin interactome. Nature 462, 58-64.
Gafni, O., Weinberger, L., Mansour, A. A., Manor, Y. S., Chomsky, E., Ben-Yosef, D., Kalma, Y., Viukov, S., Maza, I., Zviran, A., et al. (2013). Derivation of novel human ground state naive pluripotent stem cells. Nature 504, 282-286.
Geyer, P. K., and Corces, V. G. (1992). DNA position-specific repression of transcription by a drosophila zinc finger protein. Genes & Development 6, 1865-1873.
Gibcus, J. H., and Dekker, J. (2012). The context of gene expression regulation. F1000 biology reports 4, 8.
Giorgio, E., Robyr, D., Spiclmann, M., Ferrero, E., Di Gregorio, E., Imperiale, D., Vaula, G., Stamoulis, G., Santoni, F., Atzori, C., et al. (2015). A large genomic deletion leads to enhancer adoption by the lamin B1 gene: a second path to autosomal dominant adult-onset demyelinating leukodystrophy (ADLD). Human molecular genetics 24, 3143-3154.
Gomez-Diaz, E., and Corces, V. G. (2014). Architectural proteins: regulators of 3D genome organization in cell fate. Trends in Cell Biology 24, 703-711.
Gorkin, D. U., Leung, D., and Ren, B. (2014). The 3D Genome in Transcriptional Regulation and Pluripotency. Cell Stem Cell 14, 762-775.
Grant, C. E., Bailey, T. L., and Noble, W. S. (2011). FIMO: scanning for occurrences of a given motif. Bioinformatics 27, 1017-1018.
Gruss, P., Dhar, R., and Khoury, G. (1981). Simian virus-40 tandem repeated sequences as an element of the early promoter. Proceedings of the National Academy of Sciences of the United States of America-Biological Sciences 78, 943-947.
Hackett, J. A., and Surani, M. A. (2014). Regulatory principles of pluripotency: from the ground state up. Cell Stem Cell 15, 416-430.
Hadjur, S., Williams, L. M., Ryan, N. K., Cobb, B. S., Sexton, T., Fraser, P., Fisher, A. G., and Merkenschlager, M. (2009). Cohesins form chromosomal cis-interactions at the developmentally regulated IFNG locus. Nature 460, 410-413.
Hanahan, D., and Weinberg, R. A. (2011). Hallmarks of cancer: the next generation. Cell 144, 646-674.
Handoko, L., Xu, H., Li, G., Ngan, C. Y., Chew, E., Schnapp, M., Lee, C. W., Ye, C., Ping, J. L., Mulawadi, F., et al. (2011). CTCF-mediated functional chromatin interactome in pluripotent cells. Nat Genet 43, 630-638.
Heidari N, P. D., He C, Grubert F, Jahanbanian F, Kasowski M, Zhang M Q, Snyder M P. (2014). Genome-wide map of regulatory interactions in the human genome. Genome Res 24, 1905-1917.
Heidari, N., Phanstiel, D. H., He, C., Grubert, F., Jahanbani, F., Kasowski, M., Zhang, M. Q., and Snyder, M. P. (2014). Genome-wide map of regulatory interactions in the human genome. Genome Res 24, 1905-1917.
Heinz, S., Romanoski, C. E., Benner, C., and Glass, C. K. (2015). The selection and function of cell type-specific enhancers. Nature reviews Molecular cell biology 16, 144-154.
Hnisz, D., Abraham, B. J., Lee, T. I., Lau, A., Saint-Andre, V., Sigova, A. A., Hoke, H. A., and Young, R. A. (2013). Super-Enhancers in the Control of Cell Identity and Disease. Cell 155, 934-947.
Hu, G., Kim, J., Xu, Q., Leng, Y., Orkin, S. H., and Elledge, S. J. (2009). A genome-wide RNAi screen identifies a new transcriptional module required for self-renewal. Genes Dev 23, 837-848.
Ibn-Salem, J., Kohler, S., Love, M. I., Chung, H. R., Huang, N., Hurles, M. E., Haendel, M., Washington, N. L., Smedley, D., Mungall, C. J., et al. (2014). Deletions of chromosomal regulatory boundaries are associated with congenital disease. Genome Biol 15, 423.
Imakaev, M., Fudenberg, G., McCord, R. P., Naumova, N., Goloborodko, A., Lajoie, B. R., Dekker, J., and Mirny, L. A. (2012). Iterative correction of Hi-C data reveals hallmarks of chromosome organization. Nature Methods 9, 999-1003.
Jeppsson, K., Kanno, T., Shirahige, K., and Sjogren, C. (2014). The maintenance of chromosome structure: positioning and functioning of SMC complexes. Nature reviews Molecular cell biology 15, 601-614.
Ji, X., Dadon, D. B., Abraham, B. J., Lee, T. I., Jaenisch, R., Bradner, J. E., and Young, R. A. (2015). Chromatin proteomic profiling reveals novel proteins associated with histone-marked genomic regions. Proceedings of the National Academy of Sciences of the United States of America 112, 3841-3846.
Kagey, M. H., Newman, J. J., Bilodeau, S., Zhan, Y., Orlando, D. A., van Berkum, N. L., Ebmcicr, C. C., Goossens, J., Rahl, P. B., Levine, S. S., et al. (2010). Mediator and cohcsin connect gene expression and chromatin architecture. Nature 467, 430-435.
Katainen, R., Dave, K., Pitkanen, E., Palin, K., Kivioja, T., Valimaki, N., Gylfe, A. E., Ristolainen, H., Hanninen, U. A., Cajuso, T., et al. (2015). CTCF/cohesin-binding sites are frequently mutated in cancer. Nat Genet 2015 July; 47(7):818-21. doi: 10.1038/ng.3335. Epub 2015 Jun. 8.
Kellis, M., Wold, B., Snyder, M. P., Bernstein, B. E., Kundaje, A., Marinov, G. K., Ward, L. D., Birney, E., Crawford, G. E., Dekker, J., et al. (2014). Defining functional DNA elements in the human genome. Proc Natl Acad Sci USA 111, 6131-6138.
Kent, W. J., Sugnet, C. W., Furey, T. S., Roskin, K. M., Pringle, T. H., Zahler, A. M., and Haussler, D. (2002). The human genome browser at UCSC. Genome Research 12, 996-1006.
Kheradpour, P., Ernst, J., Melnikov, A., Rogov, P., Wang, L., Zhang, X., Alston, J., Mikkelsen, T. S., and Kellis, M. (2013). Systematic dissection of regulatory motifs in 2000 predicted human enhancers using a massively parallel reporter assay. Genome Res 23, 800-811.
Kieffer-Kwon, K.-R., Tang, Z., Mathe, E., Qian, J., Sung, M.-H., Li, G., Resch, W., Baek, S., Pruett, N., Grontved, L., et al. (2013). Interactome Maps of Mouse Gene Regulatory Domains Reveal Basic Principles of Transcriptional Regulation. Cell 155, 1507-1520.
Kim, T. H., Abdullaev, Z. K., Smith, A. D., Ching, K. A., Loukinov, D. I., Green, R. D., Zhang, M. Q., Lobanenkov, V. V., and Ren, B. (2007). Analysis of the vertebrate insulator protein CTCF-binding sites in the human genome. Cell 128, 1231-1245.
Kundaje, A., Meuleman, W., Ernst, J., Bilenky, M., Yen, A., Heravi-Moussavi, A., Kheradpour, P., Zhang, Z., Wang, J., Ziller, M. J., et al. (2015). Integrative analysis of 111 reference human epigenomes. Nature 518, 317-330.
Langmead, B., Trapnell, C., Pop, M., and Salzberg, S. L. (2009). Ultrafast and memory-efficient alignment of short DNA sequences to the human genome. Genome Biology 10.
Lee, T. I., and Young, R. A. (2013). Transcriptional Regulation and Its Misregulation in Disease. Cell 152, 1237-1251.
Leung, D., Jung, 1., Rajagopal, N., Schmitt, A., Selvaraj, S., Lee, A. Y., Yen, C. A., Lin, S., Lin, Y., Qiu, Y., et al. (2015). Integrative analysis of haplotype-resolved epigenomes across human tissues. Nature 518, 350-354.
Levine, M., Cattoglio, C., and Tjian, R. (2014). Looping Back to Leap Forward: Transcription Enters a New Era. Cell 157, 13-25.
Li, G., Ruan, X., Auerbach, R. K., Sandhu, K. S., Zheng, M., Wang, P., Poh, H. M., Goh, Y., Lim, J., Zhang, J., et al. (2012). Extensive Promoter-Centered Chromatin Interactions Provide a Topological Basis for Transcription Regulation. Cell 148, 84-98.
Li, G., Fullwood, M. J., Xu, H., Mulawadi, F. H., Velkov, S., Vega, V., Ariyaratne, P. N., Bin Mohamed, Y., Ooi, H.-S., Tennakoon, C., et al. (2010). ChIA-PET tool for comprehensive chromatin interaction analysis with paired-end tag sequencing. Genome Biology 11.
Lieberman-Aiden, E., van Berkum, N. L., Williams, L., Imakaev, M., Ragoczy, T., Telling, A., Amit, I., Lajoic, B. R., Sabo, P. J., Dorschner, M. O., et al. (2009). Comprehensive Mapping of Long-Range Interactions Reveals Folding Principles of the Human Genome. Science 326, 289-293.
Loven, J., Hoke, H. A., Lin, C. Y., Lau, A., Orlando, D. A., Vakoc, C. R., Bradner, J. E., Lee, T. I., and Young, R. A. (2013). Selective inhibition of tumor oncogenes by disruption of super-enhancers. Cell 153, 320-334.
Lupianez, D. G., Kraft, K., Heinrich, V., Krawitz, P., Brancati, F., Klopocki, E., Horn, D., Kayserili, H., Opitz, J. M., Laxova, R., et al. (2015). Disruptions of topological chromatin domains cause pathogenic rewiring of gene-enhancer interactions. Cell 161, 1012-1025.
Maejima, T., Inoue, T., Kanki, Y., Kohro, T., Li, G., Ohta, Y., Kimura, H., Kobayashi, M., Taguchi, A., Tsutsumi, S., et al. (2014). Direct evidence for pitavastatin induced chromatin structure change in the KLF4 gene in endothelial cells. PLoS One 9, e96005.
Martello, G., and Smith, A. (2014). The nature of embryonic stem cells. Annual review of cell and developmental biology 30, 647-675.
Martin, M. (2011). Cutadapt removes adapter sequences from high-throughput sequencing reads. EMBnet journal 17, 1-10.
Matys, V., Kel-Margoulis, O. V., Fricke, E., Liebich, I., Land, S., Barre-Dirrie, A., Reuter, I., Chekmenev, D., Krull, M., Hornischer, K., et al. (2006). TRANSFAC (R) and its module TRANSCompel (R): transcriptional gene regulation in eukaryotes. Nucleic Acids Research 34, D108-110.
Merkenschlager, M., and Odom, D. T. (2013). CTCF and Cohesin: Linking Gene Regulatory Elements with Their Targets. Cell 152, 1285-1297.
Mizuguchi, T., Fudenberg, G., Mehta, S., Belton, J.-M., Taneja, N., Folco, H. D., FitzGerald, P., Dekker, J., Mirny, L., Barrowman, J., et al. (2014). Cohesin-dependent globules and heterochromatin shape 3D genome architecture in S. pombe. Nature 516, 432-435.
Narendra, V., Rocha, P. P., An, D., Raviram, R., Skok, J. A., Mazzoni, E. O., and Reinberg, D. (2015). Transcription. CTCF establishes discrete functional chromatin domains at the Hox clusters during differentiation. Science 347, 1017-1021.
Nativio, R., Wendt, K. S., Ito, Y., Huddleston, J. E., Uribe-Lewis, S., Woodfine, K., Krueger, C., Reik, W., Peters, J. M., and Murrell, A. (2009). Cohesin is required for higher-order chromatin conformation at the imprinted IGF2-H19 locus. PLoS Genet 5, e1000739.
Nora, E. P., Lajoie, B. R., Schulz, E. G., Giorgetti, L., Okamoto, 1., Servant, N., Piolot, T., van Berkum, N. L., Meisig, J., Sedat, J., et al. (2012). Spatial partitioning of the regulatory landscape of the X-inactivation centre. Nature 485, 381-385.
Parelho, V., Hadjur, S., Spivakov, M., Leleu, M., Sauer, S., Gregson, H. C., Jarmuz, A., Canzonetta, C., Webster, Z., Nesterova, T., et al. (2008). Cohesins functionally associate with CTCF on mammalian chromosome arms. Cell 132, 422-433.
Phillips-Cremins, J. E., Sauria, M. E. G., Sanyal, A., Gerasimova, T. I., Lajoie, B. R., Bell, J. S. K., Ong, C.-T., Hookway, T. A., Guo, C., Sun, Y., et al. (2013). Architectural Protein Subclasses Shape 3D Organization of Genomes during Lineage Commitment. Cell 153, 1281-1295.
Plank, J. L., and Dean, A. (2014). Enhancer function: mechanistic and genome-wide insights come together. Mol Cell 55, 5-14.
Pollard, K. S., Hubisz, M. J., Rosenbloom, K. R., and Siepel, A. (2010). Detection of nonneutral substitution rates on mammalian phylogenies. Genome Research 20, 110-121.
Pombo, A., and Dillon, N. (2015). Three-dimensional genome architecture: players and mechanisms. Nature reviews Molecular cell biology 16, 245-257.
Pope, B. D., Ryba, T., Dileep, V., Yue, F., Wu, W., Denas, O., Vera, D. L., Wang, Y., Hansen, R. S., Canfield, T. K., et al. (2014). Topologically associating domains are stable units of replication-timing regulation. Nature 515, 402-405.
Rao, S. S. P., Huntley, M. H., Durand, N. C., Stamenova, E. K., Bochkov, I. D., Robinson, J. T., Sanborn, A. L., Machol, I., Omer, A. D., Lander, E. S., et al. (2014). A 3D Map of the Human Genome at Kilobase Resolution Reveals Principles of Chromatin Looping. Cell 159, 1665-1680.
Remeseiro, S., and Losada, A. (2013). Cohesin, a chromatin engagement ring. Curr Opin Cell Biol 25, 63-71.
Robinson, M. D., McCarthy, D. J., and Smyth, G. K. (2010). edgeR: a Bioconductor package for differential expression analysis of digital gene expression data. Bioinformatics 26, 139-140.
Rubio, E. D., Reiss, D. J., Weicsh, P. L., Disteche, C. M., Filippova, G. N., Baliga, N. S., Aebersold, R., Ranish, J. A., and Krumm, A. (2008). CTCF physically links cohesin to chromatin. Proceedings of the National Academy of Sciences of the United States of America 105, 8309-8314.
Sanyal, A., Lajoie, B. R., Jain, G., and Dekker, J. (2012). The long-range interaction landscape of gene promoters. Nature 489, 109-113.
Schmidt, D., Schwalie, P. C., Wilson, M. D., Ballester, B., Goncalves, A., Kutter, C., Brown, G. D., Marshall, A., Flicek, P., and Odom, D. T. (2012). Waves of retrotransposon expansion remodel genome organization and CTCF binding in multiple mammalian lineages. Cell 148, 335-348.
Seitan, V. C., Faure, A. J., Zhan, Y., McCord, R. P., Lajoie, B. R., Ing-Simmons, E., Lenhard, B., Giorgetti, L., Heard, E., Fisher, A. G., et al. (2013). Cohesin-based chromatin interactions enable regulated gene expression within preexisting architectural compartments. Genome Research 23, 2066-2077.
Shlyueva, D., Stampfel, G., and Stark, A. (2014). Transcriptional enhancers: from properties to genome-wide predictions. Nature reviews Genetics 15, 272-286.
Smallwood, A., and Ren, B. (2013). Genome organization and long-range regulation of gene expression by enhancers. Curr Opin Cell Biol 25, 387-394.
Smith, E., and Shilatifard, A. (2014). Enhancer biology and enhanceropathies. Nat Struct Mol Biol 21, 210-219.
Sofueva, S., Yaffe, E., Chan, W. C., Georgopoulou, D., Vietri Rudan, M., Mira-Bontenbal, H., Pollard, S. M., Schroth, G. P., Tanay, A., and Hadjur, S. (2013). Cohesin-mediated interactions organize chromosomal domain architecture. The EMBO journal 32, 3119-3129.
Spitz, F., and Furlong, E. E. M. (2012). Transcription factors: from enhancer binding to developmental control. Nature Reviews Genetics 13, 613-626.
Takashima, Y., Guo, G., Loos, R., Nichols, J., Ficz, G., Krueger, F., Oxley, D., Santos, F., Clarke, J., Mansfield, W., et al. (2014). Resetting Transcription Factor Control Circuitry toward Ground-State Pluripotency in Human. Cell 158, 1254-1269.
Theunissen, T. W., Powell, B. E., Wang, H., Mitalipova, M., Faddah, D. A., Reddy, J., Fan, Z. P., Maetzel, D., Ganz, K., Shi, L., et al. (2014). Systematic Identification of Culture Conditions for Induction and Maintenance of Naive Human Pluripotency. Cell Stem Cell 15, 471-487.
Thurman, R. E., Rynes, E., Humbert, R., Vicrstra, J., Maurano, M. T., Haugen, E., Sheffield, N. C., Stergachis, A. B., Wang, H., Vernot, B., et al. (2012). The accessible chromatin landscape of the human genome. Nature 489, 75-82.
Trapnell, C., Pachter, L., and Salzberg, S. L. (2009). TopHat: discovering splice junctions with RNA-Seq. Bioinformatics 25, 1105-1111.
Trapnell, C., Williams, B. A., Pertea, G., Mortazavi, A., Kwan, G., van Baren, M. J., Salzberg, S. L., Wold, B. J., and Pachter, L. (2010). Transcript assembly and quantification by RNA-Seq reveals unannotated transcripts and isoform switching during cell differentiation. Nature Biotechnology 28, 511-515.
Vietri Rudan, M., Barrington, C., Henderson, S., Ernst, C., Odom, D. T., Tanay, A., and Hadjur, S. (2015). Comparative Hi-C reveals that CTCF underlies evolution of chromosomal domain architecture. Cell Rep 10, 1297-1309.
Wang, H., Maurano, M. T., Qu, H., Varley, K. E., Gertz, J., Pauli, F., Lee, K., Canfield, T., Weaver, M., Sandstrom, R., et al. (2012). Widespread plasticity in CTCF occupancy linked to DNA methylation. Genome Res 22, 1680-1688.
Ware, C. B., Nelson, A. M., Mecham, B., Hesson, J., Zhou, W., Jonlin, E. C., Jimenez-Caliani, A. J., Deng, X., Cavanaugh, C., Cook, S., et al. (2014). Derivation of naive human embryonic stem cells. Proc Natl Acad Sci USA 111, 4484-4489.
Welter, D., MacArthur, J., Morales, J., Burdett, T., Hall, P., Junkins, H., Klemm, A., Flicek, P., Manolio, T., Hindorff, L., et al. (2014). The NHGRI GWAS Catalog, a curated resource of SNP-trait associations. Nucleic Acids Research 42, D1001-1006.
Wendt, K. S., Yoshida, K., Itoh, T., Bando, M., Koch, B., Schirghuber, E., Tsutsumi, S., Nagae, G., Ishihara, K., Mishiro, T., et al. (2008). Cohesin mediates transcriptional insulation by CCCTC-binding factor. Nature 451, 796-801.
Whyte, W. A., Orlando, D. A., Hnisz, D., Abraham, B. J., Lin, C. Y., Kagey, M. H., Rahl, P. B., Lee, T. I., and Young, R. A. (2013). Master Transcription Factors and Mediator Establish Super-Enhancers at Key Cell Identity Genes. Cell 153, 307-319.
Zhang, J., Baran, J., Cros, A., Guberman, J. M., Haider, S., Hsu, J., Liang, Y., Rivkin, E., Wang, J., Whiny, B., et al. (2011). International Cancer Genome Consortium Data Portal-a one-stop shop for cancer genomics data. Database—the Journal of Biological Databases and Curation.
Zhang, Y., Liu, T., Meyer, C. A., Eeckhoute, J., Johnson, D. S., Bernstein, B. E., Nussbaum, C., Myers, R. M., Brown, M., Li, W., et al. (2008). Model-based Analysis of ChIP-Seq (MACS). Genome Biology 9.
Zuin, J., Dixon, J. R., van der Reijden, M. I. J. A., Ye, Z., Kolovos, P., Brouwer, R. W. W., van de Corput, M. P. C., van de Werken, H. J. G., Knoch, T. A., van Ijcken, W. F. J., et al. (2014). Cohesin and CTCF differentially affect chromatin architecture and gene expression in human cells. Proceedings of the National Academy of Sciences of the United States of America 111, 996-1001.

What is claimed is:

1. A method of identifying one or more differences in a regulatory pathway between two cells comprising obtaining expression data for at least one enhancer from each cell from an insulated neighborhood conserved between the two cells and comparing said expression data to identify differential activity of said enhancer on at least one target gene.

2. A method according to claim 1, wherein the cells are embryonic stem cells.

3. A method according to claim 1, wherein the cells are iPS cells.

4. A method according to claim 2, wherein one cell is naïve and one cell is primed.

5. A method according to claim 1, wherein one cell is a more differentiated cell type than the other cell.

6. A method for identifying a Topologically Associating Domain (TAD) comprising identifying TAD boundaries utilizing ChIA-PET data and identifying a TAD between two TAD boundaries.

7. A method according to claim 6, wherein the ChIA-PET data is cohesin ChIA-PET data.

8. A method according to claim 6, wherein the ChIA-PET data is processed using a Hidden Markov algorithm.

9. A method of inhibiting activation of a proto-oncogene by an enhancer, wherein one of the proto-oncogene or enhancer is located within an insulated neighborhood, comprising stabilizing the boundary of the insulated neighborhood such that disruption of the neighborhood is reduced, thereby inhibiting interaction of the enhancer with the proto-oncogene.

10. A method according to claim 9, wherein the proto-oncogene is located within the insulated neighborhood.

11. A method according to claim 9, wherein the enhancer is located within the insulated neighborhood.

12. A method according to claim 9, wherein the enhancer and the proto-oncogene are each located within an insulated neighborhood, and wherein the insulated neighborhoods are different from one another.

13. A method of identifying a super-enhancer in a 3D regulatory landscape of a cell comprising:
a. examining all enhancer activity within an insulated neighborhood; and
b. stitching all enhancers located within the insulated neighborhood together to form a super-enhancer.

14. The method of claim 13, wherein the super-enhancer is identified by performing chromatin immunoprecipitation high throughput sequencing (ChIP-Seq).

15. The method of claim 13, wherein the enhancers are located within a predetermined distance of each other.

16. The method of claim 15, wherein the enhancers are located within 12.5 kb of each other.

17. The method of claim 13, further comprising identifying a gene associated with the super-enhancer.

18. The method of claim 17, wherein the associated gene is identified by proximity to the super-enhancer.

19. The method of claim 17, wherein the associated gene is a proto-oncogene.

20. The method of claim 17, wherein the associated gene is located within an insulated neighborhood different from the insulated neighborhood in which the super-enhancer is located.

21. A method of identifying a super-enhancer in a 3D regulatory landscape of a cell comprising:
a. identifying genomic regions of DNA within the cell enriched for H3K27ac signal;
b. stitching the enriched regions together if within 12.5 kb of each other;
c. ranking stitched regions by H3K27ac signal; and
d. identifying a ranked stitched region as a super-enhancer if the ranked stitched region falls above a threshold at which two classes of enhancers are separable.

22. A method of identifying a disruption in an insulated neighborhood boundary comprising:
a. identifying a proto-oncogene of interest;
b. identifying an insulated neighborhood within which the proto-oncogene of interest is located; and
c. examining the proto-oncogene insulated neighborhood for disruptions in a proto-oncogene neighborhood boundary.

23. The method of claim 22, further comprising an enhancer, wherein the enhancer is located outside the proto-oncogene insulated neighborhood.

24. The method of claim 23, wherein the enhancer is a super-enhancer.

25. The method of claim 23, wherein the enhancer is located within an insulated neighborhood, and wherein the enhancer insulated neighborhood is different than the proto-oncogene insulated neighborhood.

26. The method of claim 22, further comprising identifying activation of a proto-oncogene located within the proto-oncogene insulated neighborhood by an enhancer located outside the proto-oncogene insulated neighborhood.

27. The method of claim 22, wherein the disruption in the proto-oncogene neighborhood boundary is a mutation in a CTCF-CTCF loop anchor region.

28. The method of claim 22, wherein the disruption in the proto-oncogene neighborhood boundary is a microdeletion in a CTCF-CTCF loop anchor region.

29. The method of claim 22, wherein the disruption is a deletion, and wherein the proto-oncogene neighborhood boundary overlaps the deletion by at least one 1 bp.

30. A method of identifying a disruption in an insulated neighborhood boundary comprising:
a. identifying at least one proto-oncogene of interest;
b. identifying candidate neighborhoods comprised of CTCF-CTCF loops wherein a transcription start site of the at least one proto-oncogene is located within the neighborhood;
c. examining the proto-oncogene neighborhoods for disruptions; and
d. determining if any identical disruptions overlap proto-oncogene neighborhood boundaries.

31. The method of claim 30, wherein the disruptions are deletions.

32. The method of claim 31, wherein the deletions are microdeletions.

33. The method of claim 30, wherein the disruptions are mutations.

34. A method of screening for cancer, comprising:
a. identifying a proto-oncogene of interest, wherein the proto-oncogene is located within an insulated neighborhood;
b. examining the proto-oncogene insulated neighborhood for disruptions in a boundary of the proto-oncogene insulated neighborhood; and
c. measuring expression of the proto-oncogene, wherein elevated levels of the proto-oncogene indicated a likelihood of cancer.

35. A method of treating a cancer involving an activated proto-oncogene, comprising administering to a patient in need of such treatment an effective amount of an agent that repairs a disruption in an insulated neighborhood boundary, wherein the activated proto-oncogene is located within an insulated neighborhood, thereby decreasing expression of the proto-oncogene such that the cancer is treated.

36. A method of treating a cancer involving an activated proto-oncogene, comprising administering to a patient in need of such treatment an effective amount of an agent that disrupts activation of a proto-oncogene, thereby decreasing expression of the proto-oncogene such that the cancer is treated.

37. The method of claim 36, further comprising, prior to administering an agent, identifying a cancer as comprising cells that have a disruption in an insulated neighborhood boundary.

38. The method of claim 36, wherein activation of the proto-oncogene occurs as a result of a disruption in an insulated neighborhood boundary.

39. A method of identifying an agent that stabilizes an insulated neighborhood, wherein the insulated neighborhood has a disrupted boundary, comprising:
a. transfecting a cell with a super-enhancer and the insulated neighborhood under conditions suitable for the super-enhancer to drive high levels of expression of a proto-oncogene associated with the super-enhancer and located within the insulated neighborhood;
b. contacting the cell with a test agent; and
c. measuring the level of expression of the proto-oncogene, wherein decreased expression of the proto-oncogene in the presence of the test agent indicates that the test agent is an agent that stabilizes an insulated neighborhood.

40. The method of claim 39, wherein the agent disrupts the super-enhancer associated with the proto-oncogene.

41. The method of claim 39, wherein the super-enhancer is located outside the insulated neighborhood.

42. The method of claim 39, wherein a nucleic acid comprises the super-enhancer and the insulated neighborhood and is transfected.

43. The method of claim 39, wherein the agent repairs a disruption in the disrupted insulated neighborhood boundary.

44. The method of claim 39, wherein expression of the proto-oncogene is measured at least in part by measuring the level of a gene product encoded by the proto-oncogene or by measuring activity of a gene product encoded by the proto-oncogene.

45. The method of claim 44, wherein a gene product is mRNA or polypeptide encoded by the gene.

46. A method of identifying an agent that disrupts a super-enhancer associated with a proto-oncogene, comprising:
a. transfecting a cell with a super-enhancer and an associated proto-oncogene under conditions suitable for the super-enhancer to drive high levels of expression of the proto-oncogene, wherein the proto-oncogene is located within an insulated neighborhood;
b. contacting the cell with a test agent; and
c. measuring the level of expression of the proto-oncogene, wherein decreased expression of the proto-oncogene in the presence of the test agent indicates that the test agent is an agent that disrupts the super-enhancer associated with the proto-oncogene.

47. A method of identifying a screening agent that identifies a disruption in an insulated neighborhood boundary, comprising:
a. transfecting a cell with a super-enhancer and an associated proto-oncogene, wherein the proto-oncogene is located within an insulated neighborhood;
b. contacting the cell with a screening agent; and
c. measuring the level of expression of the screening agent, wherein increased expression of the screening agent indicates that the proto-oncogene is activated.

48. A method of screening an individual for a pre-disposition of cancer comprising:
a. identifying a proto-oncogene located within an insulated neighborhood; and
b. determining if a boundary of the insulated neighborhood includes a disruption, wherein a disruption in the insulated neighborhood boundary indicates an increased risk of cancer.

49. The method of claim 48, further comprising identifying if an enhancer is located within the vicinity of the insulated neighborhood.

50. The method of claim 48, wherein the disruption in the insulated neighborhood boundary is a deletion in a CTCF loop binding site.

51. A method of identifying a candidate target for treating a cancer, comprising:
a. detecting a disrupted boundary of an insulated neighborhood that contains one or more proto-oncogenes in genomic DNA derived from the cancer; and
b. identifying an enhancer located outside of and in proximity to the insulated neighborhood, thereby identifying the proto-oncogene and the enhancer as candidate targets for treating the cancer.

52. The method of claim 51, wherein the enhancer is a super-enhancer.

53. The method of claim 51 or claim 52, wherein the proto-oncogene is not expressed when the insulated neighborhood boundary is not disrupted.

54. The method of any of claims 51-53, further comprising measuring expression of the proto-oncogene in a sample comprising cancer cells derived from the cancer, wherein higher expression of the proto-oncogene in cancer cells as compared to normal cells indicates that the proto-oncogene is a target for treating the cancer.

55. The method of any of claims 51-54, further comprising measuring expression of the proto-oncogene in a sample comprising cancer cells derived from the cancer, wherein higher expression of the proto-oncogene in cancer cells as compared to normal cells indicates that the enhancer is a target for treating the cancer.

56. The method of any of claims 51-54, further comprising identifying an agent that inhibits activity of the enhancer.

57. The method of any of claims 51-54, further comprising identifying an agent that inhibits expression of the proto-oncogene or inhibits activity of a gene product of the proto-oncogene.

58. The method of any of claims 51-57, further comprising contacting a cancer cell having a disruption in the insulated neighborhood boundary with an agent that inhibits activity of the enhancer, inhibits expression of the proto-oncogene, or inhibits activity of a gene product of the proto-oncogene.

59. The method of any of claims 45-51, further comprising administering to a subject in need of treatment for a cancer comprising cells that have a disruption in the insulated neighborhood boundary, an agent that inhibits activity of the enhancer, inhibits expression of the proto-oncogene, or inhibits activity of a gene product of the proto-oncogene.
